SEQUENCE LISTING (315 sequences)

SEQ ID NO: 1 - DNA, 20 bp, synthetic construct
  source 1..20: mol_type = other DNA
ggtatgtttcgtgcccatgt

SEQ ID NO: 2 - DNA, 20 bp, synthetic construct
  source 1..20: mol_type = other DNA
gtggtatgtttcgtgcccat

SEQ ID NO: 3 - DNA, 20 bp, synthetic construct
  source 1..20: mol_type = other DNA
tgtggtatgtttcgtgccca

SEQ ID NO: 4 - DNA, 20 bp, synthetic construct
  source 1..20: mol_type = other DNA
atgtggtatgtttcgtgccc

SEQ ID NO: 5 - DNA, 20 bp, synthetic construct
  source 1..20: mol_type = other DNA
aatgtggtatgtttcgtgcc

SEQ ID NO: 6 - DNA, 20 bp, synthetic construct
  source 1..20: mol_type = other DNA
aaatgtggtatgtttcgtgc

SEQ ID NO: 7 - DNA, 20 bp, synthetic construct
  source 1..20: mol_type = other DNA
aaaatgtggtatgtttcgtg

SEQ ID NO: 8 - DNA, 20 bp, synthetic construct
  source 1..20: mol_type = other DNA
taaaatgtggtatgtttcgt

SEQ ID NO: 9 - DNA, 20 bp, synthetic construct
  source 1..20: mol_type = other DNA
gtaaaatgtggtatgtttcg

SEQ ID NO: 10 - DNA, 20 bp, synthetic construct
  source 1..20: mol_type = other DNA
cgtaaaatgtggtatgtttc

SEQ ID NO: 11 - DNA, 20 bp, synthetic construct
  source 1..20: mol_type = other DNA
ccgtaaaatgtggtatgttt

SEQ ID NO: 12 - DNA, 20 bp, synthetic construct
  source 1..20: mol_type = other DNA
tccgtaaaatgtggtatgtt

SEQ ID NO: 13 - DNA, 20 bp, synthetic construct
  source 1..20: mol_type = other DNA
ggctacatcctccgtaaaat

SEQ ID NO: 14 - DNA, 20 bp, synthetic construct
  source 1..20: mol_type = other DNA
tcatttatgcttctccacct

SEQ ID NO: 15 - DNA, 20 bp, synthetic construct
  source 1..20: mol_type = other DNA
ttcatttatgcttctccacc

SEQ ID NO: 16 - DNA, 20 bp, synthetic construct
  source 1..20: mol_type = other DNA
tttcatttatgcttctccac

SEQ ID NO: 17 - DNA, 20 bp, synthetic construct
  source 1..20: mol_type = other DNA
ctttcatttatgcttctcca

SEQ ID NO: 18 - DNA, 20 bp, synthetic construct
  source 1..20: mol_type = other DNA
cctttcatttatgcttctcc

SEQ ID NO: 19 - DNA, 20 bp, synthetic construct
  source 1..20: mol_type = other DNA
gcctttcatttatgcttctc

SEQ ID NO: 20 - DNA, 20 bp, synthetic construct
  source 1..20: mol_type = other DNA
tgcctttcatttatgcttct

SEQ ID NO: 21 - DNA, 20 bp, synthetic construct
  source 1..20: mol_type = other DNA
gtgcctttcatttatgcttc

SEQ ID NO: 22 - DNA, 20 bp, synthetic construct
  source 1..20: mol_type = other DNA
ggtgcctttcatttatgctt

SEQ ID NO: 23 - DNA, 20 bp, synthetic construct
  source 1..20: mol_type = other DNA
aggtgcctttcatttatgct

SEQ ID NO: 24 - DNA, 20 bp, synthetic construct
  source 1..20: mol_type = other DNA
aaggtgcctttcatttatgc

SEQ ID NO: 25 - DNA, 20 bp, synthetic construct
  source 1..20: mol_type = other DNA
aattctctgtgcatcttcta

SEQ ID NO: 26 - DNA, 20 bp, synthetic construct
  source 1..20: mol_type = other DNA
aaattctctgtgcatcttct

SEQ ID NO: 27 - DNA, 20 bp, synthetic construct
  source 1..20: mol_type = other DNA
gaaattctctgtgcatcttc

SEQ ID NO: 28 - DNA, 20 bp, synthetic construct
  source 1..20: mol_type = other DNA
agaaattctctgtgcatctt

SEQ ID NO: 29 - DNA, 20 bp, synthetic construct
  source 1..20: mol_type = other DNA
cacaaggtcagaaattctct

SEQ ID NO: 30 - DNA, 20 bp, synthetic construct
  source 1..20: mol_type = other DNA
tcacaaggtcagaaattctc

SEQ ID NO: 31 - DNA, 20 bp, synthetic construct
  source 1..20: mol_type = other DNA
gtcacaaggtcagaaattct

SEQ ID NO: 32 - DNA, 20 bp, synthetic construct
  source 1..20: mol_type = other DNA
aacgtcacaaggtcagaaat

SEQ ID NO: 33 - DNA, 20 bp, synthetic construct
  source 1..20: mol_type = other DNA
tggatgcctctgtatgggat

SEQ ID NO: 34 - DNA, 20 bp, synthetic construct
  source 1..20: mol_type = other DNA
ctggatgcctctgtatggga

SEQ ID NO: 35 - DNA, 20 bp, synthetic construct
  source 1..20: mol_type = other DNA
acctggatgcctctgtatgg

SEQ ID NO: 36 - DNA, 20 bp, synthetic construct
  source 1..20: mol_type = other DNA
tacctggatgcctctgtatg

SEQ ID NO: 37 - DNA, 20 bp, synthetic construct
  source 1..20: mol_type = other DNA
atacctggatgcctctgtat

SEQ ID NO: 38 - DNA, 20 bp, synthetic construct
  source 1..20: mol_type = other DNA
aatacctggatgcctctgta

SEQ ID NO: 39 - DNA, 20 bp, synthetic construct
  source 1..20: mol_type = other DNA
aaatacctggatgcctctgt

SEQ ID NO: 40 - DNA, 20 bp, synthetic construct
  source 1..20: mol_type = other DNA
gaaatacctggatgcctctg

SEQ ID NO: 41 - DNA, 20 bp, synthetic construct
  source 1..20: mol_type = other DNA
ggaaatacctggatgcctct

SEQ ID NO: 42 - DNA, 20 bp, synthetic construct
  source 1..20: mol_type = other DNA
attatagacgagttggctcc

SEQ ID NO: 43 - DNA, 20 bp, synthetic construct
  source 1..20: mol_type = other DNA
gctgttaggaagatattata

SEQ ID NO: 44 - DNA, 20 bp, synthetic construct
  source 1..20: mol_type = other DNA
tgctgttaggaagatattat

SEQ ID NO: 45 - DNA, 20 bp, synthetic construct
  source 1..20: mol_type = other DNA
ctgctgttaggaagatatta

SEQ ID NO: 46 - DNA, 20 bp, synthetic construct
  source 1..20: mol_type = other DNA
tctgctgttaggaagatatt

SEQ ID NO: 47 - DNA, 20 bp, synthetic construct
  source 1..20: mol_type = other DNA
ttctgctgttaggaagatat

SEQ ID NO: 48 - DNA, 20 bp, synthetic construct
  source 1..20: mol_type = other DNA
gttctgctgttaggaagata

SEQ ID NO: 49 - DNA, 20 bp, synthetic construct
  source 1..20: mol_type = other DNA
ggttctgctgttaggaagat

SEQ ID NO: 50 - DNA, 20 bp, synthetic construct
  source 1..20: mol_type = other DNA
aggttctgctgttaggaaga

SEQ ID NO: 51 - DNA, 20 bp, synthetic construct
  source 1..20: mol_type = other DNA
caggttctgctgttaggaag

SEQ ID NO: 52 - DNA, 20 bp, synthetic construct
  source 1..20: mol_type = other DNA
ccaggttctgctgttaggaa

SEQ ID NO: 53 - DNA, 20 bp, synthetic construct
  source 1..20: mol_type = other DNA
cccaggttctgctgttagga

SEQ ID NO: 54 - DNA, 20 bp, synthetic construct
  source 1..20: mol_type = other DNA
acccaggttctgctgttagg

SEQ ID NO: 55 - DNA, 20 bp, synthetic construct
  source 1..20: mol_type = other DNA
gagacccaggttctgctgtt

SEQ ID NO: 56 - DNA, 20 bp, synthetic construct
  source 1..20: mol_type = other DNA
tgagacccaggttctgctgt

SEQ ID NO: 57 - DNA, 20 bp, synthetic construct
  source 1..20: mol_type = other DNA
ccgtacctgtagttccagct

SEQ ID NO: 58 - DNA, 20 bp, synthetic construct
  source 1..20: mol_type = other DNA
tcccgtacctgtagttccag

SEQ ID NO: 59 - DNA, 20 bp, synthetic construct
  source 1..20: mol_type = other DNA
ttcccgtacctgtagttcca

SEQ ID NO: 60 - DNA, 20 bp, synthetic construct
  source 1..20: mol_type = other DNA
tttcccgtacctgtagttcc

SEQ ID NO: 61 - DNA, 20 bp, synthetic construct
  source 1..20: mol_type = other DNA
ttttcccgtacctgtagttc

SEQ ID NO: 62 - DNA, 20 bp, synthetic construct
  source 1..20: mol_type = other DNA
gttttcccgtacctgtagtt

SEQ ID NO: 63 - DNA, 20 bp, synthetic construct
  source 1..20: mol_type = other DNA
agttttcccgtacctgtagt

SEQ ID NO: 64 - DNA, 20 bp, synthetic construct
  source 1..20: mol_type = other DNA
ccgaaattattttgttaaac

SEQ ID NO: 65 - DNA, 20 bp, synthetic construct
  source 1..20: mol_type = other DNA
gccgaaattattttgttaaa

SEQ ID NO: 66 - DNA, 20 bp, synthetic construct
  source 1..20: mol_type = other DNA
agccgaaattattttgttaa

SEQ ID NO: 67 - DNA, 20 bp, synthetic construct
  source 1..20: mol_type = other DNA
tagccgaaattattttgtta

SEQ ID NO: 68 - DNA, 20 bp, synthetic construct
  source 1..20: mol_type = other DNA
atagccgaaattattttgtt

SEQ ID NO: 69 - DNA, 20 bp, synthetic construct
  source 1..20: mol_type = other DNA
gatagccgaaattattttgt

SEQ ID NO: 70 - DNA, 20 bp, synthetic construct
  source 1..20: mol_type = other DNA
ttgatagccgaaattatttt

SEQ ID NO: 71 - DNA, 20 bp, synthetic construct
  source 1..20: mol_type = other DNA
tttgatagccgaaattattt

SEQ ID NO: 72 - DNA, 20 bp, synthetic construct
  source 1..20: mol_type = other DNA
ctttgatagccgaaattatt

SEQ ID NO: 73 - DNA, 20 bp, synthetic construct
  source 1..20: mol_type = other DNA
tctttgatagccgaaattat

SEQ ID NO: 74 - DNA, 20 bp, synthetic construct
  source 1..20: mol_type = other DNA
gatctttgatagccgaaatt

SEQ ID NO: 75 - DNA, 20 bp, synthetic construct
  source 1..20: mol_type = other DNA
tgatctttgatagccgaaat

SEQ ID NO: 76 - DNA, 20 bp, synthetic construct
  source 1..20: mol_type = other DNA
ttgatctttgatagccgaaa

SEQ ID NO: 77 - DNA, 20 bp, synthetic construct
  source 1..20: mol_type = other DNA
cttgatctttgatagccgaa

SEQ ID NO: 78 - DNA, 20 bp, synthetic construct
  source 1..20: mol_type = other DNA
acttgatctttgatagccga

SEQ ID NO: 79 - DNA, 20 bp, synthetic construct
  source 1..20: mol_type = other DNA
cacttgatctttgatagccg

SEQ ID NO: 80 - DNA, 20 bp, synthetic construct
  source 1..20: mol_type = other DNA
ccacttgatctttgatagcc

SEQ ID NO: 81 - DNA, 20 bp, synthetic construct
  source 1..20: mol_type = other DNA
tatcccacttgatctttgat

SEQ ID NO: 82 - DNA, 20 bp, synthetic construct
  source 1..20: mol_type = other DNA
ttatcccacttgatctttga

SEQ ID NO: 83 - DNA, 20 bp, synthetic construct
  source 1..20: mol_type = other DNA
atttatcccacttgatcttt

SEQ ID NO: 84 - DNA, 20 bp, synthetic construct
  source 1..20: mol_type = other DNA
aatttatcccacttgatctt

SEQ ID NO: 85 - DNA, 20 bp, synthetic construct
  source 1..20: mol_type = other DNA
cctctatggtatgcaaggag

SEQ ID NO: 86 - DNA, 20 bp, synthetic construct
  source 1..20: mol_type = other DNA
acctcgacctctcctctatg

SEQ ID NO: 87 - DNA, 20 bp, synthetic construct
  source 1..20: mol_type = other DNA
gacctcgacctctcctctat

SEQ ID NO: 88 - DNA, 20 bp, synthetic construct
  source 1..20: mol_type = other DNA
gctagcagactcacaccaca

SEQ ID NO: 89 - DNA, 20 bp, synthetic construct
  source 1..20: mol_type = other DNA
tcacggctagcagactcaca

SEQ ID NO: 90 - DNA, 20 bp, synthetic construct
  source 1..20: mol_type = other DNA
tgtctctcacggctagcaga

SEQ ID NO: 91 - DNA, 20 bp, synthetic construct
  source 1..20: mol_type = other DNA
tctgtctctcacggctagca

SEQ ID NO: 92 - DNA, 20 bp, synthetic construct
  source 1..20: mol_type = other DNA
atctgtctctcacggctagc

SEQ ID NO: 93 - DNA, 20 bp, synthetic construct
  source 1..20: mol_type = other DNA
ccctttgtaatgcatctgtc

SEQ ID NO: 94 - DNA, 20 bp, synthetic construct
  source 1..20: mol_type = other DNA
tccctttgtaatgcatctgt

SEQ ID NO: 95 - DNA, 20 bp, synthetic construct
  source 1..20: mol_type = other DNA
atccctttgtaatgcatctg

SEQ ID NO: 96 - DNA, 20 bp, synthetic construct
  source 1..20: mol_type = other DNA
catccctttgtaatgcatct

SEQ ID NO: 97 - DNA, 20 bp, synthetic construct
  source 1..20: mol_type = other DNA
ccatccctttgtaatgcatc

SEQ ID NO: 98 - DNA, 20 bp, synthetic construct
  source 1..20: mol_type = other DNA
tccatccctttgtaatgcat

SEQ ID NO: 99 - DNA, 20 bp, synthetic construct
  source 1..20: mol_type = other DNA
atccatccctttgtaatgca

SEQ ID NO: 100 - DNA, 20 bp, synthetic construct
  source 1..20: mol_type = other DNA
aatccatccctttgtaatgc

SEQ ID NO: 101 - DNA, 20 bp, synthetic construct
  source 1..20: mol_type = other DNA
aaatccatccctttgtaatg

SEQ ID NO: 102 - DNA, 20 bp, synthetic construct
  source 1..20: mol_type = other DNA
taaatccatccctttgtaat

SEQ ID NO: 103 - DNA, 20 bp, synthetic construct
  source 1..20: mol_type = other DNA
ctaaatccatccctttgtaa

SEQ ID NO: 104 - DNA, 20 bp, synthetic construct
  source 1..20: mol_type = other DNA
actaaatccatccctttgta

SEQ ID NO: 105 - DNA, 20 bp, synthetic construct
  source 1..20: mol_type = other DNA
cactaaatccatccctttgt

SEQ ID NO: 106 - DNA, 20 bp, synthetic construct
  source 1..20: mol_type = other DNA
gcactaaatccatccctttg

SEQ ID NO: 107 - DNA, 20 bp, synthetic construct
  source 1..20: mol_type = other DNA
tgcactaaatccatcccttt

SEQ ID NO: 108 - DNA, 20 bp, synthetic construct
  source 1..20: mol_type = other DNA
gtgcactaaatccatccctt

SEQ ID NO: 109 - DNA, 20 bp, synthetic construct
  source 1..20: mol_type = other DNA
agtgcactaaatccatccct

SEQ ID NO: 110 - DNA, 20 bp, synthetic construct
  source 1..20: mol_type = other DNA
gttttgtttcattgttcact

SEQ ID NO: 111 - DNA, 20 bp, synthetic construct
  source 1..20: mol_type = other DNA
agttttgtttcattgttcac

SEQ ID NO: 112 - DNA, 20 bp, synthetic construct
  source 1..20: mol_type = other DNA
aagttttgtttcattgttca

SEQ ID NO: 113 - DNA, 20 bp, synthetic construct
  source 1..20: mol_type = other DNA
cttgggaagaagttttgttt

SEQ ID NO: 114 - DNA, 20 bp, synthetic construct
  source 1..20: mol_type = other DNA
gcttgggaagaagttttgtt

SEQ ID NO: 115 - DNA, 20 bp, synthetic construct
  source 1..20: mol_type = other DNA
atctcttcaaactatggcac

SEQ ID NO: 116 - DNA, 20 bp, synthetic construct
  source 1..20: mol_type = other DNA
catctcttcaaactatggca

SEQ ID NO: 117 - DNA, 20 bp, synthetic construct
  source 1..20: mol_type = other DNA
tgccatctcttcaaactatg

SEQ ID NO: 118 - DNA, 20 bp, synthetic construct
  source 1..20: mol_type = other DNA
atgccatctcttcaaactat

SEQ ID NO: 119 - DNA, 20 bp, synthetic construct
  source 1..20: mol_type = other DNA
gatgccatctcttcaaacta

SEQ ID NO: 120 - DNA, 20 bp, synthetic construct
  source 1..20: mol_type = other DNA
ttgtataaactgttgttgca

SEQ ID NO: 121 - DNA, 20 bp, synthetic construct
  source 1..20: mol_type = other DNA
gaagctgaagtggtgttggg

SEQ ID NO: 122 - DNA, 20 bp, synthetic construct
  source 1..20: mol_type = other DNA
agaagctgaagtggtgttgg

SEQ ID NO: 123 - DNA, 20 bp, synthetic construct
  source 1..20: mol_type = other DNA
cttttcctcggcatccttcg

SEQ ID NO: 124 - DNA, 20 bp, synthetic construct
  source 1..20: mol_type = other DNA
ccttttcctcggcatccttc

SEQ ID NO: 125 - DNA, 20 bp, synthetic construct
  source 1..20: mol_type = other DNA
tccttttcctcggcatcctt

SEQ ID NO: 126 - DNA, 20 bp, synthetic construct
  source 1..20: mol_type = other DNA
atccttttcctcggcatcct

SEQ ID NO: 127 - DNA, 20 bp, synthetic construct
  source 1..20: mol_type = other DNA
tatccttttcctcggcatcc

SEQ ID NO: 128 - DNA, 20 bp, synthetic construct
  source 1..20: mol_type = other DNA
atatccttttcctcggcatc

SEQ ID NO: 129 - DNA, 20 bp, synthetic construct
  source 1..20: mol_type = other DNA
gatatccttttcctcggcat

SEQ ID NO: 130 - DNA, 20 bp, synthetic construct
  source 1..20: mol_type = other DNA
tgatatccttttcctcggca

SEQ ID NO: 131 - DNA, 20 bp, synthetic construct
  source 1..20: mol_type = other DNA
ccgatgctctggaatctcaa

SEQ ID NO: 132 - DNA, 20 bp, synthetic construct
  source 1..20: mol_type = other DNA
tccgatgctctggaatctca

SEQ ID NO: 133 - DNA, 20 bp, synthetic construct
  source 1..20: mol_type = other DNA
catccgatgctctggaatct

SEQ ID NO: 134 - DNA, 20 bp, synthetic construct
  source 1..20: mol_type = other DNA
tcatccgatgctctggaatc

SEQ ID NO: 135 - DNA, 20 bp, synthetic construct
  source 1..20: mol_type = other DNA
ttcatccgatgctctggaat

SEQ ID NO: 136 - DNA, 20 bp, synthetic construct
  source 1..20: mol_type = other DNA
actacccctatgcacgtgag

SEQ ID NO: 137 - DNA, 20 bp, synthetic construct
  source 1..20: mol_type = other DNA
gttcttccccaaatgccttt

SEQ ID NO: 138 - DNA, 20 bp, synthetic construct
  source 1..20: mol_type = other DNA
tgttcttccccaaatgcctt

SEQ ID NO: 139 - DNA, 20 bp, synthetic construct
  source 1..20: mol_type = other DNA
ttgttcttccccaaatgcct

SEQ ID NO: 140 - DNA, 20 bp, synthetic construct
  source 1..20: mol_type = other DNA
gttgttcttccccaaatgcc

SEQ ID NO: 141 - DNA, 20 bp, synthetic construct
  source 1..20: mol_type = other DNA
cgttgttcttccccaaatgc

SEQ ID NO: 142 - DNA, 20 bp, synthetic construct
  source 1..20: mol_type = other DNA
ctttctctggagacacatca

SEQ ID NO: 143 - DNA, 20 bp, synthetic construct
  source 1..20: mol_type = other DNA
actttctctggagacacatc

SEQ ID NO: 144 - DNA, 20 bp, synthetic construct
  source 1..20: mol_type = other DNA
gttgtttgtttgtttgtttt

SEQ ID NO: 145 - DNA, 20 bp, synthetic construct
  source 1..20: mol_type = other DNA
tgttgtttgtttgtttgttt

SEQ ID NO: 146 - DNA, 20 bp, synthetic construct
  source 1..20: mol_type = other DNA
ttgttgtttgtttgtttgtt

SEQ ID NO: 147 - DNA, 20 bp, synthetic construct
  source 1..20: mol_type = other DNA
gttgttgtttgtttgtttgt

SEQ ID NO: 148 - DNA, 20 bp, synthetic construct
  source 1..20: mol_type = other DNA
tgttgttgtttgtttgtttg

SEQ ID NO: 149 - DNA, 20 bp, synthetic construct
  source 1..20: mol_type = other DNA
ttgttgttgtttgtttgttt

SEQ ID NO: 150 - DNA, 20 bp, synthetic construct
  source 1..20: mol_type = other DNA
attgttgttgtttgtttgtt

SEQ ID NO: 151 - DNA, 20 bp, synthetic construct
  source 1..20: mol_type = other DNA
tattgttgttgtttgtttgt

SEQ ID NO: 152 - DNA, 20 bp, synthetic construct
  source 1..20: mol_type = other DNA
ttattgttgttgtttgtttg

SEQ ID NO: 153 - DNA, 20 bp, synthetic construct
  source 1..20: mol_type = other DNA
gtttattgttgttgtttgtt

SEQ ID NO: 154 - DNA, 20 bp, synthetic construct
  source 1..20: mol_type = other DNA
tgtttattgttgttgtttgt

SEQ ID NO: 155 - DNA, 20 bp, synthetic construct
  source 1..20: mol_type = other DNA
ttgtttattgttgttgtttg

SEQ ID NO: 156 - DNA, 20 bp, synthetic construct
  source 1..20: mol_type = other DNA
gttgtttattgttgttgttt

SEQ ID NO: 157 - DNA, 20 bp, synthetic construct
  source 1..20: mol_type = other DNA
agttgtttattgttgttgtt

SEQ ID NO: 158 - DNA, 20 bp, synthetic construct
  source 1..20: mol_type = other DNA
aagttgtttattgttgttgt

SEQ ID NO: 159 - DNA, 20 bp, synthetic construct
  source 1..20: mol_type = other DNA
agtggaatgagtcagcccga

SEQ ID NO: 160 - DNA, 20 bp, synthetic construct
  source 1..20: mol_type = other DNA
aagtggaatgagtcagcccg

SEQ ID NO: 161 - DNA, 20 bp, synthetic construct
  source 1..20: mol_type = other DNA
aaagtggaatgagtcagccc

SEQ ID NO: 162 - DNA, 20 bp, synthetic construct
  source 1..20: mol_type = other DNA
cctgctggataggaattaat

SEQ ID NO: 163 - DNA, 20 bp, synthetic construct
  source 1..20: mol_type = other DNA
gcctgctggataggaattaa

SEQ ID NO: 164 - DNA, 20 bp, synthetic construct
  source 1..20: mol_type = other DNA
ttaaagcctgctggatagga

SEQ ID NO: 165 - DNA, 20 bp, synthetic construct
  source 1..20: mol_type = other DNA
tgttaaagcctgctggatag

SEQ ID NO: 166 - DNA, 20 bp, synthetic construct
  source 1..20: mol_type = other DNA
ttgttaaagcctgctggata

SEQ ID NO: 167 - DNA, 20 bp, synthetic construct
  source 1..20: mol_type = other DNA
tttgttaaagcctgctggat

SEQ ID NO: 168 - DNA, 20 bp, synthetic construct
  source 1..20: mol_type = other DNA
ttttgttaaagcctgctgga

SEQ ID NO: 169 - DNA, 20 bp, synthetic construct
  source 1..20: mol_type = other DNA
tttttgttaaagcctgctgg

SEQ ID NO: 170 - DNA, 20 bp, synthetic construct
  source 1..20: mol_type = other DNA
gtttttgttaaagcctgctg

SEQ ID NO: 171 - DNA, 20 bp, synthetic construct
  source 1..20: mol_type = other DNA
agtttttgttaaagcctgct

SEQ ID NO: 172 - DNA, 20 bp, synthetic construct
  source 1..20: mol_type = other DNA
tagtttttgttaaagcctgc

SEQ ID NO: 173 - DNA, 20 bp, synthetic construct
  source 1..20: mol_type = other DNA
tctttagtagctttcatggc

SEQ ID NO: 174 - DNA, 20 bp, synthetic construct
  source 1..20: mol_type = other DNA
ctggcttttctttagtagct

SEQ ID NO: 175 - DNA, 20 bp, synthetic construct
  source 1..20: mol_type = other DNA
gctgtttctggcttttcttt

SEQ ID NO: 176 - DNA, 20 bp, synthetic construct
  source 1..20: mol_type = other DNA
cgctgtttctggcttttctt

SEQ ID NO: 177 - DNA, 20 bp, synthetic construct
  source 1..20: mol_type = other DNA
acgctgtttctggcttttct

SEQ ID NO: 178 - DNA, 20 bp, synthetic construct
  source 1..20: mol_type = other DNA
tacgctgtttctggcttttc

SEQ ID NO: 179 - DNA, 20 bp, synthetic construct
  source 1..20: mol_type = other DNA
ttacgctgtttctggctttt

SEQ ID NO: 180 - DNA, 20 bp, synthetic construct
  source 1..20: mol_type = other DNA
cttacgctgtttctggcttt

SEQ ID NO: 181 - DNA, 20 bp, synthetic construct
  source 1..20: mol_type = other DNA
tcttacgctgtttctggctt

SEQ ID NO: 182 - DNA, 20 bp, synthetic construct
  source 1..20: mol_type = other DNA
ttcttacgctgtttctggct

SEQ ID NO: 183 - DNA, 20 bp, synthetic construct
  source 1..20: mol_type = other DNA
attcttacgctgtttctggc

SEQ ID NO: 184 - DNA, 20 bp, synthetic construct
  source 1..20: mol_type = other DNA
cctcgtctctgaatcatatt

SEQ ID NO: 185 - DNA, 20 bp, synthetic construct
  source 1..20: mol_type = other DNA
cacaatagtagtggccttgt

SEQ ID NO: 186 - DNA, 20 bp, synthetic construct
  source 1..20: mol_type = other DNA
tcacaatagtagtggccttg

SEQ ID NO: 187 - DNA, 20 bp, synthetic construct
  source 1..20: mol_type = other DNA
ttcacaatagtagtggcctt

SEQ ID NO: 188 - DNA, 20 bp, synthetic construct
  source 1..20: mol_type = other DNA
attcacaatagtagtggcct

SEQ ID NO: 189 - DNA, 20 bp, synthetic construct
  source 1..20: mol_type = other DNA
ccatgttgacttagttggtc

SEQ ID NO: 190 - DNA, 20 bp, synthetic construct
  source 1..20: mol_type = other DNA
accatgttgacttagttggt

SEQ ID NO: 191 - DNA, 20 bp, synthetic construct
  source 1..20: mol_type = other DNA
gctaccatgtctgactaatt

SEQ ID NO: 192 - DNA, 20 bp, synthetic construct
  source 1..20: mol_type = other DNA
tgctaccatgtctgactaat

SEQ ID NO: 193 - DNA, 20 bp, synthetic construct
  source 1..20: mol_type = other DNA
gtgctaccatgtctgactaa

SEQ ID NO: 194 - DNA, 20 bp, synthetic construct
  source 1..20: mol_type = other DNA
tgtgctaccatgtctgacta

SEQ ID NO: 195 - DNA, 20 bp, synthetic construct
  source 1..20: mol_type = other DNA
atgtgctaccatgtctgact

SEQ ID NO: 196 - DNA, 20 bp, synthetic construct
  source 1..20: mol_type = other DNA
catgtgctaccatgtctgac

SEQ ID NO: 197 - DNA, 20 bp, synthetic construct
  source 1..20: mol_type = other DNA
tgggtgatatttggttccaa

SEQ ID NO: 198 - DNA, 20 bp, synthetic construct
  source 1..20: mol_type = other DNA
ctgaggaaattgatggtata

SEQ ID NO: 199 - DNA, 20 bp, synthetic construct
  source 1..20: mol_type = other DNA
actgaggaaattgatggtat

SEQ ID NO: 200 - DNA, 20 bp, synthetic construct
  source 1..20: mol_type = other DNA
gaccgtacgagggaatttta

SEQ ID NO: 201 - DNA, 20 bp, synthetic construct
  source 1..20: mol_type = other DNA
tgaccgtacgagggaatttt

SEQ ID NO: 202 - DNA, 20 bp, synthetic construct
  source 1..20: mol_type = other DNA
ttgaccgtacgagggaattt

SEQ ID NO: 203 - DNA, 20 bp, synthetic construct
  source 1..20: mol_type = other DNA
tttgaccgtacgagggaatt

SEQ ID NO: 204 - DNA, 20 bp, synthetic construct
  source 1..20: mol_type = other DNA
ttttgaccgtacgagggaat

SEQ ID NO: 205 - DNA, 20 bp, synthetic construct
  source 1..20: mol_type = other DNA
gccttctgtactgtgatggg

SEQ ID NO: 206 - DNA, 20 bp, synthetic construct
  source 1..20: mol_type = other DNA
agccttctgtactgtgatgg

SEQ ID NO: 207 - DNA, 20 bp, synthetic construct
  source 1..20: mol_type = other DNA
aagccttctgtactgtgatg

SEQ ID NO: 208 - DNA, 20 bp, synthetic construct
  source 1..20: mol_type = other DNA
atgtgtgggatgtaggtagg

SEQ ID NO: 209 - DNA, 20 bp, synthetic construct
  source 1..20: mol_type = other DNA
aatgtgtgggatgtaggtag

SEQ ID NO: 210 - DNA, 20 bp, synthetic construct
  source 1..20: mol_type = other DNA
aaatgtgtgggatgtaggta

SEQ ID NO: 211 - DNA, 20 bp, synthetic construct
  source 1..20: mol_type = other DNA
gggactcctgaaatgtgtgg

SEQ ID NO: 212 - DNA, 20 bp, synthetic construct
  source 1..20: mol_type = other DNA
cgttctgtgttttgtagaat

SEQ ID NO: 213 - DNA, 20 bp, synthetic construct
  source 1..20: mol_type = other DNA
gtcgttctgtgttttgtaga

SEQ ID NO: 214 - DNA, 20 bp, synthetic construct
  source 1..20: mol_type = other DNA
ggtcgttctgtgttttgtag

SEQ ID NO: 215 - DNA, 20 bp, synthetic construct
  source 1..20: mol_type = other DNA
tggtcgttctgtgttttgta

SEQ ID NO: 216 - DNA, 20 bp, synthetic construct
  source 1..20: mol_type = other DNA
atggtcgttctgtgttttgt

SEQ ID NO: 217 - DNA, 20 bp, synthetic construct
  source 1..20: mol_type = other DNA
tatggtcgttctgtgttttg

SEQ ID NO: 218 - DNA, 20 bp, synthetic construct
  source 1..20: mol_type = other DNA
atatggtcgttctgtgtttt

SEQ ID NO: 219 - DNA, 20 bp, synthetic construct
  source 1..20: mol_type = other DNA
tggctcatatggtcgttctg

SEQ ID NO: 220 - DNA, 20 bp, synthetic construct
  source 1..20: mol_type = other DNA
gtggctcatatggtcgttct

SEQ ID NO: 221 - DNA, 20 bp, synthetic construct
  source 1..20: mol_type = other DNA
agtggctcatatggtcgttc

SEQ ID NO: 222 - DNA, 20 bp, synthetic construct
  source 1..20: mol_type = other DNA
aagtggctcatatggtcgtt

SEQ ID NO: 223 - DNA, 20 bp, synthetic construct
  source 1..20: mol_type = other DNA
ctcagtgacagctaggtgga

SEQ ID NO: 224 - DNA, 20 bp, synthetic construct
  source 1..20: mol_type = other DNA
tctcagtgacagctaggtgg

SEQ ID NO: 225 - DNA, 20 bp, synthetic construct
  source 1..20: mol_type = other DNA
attctcagtgacagctaggt

SEQ ID NO: 226 - DNA, 20 bp, synthetic construct
  source 1..20: mol_type = other DNA
ccgaattctcagtgacagct

SEQ ID NO: 227 - DNA, 20 bp, synthetic construct
  source 1..20: mol_type = other DNA
caatgcagagtttctattac

SEQ ID NO: 228 - DNA, 20 bp, synthetic construct
  source 1..20: mol_type = other DNA
cccattcccaggatgttaga

SEQ ID NO: 229 - DNA, 20 bp, synthetic construct
  source 1..20: mol_type = other DNA
tcccattcccaggatgttag

SEQ ID NO: 230 - DNA, 20 bp, synthetic construct
  source 1..20: mol_type = other DNA
ttcccattcccaggatgtta

SEQ ID NO: 231 - DNA, 20 bp, synthetic construct
  source 1..20: mol_type = other DNA
cttcccattcccaggatgtt

SEQ ID NO: 232 - DNA, 20 bp, synthetic construct
  source 1..20: mol_type = other DNA
acttcccattcccaggatgt

SEQ ID NO: 233 - DNA, 20 bp, synthetic construct
  source 1..20: mol_type = other DNA
tacttcccattcccaggatg

SEQ ID NO: 234 - DNA, 20 bp, synthetic construct
  source 1..20: mol_type = other DNA
ttacttcccattcccaggat

SEQ ID NO: 235 - DNA, 20 bp, synthetic construct
  source 1..20: mol_type = other DNA
gttacttcccattcccagga

SEQ ID NO: 236 - DNA, 20 bp, synthetic construct
  source 1..20: mol_type = other DNA
tgttacttcccattcccagg

SEQ ID NO: 237 - DNA, 20 bp, synthetic construct
  source 1..20: mol_type = other DNA
gtgttacttcccattcccag

SEQ ID NO: 238 - DNA, 20 bp, synthetic construct
  source 1..20: mol_type = other DNA
agtgttacttcccattccca

SEQ ID NO: 239 - DNA, 20 bp, synthetic construct
  source 1..20: mol_type = other DNA
cagtgttacttcccattccc

SEQ ID NO: 240 - DNA, 20 bp, synthetic construct
  source 1..20: mol_type = other DNA
ccaccgatcccagtgttact

SEQ ID NO: 241 - DNA, 20 bp, synthetic construct
  source 1..20: mol_type = other DNA
tttccattcctctcttccat

SEQ ID NO: 242 - DNA, 20 bp, synthetic construct
  source 1..20: mol_type = other DNA
ctttccattcctctcttcca

SEQ ID NO: 243 - DNA, 20 bp, synthetic construct
  source 1..20: mol_type = other DNA
gcctttccattcctctcttc

SEQ ID NO: 244 - DNA, 20 bp, synthetic construct
  source 1..20: mol_type = other DNA
tgcctttccattcctctctt

SEQ ID NO: 245 - DNA, 20 bp, synthetic construct
  source 1..20: mol_type = other DNA
ttgcctttccattcctctct

SEQ ID NO: 246 - DNA, 20 bp, synthetic construct
  source 1..20: mol_type = other DNA
tttgcctttccattcctctc

SEQ ID NO: 247 - DNA, 20 bp, synthetic construct
  source 1..20: mol_type = other DNA
ttttgcctttccattcctct

SEQ ID NO: 248 - DNA, 20 bp, synthetic construct
  source 1..20: mol_type = other DNA
cttttgcctttccattcctc

SEQ ID NO: 249 - DNA, 20 bp, synthetic construct
  source 1..20: mol_type = other DNA
tcttttgcctttccattcct

SEQ ID NO: 250 - DNA, 20 bp, synthetic construct
  source 1..20: mol_type = other DNA
ttcttttgcctttccattcc

SEQ ID NO: 251 - DNA, 20 bp, synthetic construct
  source 1..20: mol_type = other DNA
tgctgatggtgggacttttt

SEQ ID NO: 252 - DNA, 20 bp, synthetic construct
  source 1..20: mol_type = other DNA
ttgctgatggtgggactttt

SEQ ID NO: 253 - DNA, 20 bp, synthetic construct
  source 1..20: mol_type = other DNA
tttgctgatggtgggacttt

SEQ ID NO: 254 - DNA, 20 bp, synthetic construct
  source 1..20: mol_type = other DNA
ttttgctgatggtgggactt

SEQ ID NO: 255 - DNA, 20 bp, synthetic construct
  source 1..20: mol_type = other DNA
cttttgctgatggtgggact

SEQ ID NO: 256 - DNA, 20 bp, synthetic construct
  source 1..20: mol_type = other DNA
tcttttgctgatggtgggac

SEQ ID NO: 257 - DNA, 20 bp, synthetic construct
  source 1..20: mol_type = other DNA
ttcttttgctgatggtggga

SEQ ID NO: 258 - DNA, 20 bp, synthetic construct
  source 1..20: mol_type = other DNA
cttcttttgctgatggtggg

SEQ ID NO: 259 - DNA, 20 bp, synthetic construct
  source 1..20: mol_type = other DNA
acttcttttgctgatggtgg

SEQ ID NO: 260 - DNA, 20 bp, synthetic construct
  source 1..20: mol_type = other DNA
gacttcttttgctgatggtg

SEQ ID NO: 261 - DNA, 20 bp, synthetic construct
  source 1..20: mol_type = other DNA
agacttcttttgctgatggt

SEQ ID NO: 262 - DNA, 20 bp, synthetic construct
  source 1..20: mol_type = other DNA
gagacttcttttgctgatgg

SEQ ID NO: 263 - DNA, 20 bp, synthetic construct
  source 1..20: mol_type = other DNA
agagacttcttttgctgatg

SEQ ID NO: 264 - DNA, 20 bp, synthetic construct
  source 1..20: mol_type = other DNA
gctgctattttagaggaaga

SEQ ID NO: 265 - DNA, 20 bp, synthetic construct
  source 1..20: mol_type = other DNA
ggctgctattttagaggaag

SEQ ID NO: 266 - DNA, 20 bp, synthetic construct
  source 1..20: mol_type = other DNA
ctttggctgctattttagag

SEQ ID NO: 267 - DNA, 20 bp, synthetic construct
  source 1..20: mol_type = other DNA
ctctttggctgctattttag

SEQ ID NO: 268 - DNA, 20 bp, synthetic construct
  source 1..20: mol_type = other DNA
tctctttggctgctatttta

SEQ ID NO: 269 - DNA, 20 bp, synthetic construct
  source 1..20: mol_type = other DNA
attttctctctttggctgct

SEQ ID NO: 270 - DNA, 20 bp, synthetic construct
  source 1..20: mol_type = other DNA
gttcagaaattgggattaat

SEQ ID NO: 271 - DNA, 20 bp, synthetic construct
  source 1..20: mol_type = other DNA
tgttcagaaattgggattaa

SEQ ID NO: 272 - DNA, 20 bp, synthetic construct
  source 1..20: mol_type = other DNA
gctgttcagaaattgggatt

SEQ ID NO: 273 - DNA, 20 bp, synthetic construct
  source 1..20: mol_type = other DNA
tgctgttcagaaattgggat

SEQ ID NO: 274 - DNA, 20 bp, synthetic construct
  source 1..20: mol_type = other DNA
atgctgttcagaaattggga

SEQ ID NO: 275 - DNA, 20 bp, synthetic construct
  source 1..20: mol_type = other DNA
aatgctgttcagaaattggg

SEQ ID NO: 276 - DNA, 20 bp, synthetic construct
  source 1..20: mol_type = other DNA
gctaagtaaaatgctgttca

SEQ ID NO: 277 - DNA, 20 bp, synthetic construct
  source 1..20: mol_type = other DNA
tgctaagtaaaatgctgttc

SEQ ID NO: 278 - DNA, 20 bp, synthetic construct
  source 1..20: mol_type = other DNA
tttccaacaggctctcgttt

SEQ ID NO: 279 - DNA, 20 bp, synthetic construct
  source 1..20: mol_type = other DNA
ctttccaacaggctctcgtt

SEQ ID NO: 280 - DNA, 20 bp, synthetic construct
  source 1..20: mol_type = other DNA
cctttccaacaggctctcgt

SEQ ID NO: 281 - DNA, 20 bp, synthetic construct
  source 1..20: mol_type = other DNA
tcctttccaacaggctctcg

SEQ ID NO: 282 - DNA, 20 bp, synthetic construct
  source 1..20: mol_type = other DNA
ggtagaatgggaaaggtttt

SEQ ID NO: 283 - DNA, 20 bp, synthetic construct
  source 1..20: mol_type = other DNA
gggtagaatgggaaaggttt

SEQ ID NO: 284 - DNA, 20 bp, synthetic construct
  source 1..20: mol_type = other DNA
tgggtagaatgggaaaggtt

SEQ ID NO: 285 - DNA, 20 bp, synthetic construct
  source 1..20: mol_type = other DNA
ctgggtagaatgggaaaggt

SEQ ID NO: 286 - DNA, 20 bp, synthetic construct
  source 1..20: mol_type = other DNA
gcacaagtggcaaagcaaaa

SEQ ID NO: 287 - DNA, 20 bp, synthetic construct
  source 1..20: mol_type = other DNA
tgcacaagtggcaaagcaaa

SEQ ID NO: 288 - DNA, 20 bp, synthetic construct
  source 1..20: mol_type = other DNA
agatctgttgcacaagtggc

SEQ ID NO: 289 - DNA, 15 bp, synthetic construct
  source 1..15: mol_type = other DNA
ggccagcttggcccg

SEQ ID NO: 290 - DNA, 16 bp, synthetic construct
  source 1..16: mol_type = other DNA
gcgcaccgcgcttgaa

SEQ ID NO: 291 - DNA, 17 bp, synthetic construct
  source 1..17: mol_type = other DNA
gccggtggaggtgaggc

SEQ ID NO: 292 - DNA, 19 bp, synthetic construct
  source 1..19: mol_type = other DNA
cgcggtccatgaaggtgag

SEQ ID NO: 293 - DNA, 18 bp, synthetic construct
  source 1..18: mol_type = other DNA
gccagtagagggagccgg

SEQ ID NO: 294 - DNA, 19 bp, synthetic construct
  source 1..19: mol_type = other DNA
gacaggaagggcgacatgg

SEQ ID NO: 295 - DNA, 16 bp, synthetic construct
  source 1..16: mol_type = other DNA
gcgggggtggtgcagg

SEQ ID NO: 296 - DNA, 22 bp, synthetic construct
  source 1..22: mol_type = other DNA
tgtaactcaaagtgctgctggc

SEQ ID NO: 297 - DNA, 16 bp, synthetic construct
  source 1..16: mol_type = other DNA
gccgacgtggtgccgt

SEQ ID NO: 298 - DNA, 18 bp, synthetic construct
  source 1..18: mol_type = other DNA
atggggtggctggggtag

SEQ ID NO: 299 - DNA, 20 bp, synthetic construct
  source 1..20: mol_type = other DNA
tcaacacggagctgtagggc

SEQ ID NO: 300 - DNA, 20 bp, synthetic construct
  source 1..20: mol_type = other DNA
gttgcccagcgagttctgag

SEQ ID NO: 301 - DNA, 20 bp, synthetic construct
  source 1..20: mol_type = other DNA
gcggtggagaaggagtggtt

SEQ ID NO: 302 - DNA, 17 bp, synthetic construct
  source 1..17: mol_type = other DNA
ccacgctcaggccgttg

SEQ ID NO: 303 - DNA, 17 bp, synthetic construct
  source 1..17: mol_type = other DNA
cccgttgaccagccggt

SEQ ID NO: 304 - DNA, 18 bp, synthetic construct
  source 1..18: mol_type = other DNA
cgtggcgtacgggatctc

SEQ ID NO: 305 - DNA, 17 bp, synthetic construct
  source 1..17: mol_type = other DNA
gcggccgtgaggtggtg

SEQ ID NO: 306 - DNA, 15 bp, synthetic construct
  source 1..15: mol_type = other DNA
gaggcggctagcgcg

SEQ ID NO: 307 - DNA, 16 bp, synthetic construct
  source 1..16: mol_type = other DNA
caggccgcagggcacc

SEQ ID NO: 308 - DNA, 17 bp, synthetic construct
  source 1..17: mol_type = other DNA
ccagagcagggcaccga

SEQ ID NO: 309 - DNA, 21 bp, synthetic construct
  source 1..21: mol_type = other DNA
caggggttgagggagtaggtc

SEQ ID NO: 310 - DNA, 18 bp, synthetic construct
  source 1..18: mol_type = other DNA
gcgagcaggttgacggag

SEQ ID NO: 311 - DNA, 22 bp, synthetic construct
  source 1..22: mol_type = other DNA
gaaaaagtaactggtctggccc

SEQ ID NO: 312 - DNA, 16 bp, synthetic construct
  source 1..16: mol_type = other DNA
ggtgcgggacgtgggg

SEQ ID NO: 313 - DNA, 21 bp, synthetic construct
  source 1..21: mol_type = other DNA
tgctctgcgaagtcattgacg

SEQ ID NO: 314 - DNA, 17 bp, synthetic construct
  source 1..17: mol_type = other DNA
ggcgctcatggacgtgc

SEQ ID NO: 315 - DNA, 16 bp, synthetic construct
  source 1..16: mol_type = other DNA
aggaggacgcggccct

(19) United States
(12) Patent Application Publication
(10) Pub. No.: US 2026/0308582 A1
(43) Pub. Date: Oct. 8, 2026

(54) OPTIMIZED GAPMERS ANTISENSE OLIGONUCLEOTIDES FOR INCREASING FOXG1 EXPRESSION

(71) Applicant: ELIGAB TX LLC, Manhasset, NY (US)

(72) Inventors: Scott REICH, Port Washington, NY (US); Brian BETTENCOURT, Groton, MA (US)

(21) Appl. No.: 19/100,908

(22) PCT Filed: Aug. 4, 2023
(86) PCT No.: PCT/US2023/029553

§ 371 Date: Feb. 3, 2025

Related U.S. Application Data
(60) Provisional application No. 63/370,871, filed on Aug. 9, 2022.

(51) Int. Cl. C12N 15/113

(52) U.S. Cl. CPC C12N 15/113; C12N 2310/113; C12N 2310/315; C12N 2310/341

(57) ABSTRACT

Provided herein are compositions and methods for treating and/or ameliorating the FOXG1 syndrome or the symptoms associated therewith. The compositions and methods disclosed herein utilize antisense oligonucleotides that target long non-coding RNAs (lncRNAs) to increase FOXG1 expression in a cell, thereby restoring FOXG1 function.

### CROSS-REFERENCE

This application claims the benefit of U.S. Provisional Patent Application No. 63/370,871, filed Aug. 9, 2022, which is incorporated herein by reference in its entirety.

### BACKGROUND

FOXG1 syndrome is a rare neurodevelopmental disorder associated with heterozygous variants in the forkhead box G1 (FOXG1) gene and is characterized by impaired neurological development and/or altered brain physiology. Observed phenotypes of FOXG1 syndrome primarily include a particular pattern of structural alterations in the brain resulting from inherited de novo mutations in the FOXG1 gene. Such structural alterations include a thin or underdeveloped corpus callosum that connects between the right and left hemispheres of the brain, reduced sulci and gyri formation on the surface of the brain, and/or a reduced amount of white matter. Currently, there is no therapeutic modality for treating and/or preventing FOXG1 syndrome.

### SUMMARY

Provided herein are compositions and methods for treating and/or ameliorating FOXG1 syndrome or the symptoms associated therewith. The compositions and methods described herein utilize antisense oligonucleotides that target long non-coding RNAs (lncRNAs) to increase FOXG1 expression. In certain instances, the targeted long non-coding RNAs (lncRNAs) down regulate FOXG1 expression (e.g., mRNA or protein), wherein the gapmer antisense oligonucleotides (ASOs) thereby prevent or inhibit or reduce lncRNA-mediated down-regulation of FOXG1 expression. The ability to restore or increase functional FOXG1 expression in cells provides a foundation for the treatment of FOXG1 syndrome or alleviating symptoms associated therewith. The compositions and methods described herein are, in part, based on the discovery that FOXG1 expression can be increased by targeting long non-coding RNAs (lncRNAs) with antisense oligonucleotides. Accordingly, the present disclosure (i) provides that FOXG1 expression can be increased by targeting long non-coding RNAs (lncRNAs) with antisense oligonucleotides, and (ii) provides assays and methods for the identification of antisense oligonucleotides that increase FOXG1 expression by targeting long non-coding RNAs (lncRNAs).

Provided herein are gapmer antisense oligonucleotides (ASOs), comprising a sequence that hybridizes to a target nucleic acid sequence of a long non-coding RNA (lncRNA). In some embodiments, the lncRNA regulates expression of FOXG1. In some embodiments, the lncRNA reduces expression of FOXG1 messenger RNA. In some embodiments, the lncRNA reduces transcription of FOXG1 messenger RNA molecule. In some embodiments, the lncRNA reduces expression of FOXG1 protein. In some embodiments, the lncRNA reduces translation of a FOXG1 protein molecule.

Provided herein are gapmer antisense oligonucleotides comprising a sequence that hybridizes to a target nucleic acid sequence of a long non-coding RNA (lncRNA), wherein the gapmer comprises a structure having the formula:

X—Y—Z
wherein:

X is a 5′ wing region,

Y is a gap region, and

Z is a 3′ wing region; and

wherein X, Y, and/or Z comprise one or more modifications.

In some embodiments, the lncRNA regulates expression of FOXG1. In some embodiments, the lncRNA reduces expression of FOXG1 messenger RNA. In some embodiments, the lncRNA reduces transcription of FOXG1 messenger RNA molecule.

In some embodiments, the lncRNA reduces expression of FOXG1 protein. In some embodiments, the lncRNA reduces translation of a FOXG1 protein molecule.

In some embodiments, X comprises one or more nucleoside modifications. In some embodiments, Z comprises one or more nucleoside modifications. In some embodiments, X and Z each comprise one or more nucleoside modifications. In some embodiments, the one or more nucleoside modifications comprises a LNA (2′-4′ biradicle bridged) nucleoside, 2′ substituted nucleoside (e.g., 2′-O-alkyl-RNA, 2′-O-methyl-RNA, 2′-alkoxy-RNA, 2′-O-methoxyethyl-oligos (MOE), 2′-amino-DNA, 2′-Fluoro-RNA, and 2′-F-ANA nucleosides), nucleobase modifications. Or a combination thereof.

In some embodiments, X comprises one or more inter-nucleoside linker modifications. In some embodiments, Y comprises one or more inter-nucleoside linker modifications. In some embodiments, Z comprises one or more inter-nucleoside linker modifications. In some embodiments, X and Z each comprise one or more inter-nucleoside linker modifications. In some embodiments, X, Y, and Z each comprise one or more inter-nucleoside linker modifications.

In some embodiments, the sequence is complementary to the target nucleic acid sequence of a long non-coding RNA (lncRNA). In some embodiments, the long non-coding RNA (lncRNA) is located within 1 kilobases (kb), 2 kb, 5 kb, 8 kb, or 10 kb of a gene encoding FOXG1. In some embodiments, the long non-coding RNA (lncRNA) is FOXG1-AS1, long intergenic non-protein coding RNA 1551, long intergenic non-protein coding RNA 2282 (LINC02282), or a combination thereof. In some embodiments, the sequence comprises a nucleobase sequence as set forth in any one of Table 3. In some embodiments, the gapmer antisense oligonucleotide comprises a nucleobase sequence as set forth in any one of Table 4. In some embodiments, the gapmer antisense oligonucleotide hybridizes to the target nucleic acid sequence comprising or adjacent to any one or more of the positions provided in Table 3. In some embodiments, adjacent to any one or more of the positions provided in Table 3 comprises base positions within 20, 40, 50, 75, 100, or 150 base positions 5′ and/or 3′. In some embodiments, the gapmer antisense oligonucleotide hybridizes to the target nucleic acid sequence comprising or adjacent to any one or more of the positions provided in Table 4, In some embodiments, adjacent to any one or more of the positions provided in Table 4 comprises base positions within 20, 40, 50, 75, 100, or 150 base positions 5′ and/or 3′.

In some embodiments, hybridization of the sequence of the gapmer antisense oligonucleotide to the target nucleic acid sequence increases degradation of the lncRNA. In some embodiments, hybridization of the sequence of the gapmer antisense oligonucleotide to the target nucleic acid sequence increases expression of FOXG1. In some embodiments, expression of FOXG1 is mRNA expression. In some embodiments, expression of FOXG1 is protein expression.

Also provided are methods of modulating expression of FOXG1 in a cell, comprising contacting the cell with the gapmer antisense oligonucleotide of any one of the preceding embodiments. In some embodiments, the cell is a located in a brain of an individual. In some embodiments, the individual is a human. In some embodiments, the individual comprises reduced FOXG1 expression or a FOXG1 deficiency. In some embodiments, the individual has a FOXG1 disease or disorder.

In some embodiments, the FOXG1 disease or disorder is FOXG1 syndrome. In some embodiments, modulating expression comprises increasing expression of a FOXG1 protein in the cell. In some embodiments, modulating expression comprises increasing translation of a FOXG1 protein in the cell.

Further provided are methods of treating or ameliorating a FOXG1 disease or disorder in an individual having, or at risk of having, the FOXG1 disease or disorder, comprising administering to the individual the gapmer antisense oligonucleotide of any one of the preceding embodiments. In some embodiments, the individual is a human. In some embodiments, the human is an unborn human. In some embodiments, the individual comprises a mutated FOXG1 gene, a reduced expression of FOXG1, a deficiency of FOXG1, or a combination thereof. In some embodiments, the FOXG1 disease or disorder is FOXG1 syndrome.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a diagram of a FOXG1 transcript.

FIGS. 2A, 2B, 2C, 2D, and 2E show gapmer antisense oligonucleotides (ASOs) that target long non-coding RNAs and increase FOXG1 expression.

### DETAILED DESCRIPTION

Deletions or mutations in a single allele of the forkhead box G1 (FOXG1) gene cause FOXG1 syndrome. FOXG1 syndrome is a rare disease characterized by developmental delay, severe intellectual disability, epilepsy, absent language, and dyskinesis. Hallmarks of altered brain physiologies associated with FOXG1 syndrome include cortical atrophy and agenesis of the corpus callosum. The FOXG1 gene/protein is a member of the forkhead transcription factor family and is expressed specifically in neural progenitor cells of the forebrain. The FOXG1 gene is composed of one coding exon and notably, the location or type of FOXG1 mutation can be associated with or indicative of clinical severity.

The FOXG1 protein plays an important role in brain development, particularly in a region of the embryonic brain known as the telencephalon. The telencephalon ultimately develops into several critical structures, including the largest part of the brain (i.e. cerebrum), which controls most voluntary activity, language, sensory perception, learning, and memory. A shortage of functional FOXG1 protein, as observed in individuals having mutations or deletions in a single FOXG1 allele (i.e. heterozygous individuals), disrupts normal brain patterning and development.

Expression of a target get can regulated by long non-coding ribonucleic acids (lncRNAs) at multiple levels. For example, by interacting with DNA, RNA, and proteins, lncRNAs can modulate the transcription of neighboring and distant genes, and affect RNA splicing, stability and translation.

Described herein are compositions and methods of modulating the status, activity, or expression of long intervening (which includes both intronic and intergenic) non-coding RNAs (lncRNAs) in a cell, tissue or organism. Also provided are compositions and methods for treating pathological conditions and diseases in a mammal caused by or modulated by the regulatory, structural, catalytic or signaling properties of a lncRNA. Accordingly, disclosed herein are compositions and methods useful for increasing an amount of functional FOXG1 (e.g., FOXG1 protein or FOXG1 messenger ribonucleic acid (mRNA)) in a cell having a shortage of functional FOXG1. Such compositions and methods are useful in their application for treating individual having a FOXG1-related disease or disorder wherein the lack or shortage of functional FOXG1 protein can be remedied. In order to achieve an increase of FOXG1 expression in cells or in an individual, antisense oligonucleotides targeting lncRNAs are used.

### Antisense Oligonucleotides

Antisense oligonucleotides (ASOs) are small (~18-30 nucleotides), synthetic, single-stranded nucleic acid polymers that can be employed to modulate gene expression by various mechanisms. Antisense oligonucleotides (ASOs) can be subdivided into two major categories: RNase H competent and steric block. For RNase H competent antisense oligonucleotides (e.g., gapmer ASOs), the endogenous RNase H enzyme recognizes RNA-DNA heteroduplex substrates that are formed when antisense oligonucleotides bind to their cognate mRNA transcripts to catalyze the degradation of RNA.

In order to achieve effective targeting of a lncRNA, the antisense oligonucleotides (e.g., gapmer ASOs) described herein hybridize to a target nucleic acid sequence of a long non-coding RNA (lncRNA). In certain instances, a lncRNA generally can be defined as an RNA molecule having greater than about 200 nucleotides, wherein the RNA molecule does not encode for a protein sequence or translated protein sequence or translatable protein sequence. In certain instances, the lncRNA is transcribed from an intergene region or intraintronic region. In some embodiments, the lncRNA comprises greater than about 200 kilobases (kb), 400 kb, 500 kb, 1000 kb, or 2000 kb.

lncRNAs can regulate FOXG1 through one or more various or different mechanisms. In some embodiments, the lncRNA reduces expression of FOXG1 messenger RNA. In some embodiments, the lncRNA reduces transcription of FOXG1 messenger RNA molecule. In some embodiments, the lncRNA reduces expression of FOXG1 protein. In some embodiments, the lncRNA reduces translation of a FOXG1 protein molecule.

Targeting (e.g., hybridization) to a lncRNA, in some embodiments, is achieved by modified antisense oligonucleotides (e.g., gapmer ASOs) comprising a sequence complementary or substantially complementary (e.g., having at least 70%, 80%, 90%, 95%, or 100% sequence identity) to a target nucleic acid sequence of a long non-coding RNA (lncRNA). In some embodiments, the sequence is complementary to the target nucleic acid sequence of a long non-coding RNA (lncRNA). In some embodiments, the long non-coding RNA (lncRNA) is FOXG1-AS1, long non-protein coding RNA 1551 (LINC01151), long intergenic non-protein coding RNA 2282 (LINC02282), or a combination thereof. In some embodiments, the sequence comprises a nucleobase sequence as set forth in any one of SEQ ID NOs: 1-288.

In some embodiments, the sequence is complementary to the target nucleic acid sequence at or adjacent (e.g., surrounding) to positions 200-350 or 375-525 of long non-coding RNA (lncRNA) FOXG1-AS1 (e.g., reference sequence NR_125758.1). In some embodiments, the antisense oligonucleotide hybridizes to a target nucleic acid sequence at or adjacent (e.g., surrounding) to positions 200-350 or 375-525 of long non-coding RNA (lncRNA) FOXG1-AS1. In certain embodiments, the antisense oligonucleotide hybridizes to a target nucleic acid sequence at or adjacent (e.g., surrounding) to the positions provided in Table 3 or 4 for long non-coding RNA (lncRNA) FOXG1-AS1. In certain embodiments, the antisense oligonucleotide hybridizes to a target nucleic acid sequence adjacent (e.g., surrounding) to the positions provided in Table 3 for long non-coding RNA (lncRNA) FOXG1-AS1. In certain embodiments, adjacent to or surrounding includes base positions within 20, 40, 50, 75, 100, or 150 base positions 5′ and/or 3′. In certain embodiments, the antisense oligonucleotide hybridizes to a target nucleic acid sequence adjacent (e.g., surrounding) to the positions provided in Table 4 for long non-coding RNA (lncRNA) FOXG1-AS1. In certain embodiments, adjacent to or surrounding includes base positions within 20, 40, 50, 75, 100, or 150 base positions 5′ and/or 3′. In certain embodiments, the adjacent to or surrounding base positions are within 20 base positions 5′ and/or 3′. In certain embodiments, the adjacent to or surrounding base positions are within 40 base positions 5′ and/or 3′. In certain embodiments, the adjacent to or surrounding base positions are within 50 base positions 5′ and/or 3′. In certain embodiments, the adjacent to or surrounding base positions are within 75 base positions 5′ and/or 3′. In certain embodiments, the adjacent to or surrounding base positions are within 100 base positions 5′ and/or 3′. In certain embodiments, the adjacent to or surrounding base positions are within 150 base positions 5′ and/or 3′.

In some embodiments, the sequence is complementary to the target nucleic acid sequence at or adjacent (e.g., surrounding) to positions 950-1150 or 1450-1650 or 2150-2350 or 3450-3730 of long non-coding RNA (lncRNA) LINC01551 (e.g., reference sequence NR_026732.1 and NR_026731.1-merged exons). In some embodiments, the antisense oligonucleotide hybridizes to a target nucleic acid sequence at or adjacent (e.g., surrounding) to positions 950-1150 or 1450-1650 or 2150-2350 or 3450-3730 of long non-coding RNA (lncRNA) LINC01551. In certain embodiments, the antisense oligonucleotide hybridizes to a target nucleic acid sequence at or adjacent (e.g., surrounding) to the positions provided in Table 3 or 4 for long non-coding RNA (lncRNA) LINC01551. In certain embodiments, the antisense oligonucleotide hybridizes to a target nucleic acid sequence adjacent (e.g., surrounding) to the positions provided in Table 3 for long non-coding RNA (lncRNA) LINC01551, wherein adjacent to or surrounding includes base positions within 20, 40, 50, 75, 100, or 150 base positions 5′ and/or 3′. In certain embodiments, the antisense oligonucleotide hybridizes to a target nucleic acid sequence adjacent (e.g., surrounding) to the positions provided in Table 4 for long non-coding RNA (lncRNA) LINC01551, wherein adjacent to or surrounding includes base positions within 20, 40, 50, 75, 100, or 150 base positions 5′ and/or 3′. In certain embodiments, the adjacent to or surrounding base positions are within 20 base positions 5′ and/or 3′. In certain embodiments, the adjacent to or surrounding base positions are within 40 base positions 5′ and/or 3′. In certain embodiments, the adjacent to or surrounding base positions are within 50 base positions 5′ and/or 3′. In certain embodiments, the adjacent to or surrounding base positions are within 75 base positions 5′ and/or 3′. In certain embodiments, the adjacent to or surrounding base positions are within 100 base positions 5′ and/or 3′. In certain embodiments, the adjacent to or surrounding base positions are within 150 base positions 5′ and/or 3′.

In some embodiments, the sequence is complementary to the target nucleic acid sequence at or adjacent (e.g., surrounding) to positions 100-300 or 360-560 or 730-970 or 780-1083 or 1228 to 1349 of long non-coding RNA (lncRNA) LINC02282 (e.g., reference sequence NR_026732.1 and NR_026731.1-merged exons). In some embodiments, the antisense oligonucleotide hybridizes to a target nucleic acid sequence at or adjacent (e.g., surrounding) to positions 100-300 or 360-560 or 730-970 or 780-1083 or 1228 to 1349 of long non-coding RNA (lncRNA) LINC01551. In certain embodiments, the antisense oligonucleotide hybridizes to a target nucleic acid sequence at or adjacent (e.g., surrounding) to the positions provided in Table 3 or 4 for long non-coding RNA (lncRNA) LINC02282. In certain embodiments, the antisense oligonucleotide hybridizes to a target nucleic acid sequence adjacent (e.g., surrounding) to the positions provided in Table 3 for long non-coding RNA (lncRNA) LINC02282, wherein adjacent to or surrounding includes base positions within 20, 40, 50, 75, 100, or 150 base positions 5′ and/or 3′. In certain embodiments, the antisense oligonucleotide hybridizes to a target nucleic acid sequence adjacent (e.g., surrounding) to the positions provided in Table 4 for long non-coding RNA (lncRNA) LINC02282, wherein adjacent to or surrounding includes base positions within 20, 40, 50, 75, 100, or 150 base positions 5′ and/or 3′. In certain embodiments, the adjacent to or surrounding base positions are within 20 base positions 5′ and/or 3′. In certain embodiments, the adjacent to or surrounding base positions are within 40 base positions 5′ and/or 3′. In certain embodiments, the adjacent to or surrounding base positions are within 50 base positions 5′ and/or 3′. In certain embodiments, the adjacent to or surrounding base positions are within 75 base positions 5′ and/or 3′. In certain embodiments, the adjacent to or surrounding base positions are within 100 base positions 5′ and/or 3′. In certain embodiments, the adjacent to or surrounding base positions are within 150 base positions 5′ and/or 3′.

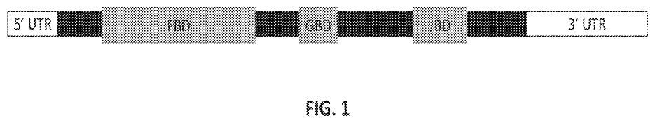

In certain instances, targeting (e.g., hybridization) to the lncRNA increases FOXG1 expression. In certain instances, targeting (e.g., hybridization) to the lncRNA prevents lncRNA-mediated down regulation of FOXG1 by promoting the degradation of the lncRNA. In certain instances, targeting (e.g., hybridization) to the lncRNA prevents lncRNA-mediated down regulation of FOXG1 by promoting the degradation of the lncRNA. Accordingly, in some embodiments, hybridization of the sequence of the antisense oligonucleotide to the target nucleic acid sequence increases degradation of the lncRNA. In some embodiments, hybridization of the sequence of the antisense oligonucleotide to the target nucleic acid sequence increases expression of FOXG1. In certain embodiments, expression of FOXG1 is mRNA expression. In certain embodiments, expression of FOXG1 is protein expression. Such ASOs are suitable for use in the methods described herein. FIG. 1 shows a diagram of the FOXG1 mRNA transcript. TABLE 1 discloses sequences and antisense oligonucleotides (ASOs) sequences for targeting to lncRNAs.

Compositions comprising one or more of the ASOs described herein are useful. In certain embodiments, combing two or more ASOs having a different sequence are used to increase FOXG1 expression in a cell. In certain embodiments, the compositions are a pharmaceutical composition.

In order to improve the pharmacodynamic, pharmacokinetic, and biodistribution properties of antisense oligonucleotides (e.g., gapmer ASOs), the antisense oligonucleotides can be designed and engineered to comprise one or more chemical modifications (e.g., a modified inter-nucleoside linker, a modified nucleoside, or a combination thereof). Accordingly, in some embodiments, the antisense oligonucleotide is a modified oligonucleotide. In some embodiments, the antisense oligonucleotide comprises one or more modifications. In certain embodiments, the modification comprises a modified inter-nucleoside linker, a modified nucleoside, or a combination thereof.

### Modified Nucleosides

Modifications to the ribose sugar or nucleobase can also be utilized to increase pharmacodynamic, pharmacokinetic, and biodistribution properties. Similar to modifications of the inter-nucleoside linker, nucleoside modifications prevent or reduce degradation by cellular nucleases, thus increasing the pharmacokinetics and bioavailability of the antisense oligonucleotide. Generally, a modified nucleoside includes the introduction of one or more modifications of the sugar moiety or the nucleobase moiety.

The antisense oligonucleotides, as described herein, can comprise one or more nucleosides comprising a modified sugar moiety, wherein the modified sugar moiety is a modification of the sugar moiety when compared to the ribose sugar moiety found in deoxyribose nucleic acid (DNA) and RNA. Numerous nucleosides with modification of the ribose sugar moiety can be utilized, primarily with the aim of improving certain properties of oligonucleotides, such as affinity and/or nuclease resistance. Such modifications include those where the ribose ring structure is modified. These modifications include replacement with a hexose ring (HNA), a bicyclic ring having a biradicle bridge between the C2 and C4 carbons on the ribose ring (e.g., locked nucleic acids (LNA)), or an unlinked ribose ring which typically lacks a bond between the C2 and C3 carbons (e.g., UNA). Other sugar modified nucleosides include, for example, bicyclohexose nucleic acids or tricyclic nucleic acids. Modified nucleosides also include nucleosides where the sugar moiety is replaced with a non-sugar moiety, for example in the case of peptide nucleic acids (PNA), or morpholino nucleic acids.

Sugar modifications also include modifications made by altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity. A 2′ sugar modified nucleoside is a nucleoside that has a substituent other than H or —OH at the 2′ position (2′ substituted nucleoside) or comprises a 2′ linked biradicle, and includes 2′ substituted nucleosides and LNA (2′-4′ biradicle bridged) nucleosides. Examples of 2′ substituted modified nucleosides are 2′-O-alkyl-RNA, 2′-O-methyl-RNA, 2′-alkoxy-RNA, 2′-O-methoxyethyl-oligos (MOE), 2′-amino-DNA, 2′-Fluoro-RNA, and 2′-F-ANA nucleoside. In some embodiments, the antisense oligonucleotide comprises one or more modified sugars. In some embodiments, the antisense oligonucleotide comprises only modified sugars. In certain embodiments, the antisense oligo comprises greater than 10%, 25%, 50%, 75%, or 90% modified sugars. In some embodiments, the modified sugar is a bicyclic sugar. In some embodiments, the modified sugar comprises a 2′-O-methoxyethyl (MOE) group.

In some embodiments, the antisense oligonucleotide comprises both inter-nucleoside linker modifications and nucleoside modifications. In some embodiments a certain region (e.g., the 5′ and/or 3′ region) or regions (e.g., the 5′ and 3′ regions) of the ASO linker modifications and nucleoside modifications. In certain embodiments, a 5′ region and 3′ region of the ASO comprise a modified linker and nucleoside modifications. In certain embodiments, a 5′ region and 3′ region of the ASO comprise a modified linker and nucleoside modifications, wherein the ASO comprises an unmodified region or segment between a 5′ modified region and 3′ modified region of the ASO.

### Modified Inter-Nucleoside Linkers

Modification of the inter-nucleoside linker (i.e. backbone) can be utilized to increase pharmacodynamic, pharmacokinetic, and biodistribution properties. For example, inter-nucleoside linker modifications prevent or reduce degradation by cellular nucleases, thus increasing the pharmacokinetics and bioavailability of the antisense oligonucleotide. Generally, a modified inter-nucleoside linker includes any linker other than other than phosphodiester (PO) liners, that covalently couples two nucleosides together. In some embodiments, the modified inter-nucleoside linker increases the nuclease resistance of the antisense oligonucleotide compared to a phosphodiester linker. For naturally occurring antisense oligonucleotides, the inter-nucleoside linker includes phosphate groups creating a phosphodiester bond between adjacent nucleosides. Modified inter-nucleoside linkers are particularly useful in stabilizing antisense oligonucleotides for in vivo use and may serve to protect against nuclease cleavage.

In some embodiments, the antisense oligonucleotide comprises one or more inter-nucleoside linkers modified from the natural phosphodiester to a linker that is for example more resistant to nuclease attack. In some embodiments a certain region (e.g., the 5′ and/or 3′ region) or regions (e.g., the 5′ and 3′ regions) of the antisense oligonucleotide, or contiguous nucleotide comprises a modified inter-nucleoside linker. In certain embodiments, a 5′ region and 3′ region of the ASO comprise a modified linker. In certain embodiments, a 5′ region and 3′ region of the ASO comprise a modified linker, wherein the ASO comprises an unmodified region or segment between a 5′ modified region and 3′ modified region of the ASO. In some embodiments all of the inter-nucleoside linkers of the antisense oligonucleotide, or contiguous nucleotide sequence thereof, are modified. In some embodiments all of the inter-nucleoside linkers of the antisense oligonucleotide, or contiguous nucleotide sequence thereof, are nuclease resistant inter-nucleoside linkers. In some embodiments the inter-nucleoside linkage comprises sulphur(S), such as a phosphorothioate inter-nucleoside linkage.

In certain instances, phosphorothioate inter-nucleoside linkers are particularly useful due to nuclease resistance and improved pharmacokinetics. In some embodiments, one or more of the inter-nucleoside linkers of the antisense oligonucleotide, or contiguous nucleotide a sequence thereof, comprise phosphorothioate inter-nucleoside linker. In some embodiments, all of the inter-nucleoside linkers of the antisense oligonucleotide, or contiguous nucleotide sequence thereof, comprise a phosphorothioate inter-nucleoside linker.

### Gapmers

Further provided herein are modified ASOs that promote degradation of a target lncRNA, wherein such ASOs can be referred to as gapmers ASOs. In certain instances, a gapmer or gapped ASO refers to an oligomeric compound having two modified external regions and an unmodified internal or central region or segment. For example, a gapmer generally refers to and encompasses a gapmer antisense oligonucleotide which comprises a region of RNase H recruiting oligonucleotides (gap) flanked 5′ and 3′ by regions which comprise one or more affinity enhancing modified nucleosides (flanks or wings). Gapmer oligonucleotides are generally used to inhibit a target RNA in a cell, such as an inhibitory lncRNA, via an antisense mechanism (and may therefore also be called antisense gapmer oligonucleotides). Gapmer oligonucleotides generally comprise a region of at least about 5 contiguous nucleotides which are capable or recruiting RNaseH (gap region), such as a region of DNA nucleotides, e.g., 6-14 DNA nucleotides, flanked 5′ and 3′ by regions which comprise affinity enhancing modified nucleosides, such as LNA or 2′ substituted nucleotides. In some embodiments, the flanking regions may be 1-8 nucleotides in length.

A high affinity modified nucleoside generally includes and refers a modified nucleotide which, when incorporated into the oligonucleotide enhances the affinity of the oligonucleotide for its complementary target, for example as measured by the melting temperature (Tm). A high affinity modified nucleoside preferably results in an increase in melting temperature between +0.5 to +12° C., more preferably between +1.5 to +10° C. and most preferably between +3 to +8° C. per modified nucleoside. High affinity modified nucleosides generally include for example, many 2′ substituted nucleosides as well as locked nucleic acids (LNA).

In some embodiments, the parent and child oligonucleotides are gapmer oligonucleotides which comprise a central region of at least 5 or more contiguous nucleosides, such as at least 5 contiguous DNA nucleosides, and a 5′ wing region comprising of 1-6 high affinity nucleoside analogues, such as LNA nucleosides and a 3′ wing region comprising of 1-6 high affinity nucleoside analogues, such as LNA 1-6 nucleosides. An LNA gapmer oligonucleotide is an oligonucleotide which comprises at least one LNA nucleoside in the wing regions, and may for example comprise at least one LNA in both the 5′ and 3′ wing regions.

For example, in some embodiments, the three regions are a contiguous sequence with the sugar moieties of the external regions being different than the sugar moieties of the internal region and wherein the sugar moiety of a particular region is essentially the same. In certain embodiments, each a particular region has the same sugar moiety. In certain instances, the sugar moieties of the external regions are the same and the gapmer is considered a symmetric gapmer. In another instance, the sugar moiety used in the 5′-external region is different from the sugar moiety used in the 3′-external region, the gapmer is an asymmetric gapmer. In certain embodiments, the external regions are each independently 1, 2, 3, 4 or about 5 nucleotide subunits and comprise non-naturally occurring sugar moieties. In further embodiments, the internal region comprising β-D-2′-deoxyribonucleosides. In certain embodiments, the external regions each, independently, comprise from 1 to about 5 nucleotides having non-naturally occurring sugar moieties and the internal region comprises from 6 to 18 unmodified nucleosides. In further embodiments, the internal region or the gap generally comprises β-D-2′-deoxyribonucleosides but can comprise non-naturally occurring sugar moieties.

In some embodiments, the gapped oligonucleotides comprise an internal region of β-D-2′-deoxyribonucleosides with one of the two external regions comprising tricyclic nucleosides as disclosed herein. In certain embodiments, the gapped oligonucleotides comprise an internal region of β-D-2′-deoxyribonucleosides with both of the external regions comprising tricyclic nucleosides as provided herein. In certain embodiments, gapped oligonucleotides are provided herein wherein all of the nucleotides comprise non-naturally occurring sugar moieties, as described herein.

Gapmer nucleobase targets are also provided in TABLE 1 that encompasses SEQ ID NOs: 1-288. In some embodiments, the ASOs or gapmers described herein promote degradation of a lncRNA molecule. In certain embodiments, the degradation is RNAse dependent (e.g., RNase H) degradation.

Also provided herein are gapmer antisense oligonucleotides comprising a sequence that hybridizes to a target nucleic acid sequence of a long non-coding RNA (lncRNA), wherein the lncRNA reduces expression of FOXG1. In some embodiments, expression of FOXG1 is measured by FOXG1 mRNA expression. In some embodiments, expression of FOXG1 is measured by FOXG1 protein expression.

In some embodiments, the gapmer oligonucleotide comprises a modification. In some embodiments, the modification comprises a modified inter-nucleoside linker, a modified nucleoside, or a combination thereof. In some embodiments, the gapmer oligonucleotide comprises the modified inter-nucleoside linkage. In some embodiments, the sequence comprises a nucleobase sequence as set forth in any one of Table 3. In some embodiments, the antisense oligonucleotide comprises a nucleobase sequence as set forth in any one of Table 4. In some embodiments, the target nucleic acid sequence comprises one or more nucleobases complementary to a sequence selected from Table 3. In some embodiments, the target nucleic acid sequence comprises one or more nucleobases within or adjacent to any one of the reference positions selected from Table 3. In some embodiments, the target nucleic acid sequence comprises one or more nucleobases complementary to a sequence selected from Table 4. In some embodiments, the target nucleic acid sequence comprises one or more nucleobases within or adjacent to any one of the reference positions selected from Table 4.

In some embodiments, hybridization of the gapmer oligonucleotide increases FOXG1 expression in a cell. In some embodiments, the FOXG1 expression is FOXG1 mRNA expression. In some embodiments, the FOXG1 mRNA expression is measured by a probe-based quantification assay. In some embodiments, the long non-coding RNA (lncRNA) is FOXG1-AS1, long intergenic non-protein coding RNA 1551, long intergenic non-protein coding RNA 2282 (LINC02282), or a combination thereof.

### Modified Wing Regions

Generally, a gapmer comprises an internal region (i.e., gap) having a plurality of nucleotides that supports RNaseH cleavage positioned between external regions having a plurality of nucleotides (i.e., wings) that are chemically distinct from the nucleosides of the internal region. In such cases, antisense oligonucleotides having a gapmer motif (i.e., gapmer antisense oligonucleotides), the gap segment generally serves as the substrate for endonuclease cleavage, while the wing segments comprise modified nucleosides often resistant to nuclease activity or having reduced sensitivity to nuclease activity. This wing-gap-wing motif is frequently described as “X—Y—Z”, where “X” represents the length of the 5′ wing region, “Y” represents the length of the gap region, and “Z” represents the length of the 3′ wing region. As used herein, a gapmer described as “X—Y—Z” has a configuration such that the gap segment is positioned immediately adjacent to each of the 5′ wing segment and the 3′ wing segment.

Targeting (e.g., hybridization) to a lncRNA by wing regions is achieved by, in some embodiments, wing regions comprising a sequence complementary or substantially complementary (e.g., having at least 70%, 80%, 90%, 95%, or 100% sequence identity) to a target nucleic acid sequence of a long non-coding RNA (lncRNA). In some embodiments, the wing regions (e.g., a 5′ and 3′ pair of wing regions) are complementary to the target nucleic acid sequence (e.g., non-contiguous when the gap region is excluded, contiguous when the gap region is considered) of a long non-coding RNA (lncRNA). In some embodiments, the long non-coding RNA (lncRNA) is FOXG1-AS1, long non-protein coding RNA 1551 (LINC01151), long intergenic non-protein coding RNA 2282 (LINC02282), or a combination thereof. In some embodiments, the sequence comprises a nucleobase sequence as set forth in any one of SEQ ID NOs: 1-288.

In some embodiments, the wing regions are complementary to the target nucleic acid sequence at or adjacent (e.g., surrounding) to positions 200-350 or 375-525 of long non-coding RNA (lncRNA) FOXG1-AS1 (e.g., reference sequence NR_125758.1). In some embodiments, the wing regions hybridize to a target nucleic acid sequence at or adjacent (e.g., surrounding) to positions 200-350 or 375-525 of long non-coding RNA (lncRNA) FOXG1-AS1. In certain embodiments, the wing regions hybridize to a target nucleic acid sequence at or adjacent (e.g., surrounding) to the positions provided in Table 3 or 4 for long non-coding RNA (lncRNA) FOXG1-AS1. In certain embodiments, the wing regions hybridize to a target nucleic acid sequence adjacent (e.g., surrounding) to the positions provided in Table 3 for long non-coding RNA (lncRNA) FOXG1-AS1, wherein adjacent to or surrounding includes base positions within 20, 40, 50, 75, 100, or 150 base positions 5′ and/or 3′. In certain embodiments, the wing regions hybridize to a target nucleic acid sequence adjacent (e.g., surrounding) to the positions provided in Table 4 for long non-coding RNA (lncRNA) FOXG1-AS1, wherein adjacent to or surrounding includes base positions within 20, 40, 50, 75, 100, or 150 base positions 5′ and/or 3′. In certain embodiments, the adjacent to or surrounding base positions are within 20 base positions 5′ and/or 3′. In certain embodiments, the adjacent to or surrounding base positions are within 40 base positions 5′ and/or 3′. In certain embodiments, the adjacent to or surrounding base positions are within 50 base positions 5′ and/or 3′. In certain embodiments, the adjacent to or surrounding base positions are within 75 base positions 5′ and/or 3′. In certain embodiments, the adjacent to or surrounding base positions are within 100 base positions 5′ and/or 3′. In certain embodiments, the adjacent to or surrounding base positions are within 150 base positions 5′ and/or 3′.

In some embodiments, the wing regions are complementary to the target nucleic acid sequence at or adjacent (e.g., surrounding) to positions 950-1150 or 1450-1650 or 2150-2350 or 3450-3730 of long non-coding RNA (lncRNA) LINC01551 (e.g., reference sequence NR_026732.1 and NR_026731.1-merged exons). In some embodiments, the wing regions hybridize to a target nucleic acid sequence at or adjacent (e.g., surrounding) to positions 950-1150 or 1450-1650 or 2150-2350 or 3450-3730 of long non-coding RNA (lncRNA) LINC01551. In certain embodiments, the wing regions hybridize to a target nucleic acid sequence at or adjacent (e.g., surrounding) to the positions provided in Table 3 or 4 for long non-coding RNA (lncRNA) LINC01551. In certain embodiments the wing regions hybridize to a target nucleic acid sequence adjacent (e.g., surrounding) to the positions provided in Table 3 for long non-coding RNA (lncRNA) LINC01551, wherein adjacent to or surrounding includes base positions within 20, 40, 50, 75, 100, or 150 base positions 5′ and/or 3′. In certain embodiments, the wing regions hybridize to a target nucleic acid sequence adjacent (e.g., surrounding) to the positions provided in Table 4 for long non-coding RNA (lncRNA) LINC01551, wherein adjacent to or surrounding includes base positions within 20, 40, 50, 75, 100, or 150 base positions 5′ and/or 3′. In certain embodiments, the adjacent to or surrounding base positions are within 20 base positions 5′ and/or 3′. In certain embodiments, the adjacent to or surrounding base positions are within 40 base positions 5′ and/or 3′. In certain embodiments, the adjacent to or surrounding base positions are within 50 base positions 5′ and/or 3′. In certain embodiments, the adjacent to or surrounding base positions are within 75 base positions 5′ and/or 3′. In certain embodiments, the adjacent to or surrounding base positions are within 100 base positions 5′ and/or 3′. In certain embodiments, the adjacent to or surrounding base positions are within 150 base positions 5′ and/or 3′.

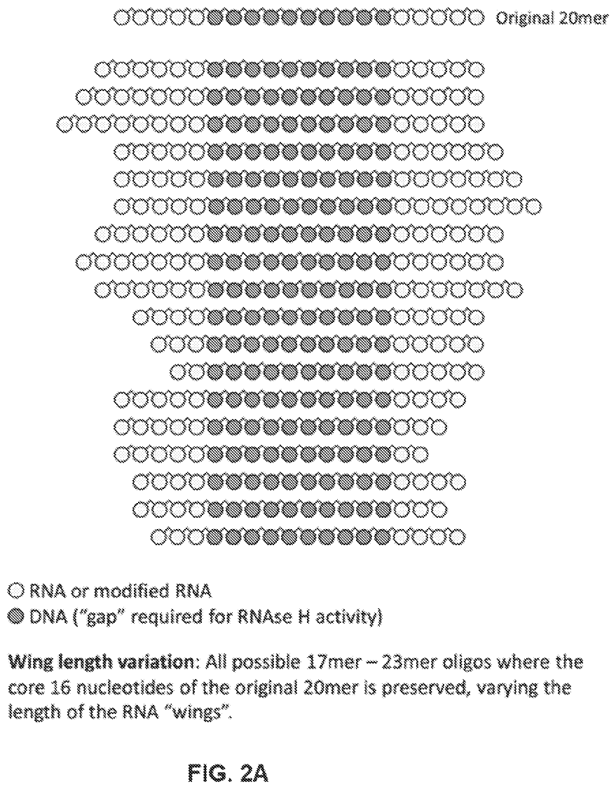

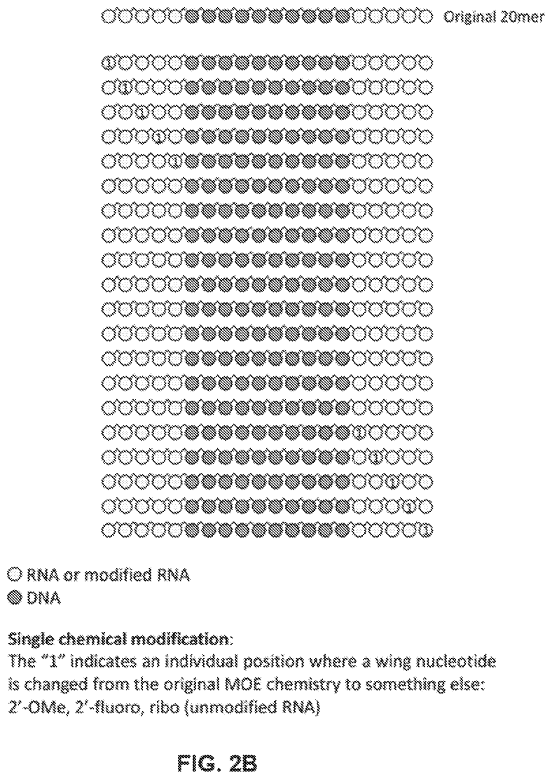

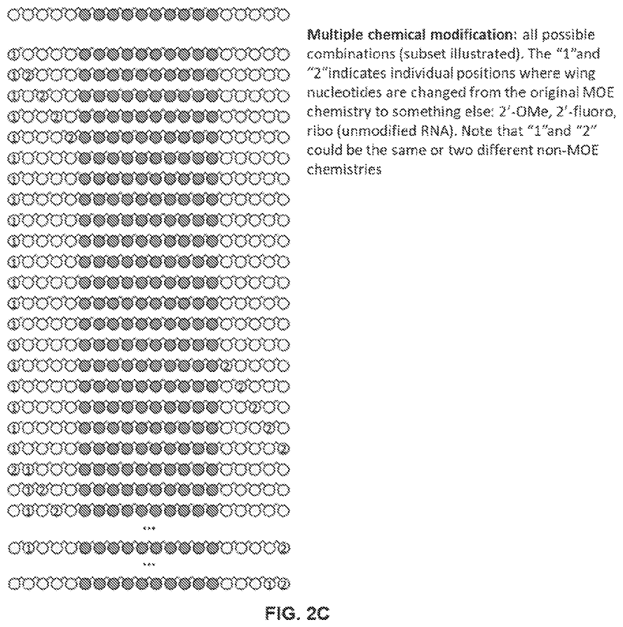

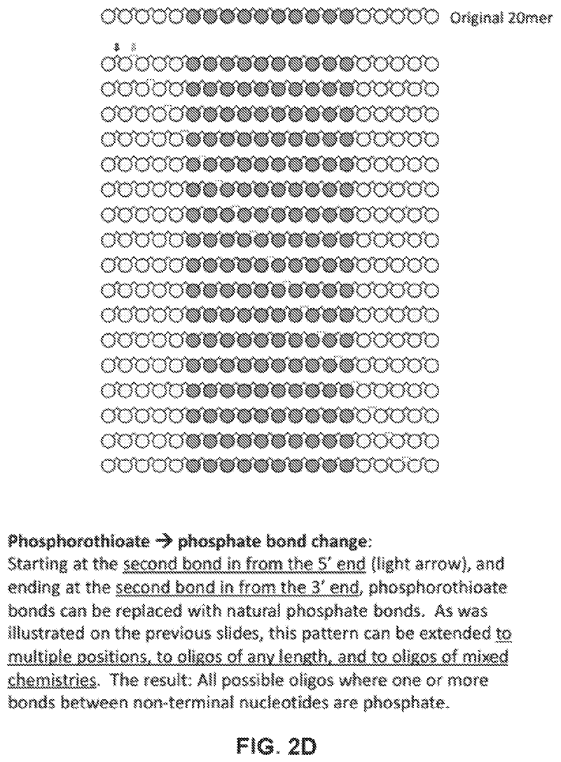

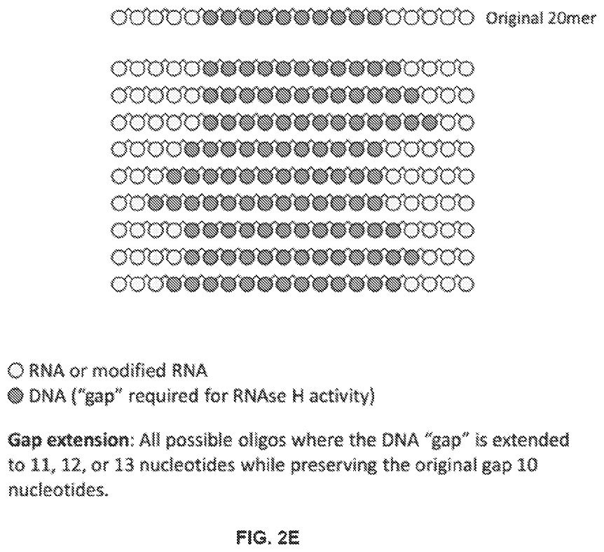

FIG. 2A shows gapmers having wing regions of varying lengths. In some embodiments, the wing regions are complementary to the target nucleic acid sequence at or adjacent (e.g., surrounding) to positions 100-300 or 360-560 or 730-970 or 780-1083 or 1228 to 1349 of long non-coding RNA (lncRNA) LINC02282 (e.g., reference sequence NR_026732.1 and NR_026731.1-merged exons). In some embodiments, the wing regions hybridize to a target nucleic acid sequence at or adjacent (e.g., surrounding) to positions 100-300 or 360-560 or 730-970 or 780-1083 or 1228 to 1349 of long non-coding RNA (lncRNA) LINC01551. In certain embodiments, the wing regions hybridize to a target nucleic acid sequence at or adjacent (e.g., surrounding) to the positions provided in Table 3 or 4 for long non-coding RNA (lncRNA) LINC02282. In certain embodiments, the wing regions hybridize to a target nucleic acid sequence adjacent (e.g., surrounding) to the positions provided in Table 3 for long non-coding RNA (lncRNA) LINC02282, wherein adjacent to or surrounding includes base positions within 20, 40, 50, 75, 100, or 150 base positions 5′ and/or 3′. In certain embodiments, the wing regions hybridize to a target nucleic acid sequence adjacent (e.g., surrounding) to the positions provided in Table 4 for long non-coding RNA (lncRNA) LINC02282, wherein adjacent to or surrounding includes base positions within 20, 40, 50, 75, 100, or 150 base positions 5′ and/or 3′. In certain embodiments, the adjacent to or surrounding base positions are within 20 base positions 5′ and/or 3′. In certain embodiments, the adjacent to or surrounding base positions are within 40 base positions 5′ and/or 3′. In certain embodiments, the adjacent to or surrounding base positions are within 50 base positions 5′ and/or 3′. In certain embodiments, the adjacent to or surrounding base positions are within 75 base positions 5′ and/or 3′. In certain embodiments, the adjacent to or surrounding base positions are within 100 base positions 5′ and/or 3′. In certain embodiments, the adjacent to or surrounding base positions are within 150 base positions 5′ and/or 3′.

In some embodiments, a wing region (e.g., a 5′ wing or 3′ wing) comprises 2 to 10 nucleobases (e.g., 2 to 10 nucleotides). In some embodiments, a wing region (e.g., a 5′ wing or 3′ wing) comprises 2 to 10 nucleobases (e.g., 2 to 10 nucleotides), wherein the total gapmer length does not exceed 25 nucleobases (e.g., 25, 24, 23, or fewer nucleobases). In some embodiments, a wing region (e.g., a 5′ wing or 3′ wing) comprises 2 to 10 nucleobases (e.g., 2 to 10 nucleotides), wherein the total gapmer length does not exceed 23 nucleobases (e.g., 23 or fewer nucleobases). In certain embodiments, a wing region (e.g., a 5′ wing or 3′ wing) comprises about 2 nucleobases to about 10 nucleobases. In certain embodiments, a wing region (e.g., a 5′ wing or 3′ wing) comprises at least about 2 nucleobases. In certain embodiments, a wing region (e.g., a 5′ wing or 3′ wing) comprises at most about 10 nucleobases. In certain embodiments, a wing region (e.g., a 5′ wing or 3′ wing) comprises about 2 nucleobases to about 3 nucleobases, about 2 nucleobases to about 4 nucleobases, about 2 nucleobases to about 5 nucleobases, about 2 nucleobases to about 6 nucleobases, about 2 nucleobases to about 7 nucleobases, about 2 nucleobases to about 8 nucleobases, about 2 nucleobases to about 9 nucleobases, about 2 nucleobases to about 10 nucleobases, about 3 nucleobases to about 4 nucleobases, about 3 nucleobases to about 5 nucleobases, about 3 nucleobases to about 6 nucleobases, about 3 nucleobases to about 7 nucleobases, about 3 nucleobases to about 8 nucleobases, about 3 nucleobases to about 9 nucleobases, about 3 nucleobases to about 10 nucleobases, about 4 nucleobases to about 5 nucleobases, about 4 nucleobases to about 6 nucleobases, about 4 nucleobases to about 7 nucleobases, about 4 nucleobases to about 8 nucleobases, about 4 nucleobases to about 9 nucleobases, about 4 nucleobases to about 10 nucleobases, about 5 nucleobases to about 6 nucleobases, about 5 nucleobases to about 7 nucleobases, about 5 nucleobases to about 8 nucleobases, about 5 nucleobases to about 9 nucleobases, about 5 nucleobases to about 10 nucleobases, about 6 nucleobases to about 7 nucleobases, about 6 nucleobases to about 8 nucleobases, about 6 nucleobases to about 9 nucleobases, about 6 nucleobases to about 10 nucleobases, about 7 nucleobases to about 8 nucleobases, about 7 nucleobases to about 9 nucleobases, about 7 nucleobases to about 10 nucleobases, about 8 nucleobases to about 9 nucleobases, about 8 nucleobases to about 10 nucleobases, or about 9 nucleobases to about 10 nucleobases. In certain embodiments, a wing region (e.g., a 5′ wing or 3′ wing) comprises about 2 nucleobases, about 3 nucleobases, about 4 nucleobases, about 5 nucleobases, about 6 nucleobases, about 7 nucleobases, about 8 nucleobases, about 9 nucleobases, or about 10 nucleobases. In some embodiments, the wing regions (e.g., X and Z) comprise the same number of nucleobases. In some embodiments, the wing regions (e.g., X and Z) comprise a different number of nucleobases.

FIG. 2B-2C show nucleobase medication schemes for gapmer antisense oligonucleotides. In some embodiments, a wing region (e.g., a 5′ wing (Y) and/or 3′ wing (Z)) comprises one or more modified nucleosides (e.g., such as those described herein). Exemplary modifications include LNA (2′-4′ biradicle bridged) nucleosides, 2′ substituted nucleosides (e.g., 2′-O-alkyl-RNA, 2′-O-methyl-RNA, 2′-alkoxy-RNA, 2′-O-methoxyethyl-oligos (MOE), 2′-amino-DNA, 2′-Fluoro-RNA, and 2′-F-ANA nucleosides), and nucleobase modifications. In certain embodiments, a wing region (e.g., a 5′ wing or 3′ wing) comprises about 1 modified nucleoside to about 10 modified nucleosides. In certain embodiments, a wing region (e.g., a 5′ wing or 3′ wing) comprises at least about 1 modified nucleoside. In certain embodiments, a wing region (e.g., a 5′ wing or 3′ wing) comprises at most about 10 modified nucleosides. In certain embodiments, a wing region (e.g., a 5′ wing or 3′ wing) comprises about 1 modified nucleoside to about 2 modified nucleosides, about 1 modified nucleoside to about 3 modified nucleosides, about 1 modified nucleoside to about 4 modified nucleosides, about 1 modified nucleoside to about 5 modified nucleosides, about 1 modified nucleoside to about 6 modified nucleosides, about 1 modified nucleoside to about 7 modified nucleosides, about 1 modified nucleoside to about 8 modified nucleosides, about 1 modified nucleoside to about 9 modified nucleosides, about 1 modified nucleoside to about 10 modified nucleosides, about 2 modified nucleosides to about 3 modified nucleosides, about 2 modified nucleosides to about 4 modified nucleosides, about 2 modified nucleosides to about 5 modified nucleosides, about 2 modified nucleosides to about 6 modified nucleosides, about 2 modified nucleosides to about 7 modified nucleosides, about 2 modified nucleosides to about 8 modified nucleosides, about 2 modified nucleosides to about 9 modified nucleosides, about 2 modified nucleosides to about 10 modified nucleosides, about 3 modified nucleosides to about 4 modified nucleosides, about 3 modified nucleosides to about 5 modified nucleosides, about 3 modified nucleosides to about 6 modified nucleosides, about 3 modified nucleosides to about 7 modified nucleosides, about 3 modified nucleosides to about 8 modified nucleosides, about 3 modified nucleosides to about 9 modified nucleosides, about 3 modified nucleosides to about 10 modified nucleosides, about 4 modified nucleosides to about 5 modified nucleosides, about 4 modified nucleosides to about 6 modified nucleosides, about 4 modified nucleosides to about 7 modified nucleosides, about 4 modified nucleosides to about 8 modified nucleosides, about 4 modified nucleosides to about 9 modified nucleosides, about 4 modified nucleosides to about 10 modified nucleosides, about 5 modified nucleosides to about 6 modified nucleosides, about 5 modified nucleosides to about 7 modified nucleosides, about 5 modified nucleosides to about 8 modified nucleosides, about 5 modified nucleosides to about 9 modified nucleosides, about 5 modified nucleosides to about 10 modified nucleosides, about 6 modified nucleosides to about 7 modified nucleosides, about 6 modified nucleosides to about 8 modified nucleosides, about 6 modified nucleosides to about 9 modified nucleosides, about 6 modified nucleosides to about 10 modified nucleosides, about 7 modified nucleosides to about 8 modified nucleosides, about 7 modified nucleosides to about 9 modified nucleosides, about 7 modified nucleosides to about 10 modified nucleosides, about 8 modified nucleosides to about 9 modified nucleosides, about 8 modified nucleosides to about 10 modified nucleosides, or about 9 modified nucleosides to about 10 modified nucleosides. In certain embodiments, a wing region (e.g., a 5′ wing or 3′ wing) comprises about 1 modified nucleoside, about 2 modified nucleosides, about 3 modified nucleosides, about 4 modified nucleosides, about 5 modified nucleosides, about 6 modified nucleosides, about 7 modified nucleosides, about 8 modified nucleosides, about 9 modified nucleosides, or about 10 modified nucleosides.

Any base position within a wing region can comprise a modified nucleoside. In some embodiments, the modifications (e.g., type of modification and/or position) within a 5′ wing and a 3′ wing are the same. In some embodiments, the modifications (e.g., type of modification and/or position) within a 5′ wing and a 3′ wing are different. Within a wing region comprising two or more modified nucleosides, the two or more modified nucleosides can be the same modification or a different modification. In some embodiments, a wing region comprises two or more modifications, wherein the two or more modifications are the same. In some embodiments, a wing region comprises two or more modifications, wherein the two or more modifications are different.

FIG. 2D shows inter-nucleoside linker modification schemes for gapmer antisense oligonucleotides. In some embodiments, a wing region (e.g., a 5′ wing (Y) and/or 3′ wing (Z)) comprises one or more modified inter-nucleoside linkers (e.g., those described herein such as PS linkages). In some embodiments, a wing region comprises one or more modified inter-nucleoside regions. In some embodiments, a wing region comprises two or more modified inter-nucleoside regions. In certain embodiments, a wing region (e.g., a 5′ wing or 3′ wing) comprises about 1 modified inter-nucleoside linker to about 10 modified inter-nucleoside linkers. In certain embodiments, a wing region (e.g., a 5′ wing or 3′ wing) comprises at least about 1 modified inter-nucleoside linker. In certain embodiments, a wing region (e.g., a 5′ wing or 3′ wing) comprises at most about 10 modified inter-nucleoside linkers. In certain embodiments, a wing region (e.g., a 5′ wing or 3′ wing) comprises about 1 modified inter-nucleoside linker to about 2 modified inter-nucleoside linkers, about 1 modified inter-nucleoside linker to about 3 modified inter-nucleoside linkers, about 1 modified inter-nucleoside linker to about 4 modified inter-nucleoside linkers, about 1 modified inter-nucleoside linker to about 5 modified inter-nucleoside linkers, about 1 modified inter-nucleoside linker to about 6 modified inter-nucleoside linkers, about 1 modified inter-nucleoside linker to about 7 modified inter-nucleoside linkers, about 1 modified inter-nucleoside linker to about 8 modified inter-nucleoside linkers, about 1 modified inter-nucleoside linker to about 9 modified inter-nucleoside linkers, about 1 modified inter-nucleoside linker to about 10 modified inter-nucleoside linkers, about 2 modified inter-nucleoside linkers to about 3 modified inter-nucleoside linkers, about 2 modified inter-nucleoside linkers to about 4 modified inter-nucleoside linkers, about 2 modified inter-nucleoside linkers to about 5 modified inter-nucleoside linkers, about 2 modified inter-nucleoside linkers to about 6 modified inter-nucleoside linkers, about 2 modified inter-nucleoside linkers to about 7 modified inter-nucleoside linkers, about 2 modified inter-nucleoside linkers to about 8 modified inter-nucleoside linkers, about 2 modified inter-nucleoside linkers to about 9 modified inter-nucleoside linkers, about 2 modified inter-nucleoside linkers to about 10 modified inter-nucleoside linkers, about 3 modified inter-nucleoside linkers to about 4 modified inter-nucleoside linkers, about 3 modified inter-nucleoside linkers to about 5 modified inter-nucleoside linkers, about 3 modified inter-nucleoside linkers to about 6 modified inter-nucleoside linkers, about 3 modified inter-nucleoside linkers to about 7 modified inter-nucleoside linkers, about 3 modified inter-nucleoside linkers to about 8 modified inter-nucleoside linkers, about 3 modified inter-nucleoside linkers to about 9 modified inter-nucleoside linkers, about 3 modified inter-nucleoside linkers to about 10 modified inter-nucleoside linkers, about 4 modified inter-nucleoside linkers to about 5 modified inter-nucleoside linkers, about 4 modified inter-nucleoside linkers to about 6 modified inter-nucleoside linkers, about 4 modified inter-nucleoside linkers to about 7 modified inter-nucleoside linkers, about 4 modified inter-nucleoside linkers to about 8 modified inter-nucleoside linkers, about 4 modified inter-nucleoside linkers to about 9 modified inter-nucleoside linkers, about 4 modified inter-nucleoside linkers to about 10 modified inter-nucleoside linkers, about 5 modified inter-nucleoside linkers to about 6 modified inter-nucleoside linkers, about 5 modified inter-nucleoside linkers to about 7 modified inter-nucleoside linkers, about 5 modified inter-nucleoside linkers to about 8 modified inter-nucleoside linkers, about 5 modified inter-nucleoside linkers to about 9 modified inter-nucleoside linkers, about 5 modified inter-nucleoside linkers to about 10 modified inter-nucleoside linkers, about 6 modified inter-nucleoside linkers to about 7 modified inter-nucleoside linkers, about 6 modified inter-nucleoside linkers to about 8 modified inter-nucleoside linkers, about 6 modified inter-nucleoside linkers to about 9 modified inter-nucleoside linkers, about 6 modified inter-nucleoside linkers to about 10 modified inter-nucleoside linkers, about 7 modified inter-nucleoside linkers to about 8 modified inter-nucleoside linkers, about 7 modified inter-nucleoside linkers to about 9 modified inter-nucleoside linkers, about 7 modified inter-nucleoside linkers to about 10 modified inter-nucleoside linkers, about 8 modified inter-nucleoside linkers to about 9 modified inter-nucleoside linkers, about 8 modified inter-nucleoside linkers to about 10 modified inter-nucleoside linkers, or about 9 modified inter-nucleoside linkers to about 10 modified inter-nucleoside linkers. In certain embodiments, a wing region (e.g., a 5′ wing or 3′ wing) comprises about 1 modified inter-nucleoside linker, about 2 modified inter-nucleoside linkers, about 3 modified inter-nucleoside linkers, about 4 modified inter-nucleoside linkers, about 5 modified inter-nucleoside linkers, about 6 modified inter-nucleoside linkers, about 7 modified inter-nucleoside linkers, about 8 modified inter-nucleoside linkers, about 9 modified inter-nucleoside linkers, or about 10 modified inter-nucleoside linkers.

Any linker position within a wing region can comprise a modified inter-nucleoside linkage. In some embodiments, the modified inter-nucleoside linker(s) (e.g., type of modification and/or position) within a 5′ wing and a 3′ wing are the same. In some embodiments, the modified inter-nucleoside linker(s) (e.g., type of modification and/or position) within a 5′ wing and a 3′ wing are different. Within a wing region comprising two or more modified inter-nucleoside linkers, the two or more modified linkers can be the same modification or a different modification. In some embodiments, a wing region comprises two or more modified inter-nucleoside linkers, wherein the two or more linker modifications are the same. In some embodiments, a wing region comprises two or more linker modifications, wherein the two or more linker modifications are different.

### Modified Gap Regions

Generally, a gap region (Y) comprises a nucleoside specifies that facilitates, promotes, activates, or is susceptible to endonuclease activity (e.g., RNase). As described herein, DNA nucleosides are useful for promoting, facilitating, and/or activating endonuclease activity upon a gapmer binding to a cognate target. Targeting (e.g., hybridization) to a lncRNA by gap regions is achieved by, in some embodiments, gap regions comprising a sequence complementary or substantially complementary (e.g., having at least 70%, 80%, 90, 95%, or 100% sequence identity) to a target nucleic acid sequence of a long non-coding RNA (lncRNA). In some embodiments, the gap regions (e.g., a 5′ and 3′ pair of gap regions) are complementary to the target nucleic acid sequence (e.g., non-contiguous when the gap region is excluded, contiguous when the gap region is considered) of a long non-coding RNA (lncRNA). In some embodiments, the long non-coding RNA (lncRNA) is FOXG1-AS1, long non-protein coding RNA 1551 (LINC01151), long intergenic non-protein coding RNA 2282 (LINC02282), or a combination thereof. In some embodiments, the sequence comprises a nucleobase sequence as set forth in any one of SEQ ID NOs: 1-288.

In some embodiments, the gap regions are complementary to the target nucleic acid sequence at or adjacent (e.g., surrounding) to positions 200-350 or 375-525 of long non-coding RNA (lncRNA) FOXG1-AS1 (e.g., reference sequence NR_125758.1). In some embodiments, the gap regions hybridize to a target nucleic acid sequence at or adjacent (e.g., surrounding) to positions 200-350 or 375-525 of long non-coding RNA (lncRNA) FOXG1-AS1. In certain embodiments, the gap regions hybridize to a target nucleic acid sequence at or adjacent (e.g., surrounding) to the positions provided in Table 3 or 4 for long non-coding RNA (lncRNA) FOXG1-AS1. In certain embodiments, the gap regions hybridize to a target nucleic acid sequence adjacent (e.g., surrounding) to the positions provided in Table 3 for long non-coding RNA (lncRNA) FOXG1-AS1, wherein adjacent to or surrounding includes base positions within 20, 40, 50, 75, 100, or 150 base positions 5′ and/or 3′. In certain embodiments, the gap regions hybridize to a target nucleic acid sequence adjacent (e.g., surrounding) to the positions provided in Table 4 for long non-coding RNA (lncRNA) FOXG1-AS1, wherein adjacent to or surrounding includes base positions within 20, 40, 50, 75, 100, or 150 base positions 5′ and/or 3′. In certain embodiments, the adjacent to or surrounding base positions are within 20 base positions 5′ and/or 3′. In certain embodiments, the adjacent to or surrounding base positions are within 40 base positions 5′ and/or 3′. In certain embodiments, the adjacent to or surrounding base positions are within 50 base positions 5′ and/or 3′. In certain embodiments, the adjacent to or surrounding base positions are within 75 base positions 5′ and/or 3′. In certain embodiments, the adjacent to or surrounding base positions are within 100 base positions 5′ and/or 3′. In certain embodiments, the adjacent to or surrounding base positions are within 150 base positions 5′ and/or 3′.

In some embodiments, the gap regions are complementary to the target nucleic acid sequence at or adjacent (e.g., surrounding) to positions 950-1150 or 1450-1650 or 2150-2350 or 3450-3730 of long non-coding RNA (lncRNA) LINC01551 (e.g., reference sequence NR_026732.1 and NR_026731.1-merged exons). In some embodiments, the gap regions hybridize to a target nucleic acid sequence at or adjacent (e.g., surrounding) to positions 950-1150 or 1450-1650 or 2150-2350 or 3450-3730 of long non-coding RNA (lncRNA) LINC01551. In certain embodiments, the gap regions hybridize to a target nucleic acid sequence at or adjacent (e.g., surrounding) to the positions provided in Table 3 or 4 for long non-coding RNA (lncRNA) LINC01551. In certain embodiments the gap regions hybridize to a target nucleic acid sequence adjacent (e.g., surrounding) to the positions provided in Table 3 for long non-coding RNA (lncRNA) LINC01551, wherein adjacent to or surrounding includes base positions within 20, 40, 50, 75, 100, or 150 base positions 5′ and/or 3′. In certain embodiments, the gap regions hybridize to a target nucleic acid sequence adjacent (e.g., surrounding) to the positions provided in Table 4 for long non-coding RNA (lncRNA) LINC01551, wherein adjacent to or surrounding includes base positions within 20, 40, 50, 75, 100, or 150 base positions 5′ and/or 3′. In certain embodiments, the adjacent to or surrounding base positions are within 20 base positions 5′ and/or 3′. In certain embodiments, the adjacent to or surrounding base positions are within 40 base positions 5′ and/or 3′. In certain embodiments, the adjacent to or surrounding base positions are within 50 base positions 5′ and/or 3′. In certain embodiments, the adjacent to or surrounding base positions are within 75 base positions 5′ and/or 3′. In certain embodiments, the adjacent to or surrounding base positions are within 100 base positions 5′ and/or 3′. In certain embodiments, the adjacent to or surrounding base positions are within 150 base positions 5′ and/or 3′.

FIG. 2E shows gapmers having DNA-based gap regions of varying lengths. In some embodiments, the gap regions are complementary to the target nucleic acid sequence at or adjacent (e.g., surrounding) to positions 100-300 or 360-560 or 730-970 or 780-1083 or 1228 to 1349 of long non-coding RNA (lncRNA) LINC02282 (e.g., reference sequence NR_026732.1 and NR_026731.1-merged exons). In some embodiments, the gap regions hybridize to a target nucleic acid sequence at or adjacent (e.g., surrounding) to positions 100-300 or 360-560 or 730-970 or 780-1083 or 1228 to 1349 of long non-coding RNA (lncRNA) LINC01551. In certain embodiments, the gap regions hybridize to a target nucleic acid sequence at or adjacent (e.g., surrounding) to the positions provided in Table 3 or 4 for long non-coding RNA (lncRNA) LINC02282. In certain embodiments, the gap regions hybridize to a target nucleic acid sequence adjacent (e.g., surrounding) to the positions provided in Table 3 for long non-coding RNA (lncRNA) LINC02282, wherein adjacent to or surrounding includes base positions within 20, 40, 50, 75, 100, or 150 base positions 5′ and/or 3′. In certain embodiments, the gap regions hybridize to a target nucleic acid sequence adjacent (e.g., surrounding) to the positions provided in Table 4 for long non-coding RNA (lncRNA) LINC02282, wherein adjacent to or surrounding includes base positions within 20, 40, 50, 75, 100, or 150 base positions 5′ and/or 3′. In certain embodiments, the adjacent to or surrounding base positions are within 20 base positions 5′ and/or 3′. In certain embodiments, the adjacent to or surrounding base positions are within 40 base positions 5′ and/or 3′. In certain embodiments, the adjacent to or surrounding base positions are within 50 base positions 5′ and/or 3′. In certain embodiments, the adjacent to or surrounding base positions are within 75 base positions 5′ and/or 3′. In certain embodiments, the adjacent to or surrounding base positions are within 100 base positions 5′ and/or 3′. In certain embodiments, the adjacent to or surrounding base positions are within 150 base positions 5′ and/or 3′.

In some embodiments, a gap region comprises 10 to 15 nucleobases. In some embodiments, a gap region comprises 10 to 15 nucleobases, wherein the total gapmer length (wings and gap region) does not exceed about 25 nucleobases (e.g., 25, 24, 23, or fewer nucleobases). In some embodiments, a gap region comprises 10 to 15 nucleobases, wherein the total gapmer length (wings and gap region) does not exceed 25 nucleobases (e.g., 23 or fewer nucleobases). In certain embodiments, a gap region comprises about 10 nucleobases to about 15 nucleobases. In certain embodiments, a gap region comprises at least about 10 nucleobases. In certain embodiments, a gap region comprises at most about 15 nucleobases. In certain embodiments, a gap region comprises about 10 nucleobases to about 11 nucleobases, about 10 nucleobases to about 12 nucleobases, about 10 nucleobases to about 13 nucleobases, about 10 nucleobases to about 14 nucleobases, about 10 nucleobases to about 15 nucleobases, about 11 nucleobases to about 12 nucleobases, about 11 nucleobases to about 13 nucleobases, about 11 nucleobases to about 14 nucleobases, about 11 nucleobases to about 15 nucleobases, about 12 nucleobases to about 13 nucleobases, about 12 nucleobases to about 14 nucleobases, about 12 nucleobases to about 15 nucleobases, about 13 nucleobases to about 14 nucleobases, about 13 nucleobases to about 15 nucleobases, or about 14 nucleobases to about 15 nucleobases. In certain embodiments, a gap region comprises about 10 nucleobases, about 11 nucleobases, about 12 nucleobases, about 13 nucleobases, about 14 nucleobases, or about 15 nucleobases.

A linker position within a gap region can comprise a modified inter-nucleoside linkage. Within a gap region comprising two or more modified inter-nucleoside linkers, the two or more modified linkers can be the same modification or a different modification. In some embodiments, a gap region comprises two or more modified inter-nucleoside linkers, wherein the two or more linker modifications are the same. In some embodiments, a gap region comprises two or more linker modifications, wherein the two or more linker modifications are different.

### Pharmaceutical Compositions

Further provided herein are pharmaceutical compositions comprising any of the disclosed antisense oligonucleotides and a pharmaceutically acceptable diluent, carrier, salt, and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution. In some embodiments, the oligonucleotide, as described, is administered at a dose of 10-1000 μg.

The gapmer antisense oligonucleotides or oligonucleotide conjugates of the disclosure may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

### Methods of Use

The gapmer antisense oligonucleotides (ASOs) provided herein are useful for targeting a lncRNA, wherein a gapmer antisense oligonucleotide increases FOXG1 expression in a cell (e.g., expression of a functional FOXG1 mRNA and/or protein). The gapmer antisense oligonucleotides targeting a lncRNAs, as described herein, are further useful in methods for increasing the expression and/or amount of functional FOXG1 in a cell (e.g., an amount of functional FOXG1 mRNA or protein). Accordingly, provided herein are methods of modulating expression of FOXG1 in a cell, comprising contacting the cell with a composition comprising a gapmer antisense oligonucleotide that hybridizes to a target nucleic acid sequence of a long non-coding RNA (lncRNA).

Further provided, are methods of treating or ameliorating a FOXG1 disease or disorder in an individual having, or at risk of having, the FOXG1 disease or disorder, comprising administering to the individual a gapmer antisense oligonucleotide, wherein the antisense oligonucleotide comprises a sequence that hybridizes to a target nucleic acid sequence of a long non-coding RNA (lncRNA).

Generally, cells of interest include neuronal cells and/or cells associated with the brain or brain development. In some embodiments, the cell is located in a brain of an individual. In some embodiments, the cell is a neural cell. In certain embodiments, the cell is a neuron, astrocyte, or fibroblast. In some embodiments, the individual is a human. In certain embodiments, the human is an unborn human. In some embodiments, the cell and/or individual comprises a mutated FOXG1 gene, reduced FOXG1 expression, or a FOXG1 deficiency. In some embodiments the individual has been diagnosed with or at risk of a FOXG1 disease or disorder. In some embodiments the FOXG1 disease or disorder is FOXG1 syndrome.

In some embodiments, the antisense oligonucleotide comprises a sequence that is complementary to the target nucleic acid sequence of a long non-coding RNA (lncRNA). In some embodiments, the long non-coding RNA (lncRNA) is located within 1 kilobases (kb), 2 kb, 5 kb, 8 kb, or 10 kb of a gene encoding FOXG1. In some embodiments, the long non-coding RNA (lncRNA) is FOXG1-AS1, long non-protein coding RNA 1551 (LINC01151), long intergenic non-protein coding RNA 2282 (LINC02282), or a combination thereof. In some embodiments, the antisense oligonucleotide comprises a nucleobase sequence as set forth in any one of SEQ ID NOs: 1-288. In some embodiments, hybridization of the antisense oligonucleotide to the target nucleic acid sequence increases degradation of the lncRNA. In some embodiments, hybridization of the antisense oligonucleotide to the target nucleic acid sequence increases expression of FOXG1 in a cell. In certain embodiments, expression of FOXG1 is mRNA expression. In certain embodiments, expression of FOXG1 is protein expression.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., Goodman and Gilman's The Pharmacological Basis of Therapeutics, McGraw-Hill, New York, N.Y., 2001; Gennaro, Remington: The Science and Practice of Pharmacy, Lippincott, Williams, and Wilkins, New York, N.Y., 2000; Avis, et al. (eds.), Pharmaceutical Dosage Forms: Parenteral Medications, Marcel Dekker, NY, 1993; Lieberman, et al. (eds.), Pharmaceutical Dosage Forms: Tablets, Marcel Dekker, NY, 1990; Lieberman, et al. (eds.) Pharmaceutical Dosage Forms: Disperse Systems, Inc., New York, N.Y., 2000).

Compositions comprising antisense oligonucleotides (ASOs), as disclosed herein, can be provided by doses at intervals of, e.g., one day, one week, or 1-7 times per week. A specific dose protocol is one involving the maximal dose or dose frequency that avoids significant undesirable side effects.

The disclosed antisense oligonucleotides or pharmaceutical compositions thereof can be administered topically (such as, to the skin, inhalation, ophthalmic, or otic) or enterally (such as, orally or through the gastrointestinal tract) or parenterally (such as, intravenous, subcutaneous, intra-muscular, intracerebral, intracerebroventricular, or intrathecal). In some embodiments the antisense oligonucleotide or pharmaceutical compositions thereof are administered by a parenteral route including intravenous, intraarterial, subcutaneous, intraperitoneal or intramuscular injection or infusion, intrathecal or intracranial, e.g., intracerebral or intraventricular, administration. In some embodiments the active oligonucleotide or oligonucleotide conjugate is administered intravenously.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “FOXG1,” as used herein, generally refers to the gene and gene products that encode a member of the fork-head transcription factor family. The encoded protein, which functions as a transcriptional repressor, is highly expressed in neural tissues during brain development. Mutations at this locus have been associated with Rett syndrome and a diverse spectrum of neurodevelopmental disorders defined as part of the FOXG1 syndrome. Depending the context of its use, “FOXG1” can refer to the FOXG1 gene, a FOXG1 deoxyribonucleic acid molecule (DNA), a FOXG1 ribonucleic acid molecule (RNA), or a FOXG1 protein. The mRNA sequence of FOXG1 is described in “NM_005249.5→NP_005240.3 forkhead box protein G1” or “accession number NM_005249.5” or the mRNA encoded by “NCBI GENE ID: 2290”. A functional FOXG1 protein describes the wild-type or unmutated FOXG1 gene, mRNA, and/or protein. Generally, “FOXG1” refers to a functional ‘FOXG1” gene or gene product, having normal function/activity within a cell. Deletions or mutations or variants of FOXG1 are indicative of non-functional FOXG1 variants having reduced, inhibited, or ablated FOXG1 function. As disclosed herein, the compositions and methods disclosed herein are primarily concerned with modulating or increasing or restoring an amount of FOXG1 (i.e. functional FOXG1) in a cell and/or individual.

The term “oligonucleotide,” as used herein, generally refers to the as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the disclosure is man-made, and is chemically synthesized, and is typically purified or isolated. The oligonucleotide disclosed may comprise one or more modified nucleosides or nucleotides.

The term “antisense oligonucleotide,” as used herein, refers to oligonucleotides capable of modulating expression of a target gene by hybridizing to a target nucleic acid, in particular to a contiguous sequence on a target nucleic acid. Preferably, the gapmer antisense oligonucleotides of the present disclosure are single stranded. In some embodiments, the antisense oligonucleotide is single stranded.

The term modified oligonucleotide refers to an oligonucleotide comprising one or more sugar-modified nucleosides, modified nucleobases, and/or modified inter-nucleoside linkers.

The term “modified nucleoside” or “nucleoside modification,” as used herein, refers to nucleosides modified as compared to the equivalent DNA or RNA nucleoside by the introduction of one or more modifications of the sugar moiety or the (nucleo) base moiety. In some embodiments, the modified nucleoside comprise a modified sugar moiety. The term modified nucleoside may also be used herein interchangeably with the term “nucleoside analogue” or modified “units” or modified “monomers”.

The term “modified inter-nucleoside linkage” is refers to linkers other than phosphodiester (PO) linkers, that covalently couples two nucleosides together. Nucleotides with modified inter-nucleoside linkage are also termed “modified nucleotides”. In some embodiments, the modified inter-nucleoside linkage increases the nuclease resistance of the oligonucleotide compared to a phosphodiester linkage. For naturally occurring oligonucleotides, the inter-nucleoside linkage includes phosphate groups creating a phosphodiester bond between adjacent nucleosides. Modified inter-nucleoside linkers are particularly useful in stabilizing oligonucleotides for in vivo use and may serve to protect against nuclease cleavage at regions of DNA or RNA nucleosides.

The term “nucleobase” includes the purine (e.g., adenine and guanine) and pyrimidine (e.g., uracil, thymine and cytosine) moiety present in nucleosides and nucleotides which form hydrogen bonds in nucleic acid hybridization. The term nucleobase also encompasses modified nucleobases which may differ from naturally occurring nucleobases but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants.

A nucleobase moiety can be modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g., A, T, G, C or U, wherein each letter may optionally include modified nucleobases of equivalent function. For example, in the exemplified oligonucleotides, the nucleobase moieties are selected from A, T, G, C, and 5-methyl cytosine. In some embodiments, the cytosine nucleobases in a 5′cg3′ motif is 5-methyl cytosine.

The term “hybridizing” or “hybridizes” or “targets” or “binds” describes two nucleic acid strands (e.g., an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid.

The oligonucleotide comprises a contiguous nucleotide region which is complementary to or hybridizes to a sub-sequence or region of the target nucleic acid molecule. The term “target sequence” as used herein refers to a sequence of nucleotides present in the target nucleic acid which comprises the nucleobase sequence which is complementary to the contiguous nucleotide region or sequence of the oligonucleotide of the disclosure. In some embodiments, the target sequence consists of a region on the target nucleic acid which is complementary to the contiguous nucleotide region or sequence of the oligonucleotide of the present disclosure. In some embodiments the target sequence is longer than the complementary sequence of a single oligonucleotide, and may, for example represent a preferred region of the target nucleic acid which may be targeted by several oligonucleotides of the present disclosure.

In some instances, the oligonucleotide comprises a contiguous nucleotide region of at least 10 nucleotides which is complementary to or hybridizes to a target sequence present in the target nucleic acid molecule. In some instances, the contiguous nucleotide region (and therefore the target sequence) comprises of at least 10 contiguous nucleotides, such as 11, 12, 13, 14, 15, 16, 17, 18, 19, 20, 21, 22, 23, 24, 25, 30 contiguous nucleotides, such as from 15-30, such as from 18-23 contiguous nucleotides.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “a therapeutically effective amount” of a compound of the present application refers to an amount of the compound of the present application that will elicit the biological or medical response of a subject, for example, reduction or inhibition of tumor cell proliferation, or ameliorate symptoms, alleviate conditions, slow or delay disease progression, or prevent a disease, etc. In one non-limiting embodiment, the term “a therapeutically effective amount” refers to the amount of a compound of the present application that, when administered to a subject, is effective to at least partially alleviate, inhibit, prevent and/or ameliorate a condition, or a disorder or a disease, or at least partially inhibit activity of a targeted enzyme or receptor.

As used in the specification and claims, the singular forms “a”, “an” and “the” include plural references unless the context clearly dictates otherwise. For example, the term “a sample” includes a plurality of samples, including mixtures thereof.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “a therapeutically effective amount” of a compound of the present application refers to an amount of the compound of the present application that will elicit the biological or medical response of a subject, for example, reduction or inhibition of tumor cell proliferation, or ameliorate symptoms, alleviate conditions, slow or delay disease progression, or prevent a disease, etc. In one non-limiting embodiment, the term “a therapeutically effective amount” refers to the amount of a compound of the present application that, when administered to a subject, is effective to at least partially alleviate, inhibit, prevent and/or ameliorate a condition, or a disorder or a disease, or at least partially inhibit activity of a targeted enzyme or receptor.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### Exemplary Embodiments

Accordingly, provided herein are gapmer antisense oligonucleotides (ASOs), comprising a sequence that hybridizes to a target nucleic acid sequence of a long non-coding RNA (lncRNA). In some embodiments, the lncRNA regulates expression of FOXG1. In some embodiments, the lncRNA reduces expression of FOXG1 messenger RNA. In some embodiments, the lncRNA reduces transcription of FOXG1 messenger RNA molecule. In some embodiments, the lncRNA reduces expression of FOXG1 protein. In some embodiments, the lncRNA reduces translation of a FOXG1 protein molecule.

Embodiment 1. A gapmer antisense oligonucleotide comprising a sequence that hybridizes to a target nucleic acid sequence of a long non-coding RNA (lncRNA), wherein the gapmer comprises a structure having the formula: X—Y—Z wherein: X is a 5′ wing region, Y is a gap region, and Z is a 3′ wing region; and wherein X, Y, and/or Z comprise one or more modifications.

Embodiment 2. The gapmer antisense oligonucleotide of embodiment 1, wherein the lncRNA regulates expression of FOXG1.

Embodiment 3. The gapmer antisense oligonucleotide of embodiment 2, wherein the lncRNA reduces expression of FOXG1 messenger RNA.

Embodiment 4. The gapmer antisense oligonucleotide of embodiment 2, wherein the lncRNA reduces transcription of FOXG1 messenger RNA molecule.

Embodiment 5. The gapmer antisense oligonucleotide of embodiment 2, wherein the lncRNA reduces expression of FOXG1 protein.

Embodiment 6. The gapmer antisense oligonucleotide of embodiment 2, wherein the lncRNA reduces translation of a FOXG1 protein molecule.

Embodiment 7. The gapmer antisense oligonucleotide of any one of embodiments 1 to 6, wherein X comprises one or more nucleoside modifications.

Embodiment 8. The gapmer antisense oligonucleotide of any one of embodiments 1 to 7, wherein Z comprises one or more nucleoside modifications.

Embodiment 9. The gapmer antisense oligonucleotide of any one of embodiments 1 to 8, wherein X and Z each comprise one or more nucleoside modifications.

Embodiment 10. The gapmer antisense oligonucleotide of any one of embodiments 1 to 9, wherein the one or more nucleoside modifications comprises a LNA (2′-4′ biradicle bridged) nucleoside, 2′ substituted nucleoside (e.g., 2′-O-alkyl-RNA, 2′-O-methyl-RNA, 2′-alkoxy-RNA, 2′-O-methoxyethyl-oligos (MOE), 2′-amino-DNA, 2′-Fluoro-RNA, and 2′-F-ANA nucleosides), nucleobase modifications. Or a combination thereof

Embodiment 11. The gapmer antisense oligonucleotide of any one of embodiments 1 to 10, wherein X comprises one or more inter-nucleoside linker modifications.

Embodiment 12. The gapmer antisense oligonucleotide of any one of embodiments 1 to 11, wherein Y comprises one or more inter-nucleoside linker modifications.

Embodiment 13. The gapmer antisense oligonucleotide of any one of embodiments 1 to 12, wherein Z comprises one or more inter-nucleoside linker modifications.

Embodiment 14. The gapmer antisense oligonucleotide of any one of embodiments 1 to 13, wherein X and Z each comprise one or more inter-nucleoside linker modifications.

Embodiment 15. The gapmer antisense oligonucleotide of any one of embodiments 1 to 13, wherein X, Y, and Z each comprise one or more inter-nucleoside linker modifications.

Embodiment 16. The gapmer antisense oligonucleotide of any one of embodiments 1 to 15, wherein the sequence is complementary to the target nucleic acid sequence of a long non-coding RNA (lncRNA).

Embodiment 17. The gapmer antisense oligonucleotide of any one of embodiments 1 to 16, wherein the long non-coding RNA (lncRNA) is located within 1 kilobases (kb), 2 kb, 5 kb, 8 kb, or 10 kb of a gene encoding FOXG1.

Embodiment 18. The gapmer antisense oligonucleotide of any one of embodiments 1 to 17, wherein the long non-coding RNA (lncRNA) is FOXG1-AS1, long intergenic non-protein coding RNA 1551, long intergenic non-protein coding RNA 2282 (LINC02282), or a combination thereof.

Embodiment 19. The gapmer antisense oligonucleotide of any one of embodiments 1 to 18, wherein the sequence comprises a nucleobase sequence as set forth in any one of Table 3.

Embodiment 20. The gapmer antisense oligonucleotide of any one of embodiments 1 to 18, wherein the gapmer antisense oligonucleotide comprises a nucleobase sequence as set forth in any one of Table 4.

Embodiment 21. The gapmer antisense oligonucleotide of any one of embodiments 1 to 18, wherein the gapmer antisense oligonucleotide hybridizes to the target nucleic acid sequence comprising or adjacent to any one or more of the positions provided in Table 3.

Embodiment 22. The gapmer antisense oligonucleotide of embodiment 21, wherein adjacent to any one or more of the positions provided in Table 3 comprises base positions within 20, 40, 50, 75, 100, or 150 base positions 5′ and/or 3′.

Embodiment 23. The gapmer antisense oligonucleotide of any one of embodiments 1 to 22, wherein the gapmer antisense oligonucleotide hybridizes to the target nucleic acid sequence comprising or adjacent to any one or more of the positions provided in Table 4,

Embodiment 24. The gapmer antisense oligonucleotide of embodiment 23, wherein adjacent to any one or more of the positions provided in Table 4 comprises base positions within 20, 40, 50, 75, 100, or 150 base positions 5′ and/or 3′.

Embodiment 25. The gapmer antisense oligonucleotide of any one of embodiments 1 to 24, wherein hybridization of the sequence of the gapmer antisense oligonucleotide to the target nucleic acid sequence increases degradation of the lncRNA.

Embodiment 26. The gapmer antisense oligonucleotide of any one of embodiments 1 to 24, wherein hybridization of the sequence of the gapmer antisense oligonucleotide to the target nucleic acid sequence increases expression of FOXG1.

Embodiment 27. The gapmer antisense oligonucleotide of embodiment 26, wherein expression of FOXG1 is mRNA expression.

Embodiment 28. The gapmer antisense oligonucleotide of embodiment 26, wherein expression of FOXG1 is protein expression.

Embodiment 29. A composition comprising one or more of the gapmer antisense oligonucleotides of any one of embodiments 1 to 28.

Embodiment 30. A pharmaceutical composition comprising the gapmer antisense oligonucleotide of any one of embodiments 1 to 29 and a pharmaceutically acceptable carrier or diluent.

Embodiment 31. A method of modulating expression of FOXG1 in a cell, comprising contacting the cell with the gapmer antisense oligonucleotide of any one of embodiments 1 to 29.

Embodiment 32. The method of embodiment 31, wherein the cell is a located in a brain of an individual.

Embodiment 33. The method of embodiment 32, wherein the individual is a human.

Embodiment 34. The method of embodiment 32, wherein the individual comprises reduced FOXG1 expression or a FOXG1 deficiency.

Embodiment 35. The method of embodiment 32, wherein the individual has a FOXG1 disease or disorder.

Embodiment 36. The method of embodiment 35, wherein the FOXG1 disease or disorder is FOXG1 syndrome.

Embodiment 37. The method of any one of embodiments 31 to 36, wherein modulating expression comprises increasing expression of a FOXG1 protein in the cell.

Embodiment 38. The method of any one of embodiments 31 to 38, wherein modulating expression comprises increasing translation of a FOXG1 protein in the cell.

Embodiment 39. A method of treating or ameliorating a FOXG1 disease or disorder in an individual having, or at risk of having, the FOXG1 disease or disorder, comprising administering to the individual the gapmer antisense oligonucleotide of any one of embodiments 1 to 29.

Embodiment 40. The method of embodiment 39, wherein the individual is a human.

Embodiment 41. The method of embodiment 40, wherein the human is an unborn human.

Embodiment 42. The method of any one of embodiments 39 to 41, wherein the individual comprises a mutated FOXG1 gene, a reduced expression of FOXG1, a deficiency of FOXG1, or a combination thereof.

Embodiment 43. The method of any one of embodiments 39 to 42, wherein the FOXG1 disease or disorder is FOXG1 syndrome.

### EXAMPLES

The following examples are included for illustrative purposes only and are not intended to limit the scope of the present disclosure.

### Example 1: Design and Selection of ASOs

Antisense oligonucleotides (“ASOs” or “oligos”) against the human FOXG1-AS1, LINC01151, and LINC02282 mRNAs were chosen as follows. Twenty-mer (“20 mer”) nucleotide subsequences that were reverse-complementary to the lncRNA targets FOXG1-AS1 (NR_125758.1), LINC01551 (NR_026732.1 and NR_026731.1-merged exons) LINC02282 (NR_135255.1) were assembled. Thermal and sequence characteristics were then used to initially design a subset of the oligos as follows:

Different characteristics were used in the initial selection step (above). In the above, Tm=Melting temperature of hybridization; Thairpin=temperature of hairpin formation; Thomodimer=temperature of homodimer formation, as predicted by the Biopython software package. These selected 20 mers were then further selected for specificity via sequence alignment to the complete human RefSeq unspliced transcriptome (downloaded Mar. 26, 2020). Alignment was conducted using the FASTA software suite.

TABLE 1
Antisense oligonucleotides targeting IncRNA

| Targets | Sequence |
|---|---|
| NR_125758.1_60-79_as | GGTATGTTTCGTGCCCATGT |
| NR_125758.1_62-81_as | GTGGTATGTTTCGTGCCCAT |
| NR_125758.1_63-82_as | TGTGGTATGTTTCGTGCCCA |
| NR_125758.1_64-83_as | ATGTGGTATGTTTCGTGCCC |
| NR_125758.1_65-84_as | AATGTGGTATGTTTCGTGCC |
| NR_125758.1_66-85_as | AAATGTGGTATGTTTCGTGC |
| NR_125758.1_67-86_as | AAAATGTGGTATGTTTCGTG |
| NR_125758.1_68-87_as | TAAAATGTGGTATGTTTCGT |
| NR_125758.1_69-88_as | GTAAAATGTGGTATGTTTCG |
| NR_125758.1_70-89_as | CGTAAAATGTGGTATGTTTC |
| NR_125758.1_71-90_as | CCGTAAAATGTGGTATGTTT |
| NR_125758.1_72-91_as | TCCGTAAAATGTGGTATGTT |
| NR_125758.1_82-101_as | GGCTACATCCTCCGTAAAAT |
| NR_125758.1_115-134_as | TCATTTATGCTTCTCCACCT |
| NR_125758.1_116-135_as | TTCATTTATGCTTCTCCACC |
| NR_125758.1_117-136_as | TTTCATTTATGCTTCTCCAC |
| NR_125758.1_118-137_as | CTTTCATTTATGCTTCTCCA |
| NR_125758.1_119-138_as | CCTTTCATTTATGCTTCTCC |
| NR_125758.1_120-139_as | GCCTTTCATTTATGCTTCTC |
| NR_125758.1_121-140_as | TGCCTTTCATTTATGCTTCT |
| NR_125758.1_122-141_as | GTGCCTTTCATTTATGCTTC |
| NR_125758.1_123-142_as | GGTGCCTTTCATTTATGCTT |
| NR_125758.1_124-143_as | AGGTGCCTTTCATTTATGCT |
| NR_125758.1_125-144_as | AAGGTGCCTTTCATTTATGC |
| NR_125758.1_155-174_as | AATTCTCTGTGCATCTTCTA |
| NR_125758.1_156-175_as | AAATTCTCTGTGCATCTTCT |
| NR_125758.1_157-176_as | GAAATTCTCTGTGCATCTTC |
| NR_125758.1_158-177_as | AGAAATTCTCTGTGCATCTT |
| NR_125758.1_167-186_as | CACAAGGTCAGAAATTCTCT |
| NR_125758.1_168-187_as | TCACAAGGTCAGAAATTCTC |
| NR_125758.1_169-188_as | GTCACAAGGTCAGAAATTCT |
| NR_125758.1_172-191_as | AACGTCACAAGGTCAGAAAT |
| NR_125758.1_205-224_as | TGGATGCCTCTGTATGGGAT |
| NR_125758.1_206-225_as | CTGGATGCCTCTGTATGGGA |
| NR_125758.1_208-227_as | ACCTGGATGCCTCTGTATGG |
| NR_125758.1_209-228_as | TACCTGGATGCCTCTGTATG |
| NR_125758.1_210-229_as | ATACCTGGATGCCTCTGTAT |
| NR_125758.1_211-230_as | AATACCTGGATGCCTCTGTA |
| NR_125758.1_212-231_as | AAATACCTGGATGCCTCTGT |
| NR_125758.1_213-232_as | GAAATACCTGGATGCCTCTG |
| NR_125758.1_214-233_as | GGAAATACCTGGATGCCTCT |
| NR_125758.1_268-287_as | ATTATAGACGAGTTGGCTCC |
| NR_125758.1_282-301_as | GCTGTTAGGAAGATATTATA |
| NR_125758.1_283-302_as | TGCTGTTAGGAAGATATTAT |
| NR_125758.1_284-303_as | CTGCTGTTAGGAAGATATTA |
| NR_125758.1_285-304_as | TCTGCTGTTAGGAAGATATT |
| NR_125758.1_286-305_as | TTCTGCTGTTAGGAAGATAT |
| NR_125758.1_287-306_as | GTTCTGCTGTTAGGAAGATA |
| NR_125758.1_288-307_as | GGTTCTGCTGTTAGGAAGAT |
| NR_125758.1_289-308_as | AGGTTCTGCTGTTAGGAAGA |
| NR_125758.1_290-309_as | CAGGTTCTGCTGTTAGGAAG |
| NR_125758.1_291-310_as | CCAGGTTCTGCTGTTAGGAA |
| NR_125758.1_292-311_as | CCCAGGTTCTGCTGTTAGGA |
| NR_125758.1_293-312_as | ACCCAGGTTCTGCTGTTAGG |
| NR_125758.1_296-315_as | GAGACCCAGGTTCTGCTGTT |
| NR_125758.1_297-316_as | TGAGACCCAGGTTCTGCTGT |
| NR_125758.1_414-433_as | CCGTACCTGTAGTTCCAGCT |
| NR_125758.1_416-435_as | TCCCGTACCTGTAGTTCCAG |
| NR_125758.1_417-436_as | TTCCCGTACCTGTAGTTCCA |
| NR_125758.1_418-437_as | TTTCCCGTACCTGTAGTTCC |
| NR_125758.1_419-438_as | TTTTCCCGTACCTGTAGTTC |
| NR_125758.1_420-439_as | GTTTTCCCGTACCTGTAGTT |
| NR_125758.1_421-440_as | AGTTTTCCCGTACCTGTAGT |
| NR_125758.1_476-495_as | CCGAAATTATTTTGTTAAAC |
| NR_125758.1_477-496_as | GCCGAAATTATTTTGTTAAA |
| NR_125758.1_478-497_as | AGCCGAAATTATTTTGTTAA |
| NR_125758.1_479-498_as | TAGCCGAAATTATTTTGTTA |
| NR_125758.1_480-499_as | ATAGCCGAAATTATTTTGTT |
| NR_125758.1_481-500_as | GATAGCCGAAATTATTTTGT |
| NR_125758.1_483-502_as | TTGATAGCCGAAATTATTTT |
| NR_125758.1_484-503_as | TTTGATAGCCGAAATTATTT |
| NR_125758.1_485-504_as | CTTTGATAGCCGAAATTATT |
| NR_125758.1_486-505_as | TCTTTGATAGCCGAAATTAT |
| NR_125758.1_488-507_as | GATCTTTGATAGCCGAAATT |
| NR_125758.1_489-508_as | TGATCTTTGATAGCCGAAAT |
| NR_125758.1_490-509_as | TTGATCTTTGATAGCCGAAA |
| NR_125758.1_491-510_as | CTTGATCTTTGATAGCCGAA |
| NR_125758.1_492-511_as | ACTTGATCTTTGATAGCCGA |
| NR_125758.1_493-512_as | CACTTGATCTTTGATAGCCG |
| NR_125758.1_494-513_as | CCACTTGATCTTTGATAGCC |
| NR_125758.1_498-517_as | TATCCCACTTGATCTTTGAT |
| NR_125758.1_499-518_as | TTATCCCACTTGATCTTTGA |
| NR_125758.1_501-520_as | ATTTATCCCACTTGATCTTT |
| NR_125758.1_502-521_as | AATTTATCCCACTTGATCTT |
| NR_125758.1_540-559_as | CCTCTATGGTATGCAAGGAG |
| NR_125758.1_552-571_as | ACCTCGACCTCTCCTCTATG |
| NR_125758.1_553-572_as | GACCTCGACCTCTCCTCTAT |
| NR_125758.1_625-644_as | GCTAGCAGACTCACACCACA |
| NR_125758.1_630-649_as | TCACGGCTAGCAGACTCACA |
| NR_125758.1_636-655_as | TGTCTCTCACGGCTAGCAGA |
| NR_125758.1_638-657_as | TCTGTCTCTCACGGCTAGCA |
| NR_125758.1_639-658_as | ATCTGTCTCTCACGGCTAGC |
| NR_125758.1_652-671_as | CCCTTTGTAATGCATCTGTC |
| NR_125758.1_653-672_as | TCCCTTTGTAATGCATCTGT |
| NR_125758.1_654-673_as | ATCCCTTTGTAATGCATCTG |
| NR_125758.1_655-674_as | CATCCCTTTGTAATGCATCT |
| NR_125758.1_656-675_as | CCATCCCTTTGTAATGCATC |
| NR_125758.1_657-676_as | TCCATCCCTTTGTAATGCAT |
| NR_125758.1_658-677_as | ATCCATCCCTTTGTAATGCA |
| NR_125758.1_659-678_as | AATCCATCCCTTTGTAATGC |
| NR_125758.1_660-679_as | AAATCCATCCCTTTGTAATG |
| NR_125758.1_661-680_as | TAAATCCATCCCTTTGTAAT |
| NR_125758.1_662-681_as | CTAAATCCATCCCTTTGTAA |
| NR_125758.1_663-682_as | ACTAAATCCATCCCTTTGTA |
| NR_125758.1_664-683_as | CACTAAATCCATCCCTTTGT |
| NR_125758.1_665-684_as | GCACTAAATCCATCCCTTTG |
| NR_125758.1_666-685_as | TGCACTAAATCCATCCCTTT |
| NR_125758.1_667-686_as | GTGCACTAAATCCATCCCTT |
| NR_125758.1_668-687_as | AGTGCACTAAATCCATCCCT |
| NR_125758.1_719-738_as | GTTTTGTTTCATTGTTCACT |
| NR_125758.1_720-739_as | AGTTTTGTTTCATTGTTCAC |
| NR_125758.1_721-740_as | AAGTTTTGTTTCATTGTTCA |
| NR_125758.1_730-749_as | CTTGGGAAGAAGTTTTGTTT |
| NR_125758.1_731-750_as | GCTTGGGAAGAAGTTTTGTT |
| NR_125758.1_764-783_as | ATCTCTTCAAACTATGGCAC |
| NR_125758.1_765-784_as | CATCTCTTCAAACTATGGCA |
| NR_125758.1_768-787_as | TGCCATCTCTTCAAACTATG |
| NR_125758.1_769-788_as | ATGCCATCTCTTCAAACTAT |
| NR_125758.1_770-789_as | GATGCCATCTCTTCAAACTA |
| NR_125758.1_863-882_as | TTGTATAAACTGTTGTTGCA |
| NR_026732.1_NR_026731.1_ | GAAGCTGAAGTGGTGTTGGG |
| merge_75-94_as | |
| NR_026732.1_NR_026731.1_ | AGAAGCTGAAGTGGTGTTGG |
| merge_76-95_as | |
| NR_026732.1_NR_026731.1_ | CTTTTCCTCGGCATCCTTCG |
| merge_171-190_as | |
| NR_026732.1_NR_026731.1_ | CCTTTTCCTCGGCATCCTTC |
| merge_172-191_as | |
| NR_026732.1_NR_026731.1_ | TCCTTTTCCTCGGCATCCTT |
| merge_173-192_as | |
| NR_026732.1_NR_026731.1_ | ATCCTTTTCCTCGGCATCCT |
| merge_174-193_as | |
| NR_026732.1_NR_026731.1_ | TATCCTTTTCCTCGGCATCC |
| merge_175-194_as | |
| NR_026732.1_NR_026731.1_ | ATATCCTTTTCCTCGGCATC |
| merge_176-195_as | |
| NR_026732.1_NR_026731.1_ | GATATCCTTTTCCTCGGCAT |
| merge_177-196_as | |
| NR_026732.1_NR_026731.1_ | TGATATCCTTTTCCTCGGCA |
| merge_178-197_as | |
| NR_026732.1_NR_026731.1_ | CCGATGCTCTGGAATCTCAA |
| merge_428-447_as | |
| NR_026732.1_NR_026731.1_ | TCCGATGCTCTGGAATCTCA |
| merge_429-448_as | |
| NR_026732.1_NR_026731.1_ | CATCCGATGCTCTGGAATCT |
| merge_431-450_as | |
| NR_026732.1_NR_026731.1_ | TCATCCGATGCTCTGGAATC |
| merge_432-451_as | |
| NR_026732.1_NR_026731.1_ | TTCATCCGATGCTCTGGAAT |
| merge_433-452_as | |
| NR_026732.1_NR_026731.1_ | ACTACCCCTATGCACGTGAG |
| merge_521-540_as | |
| NR_026732.1_NR_026731.1_ | GTTCTTCCCCAAATGCCTTT |
| merge_962-981_as | |
| NR_026732.1_NR_026731.1_ | TGTTCTTCCCCAAATGCCTT |
| merge_963-982_as | |
| NR_026732.1_NR_026731.1_ | TTGTTCTTCCCCAAATGCCT |
| merge_964-983_as | |
| NR_026732.1_NR_026731.1_ | GTTGTTCTTCCCCAAATGCC |
| merge_965-984_as | |
| NR_026732.1_NR_026731.1_ | CGTTGTTCTTCCCCAAATGC |
| merge_966-985_as | |
| NR_026732.1_NR_026731.1_ | CTTTCTCTGGAGACACATCA |
| merge_1002-1021_as | |
| NR_026732.1_NR_026731.1_ | ACTTTCTCTGGAGACACATC |
| merge_1003-1022_as | |
| NR_026732.1_NR_026731.1_ | GTTGTTTGTTTGTTTGTTTT |
| merge_1039-1058_as | |
| NR_026732.1_NR_026731.1_ | TGTTGTTTGTTTGTTTGTTT |
| merge_1040-1059_as | |
| NR_026732.1_NR_026731.1_ | TTGTTGTTTGTTTGTTTGTT |
| merge_1041-1060_as | |
| NR_026732.1_NR_026731.1_ | GTTGTTGTTTGTTTGTTTGT |
| merge_1042-1061_as | |
| NR_026732.1_NR_026731.1_ | TGTTGTTGTTTGTTTGTTTG |
| merge_1043-1062_as | |
| NR_026732.1_NR_026731.1_ | TTGTTGTTGTTTGTTTGTTT |
| merge_1044-1063_as | |
| NR_026732.1_NR_026731.1_ | ATTGTTGTTGTTTGTTTGTT |
| merge_1045-1064_as | |
| NR_026732.1_NR_026731.1_ | TATTGTTGTTGTTTGTTTGT |
| merge_1046-1065_as | |
| NR_026732.1_NR_026731.1_ | TTATTGTTGTTGTTTGTTTG |
| merge_1047-1066_as | |
| NR_026732.1_NR_026731.1_ | GTTTATTGTTGTTGTTTGTT |
| merge_1049-1068_as | |
| NR_026732.1_NR_026731.1_ | TGTTTATTGTTGTTGTTTGT |
| merge_1050-1069_as | |
| NR_026732.1_NR_026731.1_ | TTGTTTATTGTTGTTGTTTG |
| merge_1051-1070_as | |
| NR_026732.1_NR_026731.1_ | GTTGTTTATTGTTGTTGTTT |
| merge_1052-1071_as | |
| NR_026732.1_NR_026731.1_ | AGTTGTTTATTGTTGTTGTT |
| merge_1053-1072_as | |
| NR_026732.1_NR_026731.1_ | AAGTTGTTTATTGTTGTTGT |
| merge_1054-1073_as | |
| NR_026732.1_NR_026731.1_ | AGTGGAATGAGTCAGCCCGA |
| merge 1548-1567_as | |
| NR_026732.1_NR_026731.1_ | AAGTGGAATGAGTCAGCCCG |
| merge_1549-1568_as | |
| NR_026732.1_NR_026731.1_ | AAAGTGGAATGAGTCAGCCC |
| merge_1550-1569_as | |
| NR_026732.1_NR_026731.1_ | CCTGCTGGATAGGAATTAAT |
| merge_2247-2266_as | |
| NR_026732.1_NR_026731.1_ | GCCTGCTGGATAGGAATTAA |
| merge_2248-2267_as | |
| NR_026732.1_NR_026731.1_ | TTAAAGCCTGCTGGATAGGA |
| merge_2253-2272_as | |
| NR_026732.1_NR_026731.1_ | TGTTAAAGCCTGCTGGATAG |
| merge_2255-2274_as | |
| NR_026732.1_NR_026731.1_ | TTGTTAAAGCCTGCTGGATA |
| merge_2256-2275_as | |
| NR_026732.1_NR_026731.1_ | TTTGTTAAAGCCTGCTGGAT |
| merge_2257-2276_as | |
| NR_026732.1_NR_026731.1_ | TTTTGTTAAAGCCTGCTGGA |
| merge_2258-2277_as | |
| NR_026732.1_NR_026731.1_ | TTTTTGTTAAAGCCTGCTGG |
| merge_2259-2278_as | |
| NR_026732.1_NR_026731.1_ | GTTTTTGTTAAAGCCTGCTG |
| merge_2260-2279_as | |
| NR_026732.1_NR_026731.1_ | AGTTTTTGTTAAAGCCTGCT |
| merge_2261-2280_as | |
| NR_026732.1_NR_026731.1_ | TAGTTTTTGTTAAAGCCTGC |
| merge_2262-2281_as | |
| NR_026732.1_NR_026731.1_ | TCTTTAGTAGCTTTCATGGC |
| merge_2319-2338_as | |
| NR_026732.1_NR_026731.1_ | CTGGCTTTTCTTTAGTAGCT |
| merge_2327-2346_as | |
| NR_026732.1_NR_026731.1_ | GCTGTTTCTGGCTTTTCTTT |
| merge_2334-2353_as | |
| NR_026732.1_NR_026731.1_ | CGCTGTTTCTGGCTTTTCTT |
| merge_2335-2354_as | |
| NR_026732.1_NR_026731.1_ | ACGCTGTTTCTGGCTTTTCT |
| merge_2336-2355_as | |
| NR_026732.1_NR_026731.1_ | TACGCTGTTTCTGGCTTTTC |
| merge_2337-2356_as | |
| NR_026732.1_NR_026731.1_ | TTACGCTGTTTCTGGCTTTT |
| merge_2338-2357_as | |
| NR_026732.1_NR_026731.1_ | CTTACGCTGTTTCTGGCTTT |
| merge_2339-2358_as | |
| NR_026732.1_NR_026731.1_ | TCTTACGCTGTTTCTGGCTT |
| merge_2340-2359_as | |
| NR_026732.1_NR_026731.1_ | TTCTTACGCTGTTTCTGGCT |
| merge_2341-2360_as | |
| NR_026732.1_NR_026731.1_ | ATTCTTACGCTGTTTCTGGC |
| merge_2342-2361_as | |
| NR_026732.1_NR_026731.1_ | CCTCGTCTCTGAATCATATT |
| merge_2372-2391_as | |
| NR_026732.1_NR_026731.1_ | CACAATAGTAGTGGCCTTGT |
| merge_2428-2447_as | |
| NR_026732.1_NR_026731.1_ | TCACAATAGTAGTGGCCTTG |
| merge_2429-2448_as | |
| NR_026732.1_NR_026731.1_ | TTCACAATAGTAGTGGCCTT |
| merge_2430-2449_as | |
| NR_026732.1_NR_026731.1_ | ATTCACAATAGTAGTGGCCT |
| merge_2431-2450_as | |
| NR_026732.1_NR_026731.1_ | CCATGTTGACTTAGTTGGTC |
| merge_2675-2694_as | |
| NR_026732.1_NR_026731.1_ | ACCATGTTGACTTAGTTGGT |
| merge_2676-2695_as | |
| NR_026732.1_NR_026731.1_ | GCTACCATGTCTGACTAATT |
| merge_2731-2750_as | |
| NR_026732.1_NR_026731.1_ | TGCTACCATGTCTGACTAAT |
| merge_2732-2751_as | |
| NR_026732.1_NR_026731.1_ | GTGCTACCATGTCTGACTAA |
| merge_2733-2752_as | |
| NR_026732.1_NR_026731.1_ | TGTGCTACCATGTCTGACTA |
| merge_2734-2753_as | |
| NR_026732.1_NR_026731.1_ | ATGTGCTACCATGTCTGACT |
| merge_2735-2754_as | |
| NR_026732.1_NR_026731.1_ | CATGTGCTACCATGTCTGAC |
| merge_2736-2755_as | |
| NR_026732.1_NR_026731.1_ | TGGGTGATATTTGGTTCCAA |
| merge_3554-3573_as | |
| NR_026732.1_NR_026731.1_ | CTGAGGAAATTGATGGTATA |
| merge_3673-3692_as | |
| NR_026732.1_NR_026731.1_ | ACTGAGGAAATTGATGGTAT |
| merge_3674-3693_as | |
| NR_026732.1_NR_026731.1_ | GACCGTACGAGGGAATTTTA |
| merge_3710-3729_as | |
| NR_026732.1_NR_026731.1_ | TGACCGTACGAGGGAATTTT |
| merge_3711-3730_as | |
| NR_026732.1_NR_026731.1_ | TTGACCGTACGAGGGAATTT |
| merge_3712-3731_as | |
| NR_026732.1_NR_026731.1_ | TTTGACCGTACGAGGGAATT |
| merge_3713-3732_as | |
| NR_026732.1_NR_026731.1_ | TTTTGACCGTACGAGGGAAT |
| merge_3714-3733_as | |
| NR_135255.1_89-108_as | GCCTTCTGTACTGTGATGGG |
| NR_135255.1_90-109_as | AGCCTTCTGTACTGTGATGG |
| NR_135255.1_91-110_as | AAGCCTTCTGTACTGTGATG |
| NR_135255.1_192-211_as | ATGTGTGGGATGTAGGTAGG |
| NR_135255.1_193-212_as | AATGTGTGGGATGTAGGTAG |
| NR_135255.1_194-213_as | AAATGTGTGGGATGTAGGTA |
| NR_135255.1_204-223_as | GGGACTCCTGAAATGTGTGG |
| NR_135255.1_230-249_as | CGTTCTGTGTTTTGTAGAAT |
| NR_135255.1_232-251_as | GTCGTTCTGTGTTTTGTAGA |
| NR_135255.1_233-252_as | GGTCGTTCTGTGTTTTGTAG |
| NR_135255.1_234-253_as | TGGTCGTTCTGTGTTTTGTA |
| NR_135255.1_235-254_as | ATGGTCGTTCTGTGTTTTGT |
| NR_135255.1_236-255_as | TATGGTCGTTCTGTGTTTTG |
| NR_135255.1_237-256_as | ATATGGTCGTTCTGTGTTTT |
| NR_135255.1_243-262_as | TGGCTCATATGGTCGTTCTG |
| NR_135255.1_244-263_as | GTGGCTCATATGGTCGTTCT |
| NR_135255.1_245-264_as | AGTGGCTCATATGGTCGTTC |
| NR_135255.1_246-265_as | AAGTGGCTCATATGGTCGTT |
| NR_135255.1_455-474_as | CTCAGTGACAGCTAGGTGGA |
| NR_135255.1_456-475_as | TCTCAGTGACAGCTAGGTGG |
| NR_135255.1_458-477_as | ATTCTCAGTGACAGCTAGGT |
| NR_135255.1_462-481_as | CCGAATTCTCAGTGACAGCT |
| NR_135255.1_586-605_as | CAATGCAGAGTTTCTATTAC |
| NR_135255.1_669-688_as | CCCATTCCCAGGATGTTAGA |
| NR_135255.1_670-689_as | TCCCATTCCCAGGATGTTAG |
| NR_135255.1_671-690_as | TTCCCATTCCCAGGATGTTA |
| NR_135255.1_672-691_as | CTTCCCATTCCCAGGATGTT |
| NR_135255.1_673-692_as | ACTTCCCATTCCCAGGATGT |
| NR_135255.1_674-693_as | TACTTCCCATTCCCAGGATG |
| NR_135255.1_675-694_as | TTACTTCCCATTCCCAGGAT |
| NR_135255.1_676-695_as | GTTACTTCCCATTCCCAGGA |
| NR_135255.1_677-696_as | TGTTACTTCCCATTCCCAGG |
| NR_135255.1_678-697_as | GTGTTACTTCCCATTCCCAG |
| NR_135255.1_679-698_as | AGTGTTACTTCCCATTCCCA |
| NR_135255.1_680-699_as | CAGTGTTACTTCCCATTCCC |
| NR_135255.1_690-709_as | CCACCGATCCCAGTGTTACT |
| NR_135255.1_763-782_as | TTTCCATTCCTCTCTTCCAT |
| NR_135255.1_764-783_as | CTTTCCATTCCTCTCTTCCA |
| NR_135255.1_766-785_as | GCCTTTCCATTCCTCTCTTC |
| NR_135255.1_767-786_as | TGCCTTTCCATTCCTCTCTT |
| NR_135255.1_768-787_as | TTGCCTTTCCATTCCTCTCT |
| NR_135255.1_769-788_as | TTTGCCTTTCCATTCCTCTC |
| NR_135255.1_770-789_as | TTTTGCCTTTCCATTCCTCT |
| NR_135255.1_771-790_as | CTTTTGCCTTTCCATTCCTC |
| NR_135255.1_772-791_as | TCTTTTGCCTTTCCATTCCT |
| NR_135255.1_773-792_as | TTCTTTTGCCTTTCCATTCC |
| NR_135255.1_833-852_as | TGCTGATGGTGGGACTTTTT |
| NR_135255.1_834-853_as | TTGCTGATGGTGGGACTTTT |
| NR_135255.1_835-854_as | TTTGCTGATGGTGGGACTTT |
| NR_135255.1_836-855_as | TTTTGCTGATGGTGGGACTT |
| NR_135255.1_837-856_as | CTTTTGCTGATGGTGGGACT |
| NR_135255.1_838-857_as | TCTTTTGCTGATGGTGGGAC |
| NR_135255.1_839-858_as | TTCTTTTGCTGATGGTGGGA |
| NR_135255.1_840-859_as | CTTCTTTTGCTGATGGTGGG |
| NR_135255.1_841-860_as | ACTTCTTTTGCTGATGGTGG |
| NR_135255.1_842-861_as | GACTTCTTTTGCTGATGGTG |
| NR_135255.1_843-862_as | AGACTTCTTTTGCTGATGGT |
| NR_135255.1_844-863_as | GAGACTTCTTTTGCTGATGG |
| NR_135255.1_845-864_as | AGAGACTTCTTTTGCTGATG |
| NR_135255.1_862-881_as | GCTGCTATTTTAGAGGAAGA |
| NR_135255.1_863-882_as | GGCTGCTATTTTAGAGGAAG |
| NR_135255.1_867-886_as | CTTTGGCTGCTATTTTAGAG |
| NR_135255.1_869-888_as | CTCTTTGGCTGCTATTTTAG |
| NR_135255.1_870-889_as | TCTCTTTGGCTGCTATTTTA |
| NR_135255.1_876-895_as | ATTTTCTCTCTTTGGCTGCT |
| NR_135255.1_978-997_as | GTTCAGAAATTGGGATTAAT |
| NR_135255.1_979-998_as | TGTTCAGAAATTGGGATTAA |
| NR_135255.1_981-1000_as | GCTGTTCAGAAATTGGGATT |
| NR_135255.1_982-1001_as | TGCTGTTCAGAAATTGGGAT |
| NR_135255.1_983-1002_as | ATGCTGTTCAGAAATTGGGA |
| NR_135255.1_984-1003_as | AATGCTGTTCAGAAATTGGG |
| NR_135255.1_993-1012_as | GCTAAGTAAAATGCTGTTCA |
| NR_135255.1_994-1013_as | TGCTAAGTAAAATGCTGTTC |
| NR_135255.1_1226-1245_as | TTTCCAACAGGCTCTCGTTT |
| NR_135255.1_1227-1246_as | CTTTCCAACAGGCTCTCGTT |
| NR_135255.1_1228-1247_as | CCTTTCCAACAGGCTCTCGT |
| NR_135255.1_1229-1248_as | TCCTTTCCAACAGGCTCTCG |
| NR_135255.1_1327-1346_as | GGTAGAATGGGAAAGGTTTT |
| NR_135255.1_1328-1347_as | GGGTAGAATGGGAAAGGTTT |
| NR_135255.1_1329-1348_as | TGGGTAGAATGGGAAAGGTT |
| NR_135255.1_1330-1349_as | CTGGGTAGAATGGGAAAGGT |
| NR_135255.1_1536-1555_as | GCACAAGTGGCAAAGCAAAA |
| NR_135255.1_1537-1556_as | TGCACAAGTGGCAAAGCAAA |
| NR_135255.1_1545-1564_as | AGATCTGTTGCACAAGTGGC |

### Example 2: Identification of ASOs that Increase FOXG1 Expression in a Cell

The MOE gapmer antisense oligonucleotides (ASOs) designed and selected in Example 1 were tested for the ability to increase FOXG1 expression in cells. In order to screen gapmer antisense oligonucleotides (ASOs), CCF-STTG1 cells were obtained from ATCC (ATCC in partnership with LGC Standards, Wesel, Germany, cat. #ATCC-CRL-1718), cultured in RPMI-1540 (#30-2001, ATCC in partnership with LGC Standards, Wesel, Germany), supplemented to contain 10% fetal calf serum (1248D, Biochrom GmbH, Berlin, Germany), and 100 U/ml Penicillin/100 μg/ml Streptomycin (A2213, Biochrom GmbH, Berlin, Germany). Cells were grown at 37° C. in an atmosphere with 5% CO₂ in a humidified incubator. For ASO transfection, CCF-STTG1 cells were seeded at a density of 15,000 cells/well into 96-well tissue culture plates (#655180, GBO, Germany).

In CCF-STTG1 cells, transfection of ASOs was carried out with Lipofectamine2000 (Invitrogen/Life Technologies, Karlsruhe, Germany) according to manufacturer's instructions for reverse transfection with 0.5 μL Lipofectamine2000 per well. The single dose screen was performed with ASOs in quadruplicates at 50 nM, with an ASO targeting AHSA1 (MOE-gapmer) and mock transfected cells as controls. ASOs were targeting one out of three lncRNAs expected to influence expression levels of FoxG1, so that FoxG1 mRNA expression was the readout. After 24 h of incubation (48 h incubation time resulted in high toxicity, visible in the rounding up of cells and low GapDH levels and was therefore neglected for analysis) with ASOs, medium was removed and cells were lysed in 150 μl Medium-Lysis Mixture (1 volume lysis mixture, 2 volumes cell culture medium) and then incubated at 53° C. for 30 minutes. Quantigene-Singleplex assay was performed according to manufacturer's instructions (ThermoFisher, Germany) with probesets to human FoxG1 and to GapDH for normalization. Luminescence was read using 1420 Luminescence Counter (WALLAC VICTOR Light, Perkin Elmer, Rodgau-Jugesheim, Germany) following 30 minutes incubation at RT in the dark.

In a subsequent experiment, 21 ASOs from the single dose screen were selected, which either produced promising results with regards to FoxG1 upregulation, or served as controls which had down-regulated FoxG1 in the initial screen. ASOs were transfected in three concentrations, namely 50 nM, 20 nM, and 2 nM, whereas Ahsa1 at 50 nM and 2 nM and mock transfected cells served as controls.

The Ahsa1-ASO (one 2′-OMe and one MOE-modified) served at the same time as unspecific control for respective target mRNA expression and as a positive control to analyze transfection efficiency with regards to Ahsa1 mRNA level. By hybridization with an Ahsa1 probeset, the mock transfected wells served as controls for Ahsa1 mRNA level. Transfection efficiency for each 96-well plate and both doses in the dual dose screen were calculated by relating Ahsa1-level with Ahsa1-ASO (normalized to GapDH) to Ahsa1-level obtained with mock controls.

For each well, the target mRNA level was normalized to the respective GAPDH mRNA level. The activity of a given ASO was expressed as percent mRNA concentration of the respective target (normalized to GAPDH mRNA) in treated cells, relative to the target mRNA concentration (normalized to GAPDH mRNA) averaged across control wells (set as 100% target expression). mRNA expression was quantified using QuantiGene. Table 2 provides the Human FoxG1 QG2.0 probeset (Accession #NM_005249) and Human GapDH QG2.0 probeset (Accession #NM_002046). Oligosequences “CEs” and “LEs” are depicted without the proprietary parts of their sequences. Cross reactivity with the cynomolgus sequence was obtained by adding additional probes.

TABLE 2
QuantiGene Probesets.

| Oligo name | Sequence 5′-3′ | Accession#, position & function |
|---|---|---|
| QG2_hsFoxG1_1 | ggccagcttggcccg | NM_005249.1334.1348.LE |
| QG2_hsFoxG1_2 | gcgcaccgcgcttgaa | NM_005249.1349.1364.LE |
| QG2_hsFoxG1_3 | gccggtggaggtgaggc | NM_005249.1365.1381.CE |
| QG2_hsFoxG1_4 | cgcggtccatgaaggtgag | NM_005249.1382.1400.LE |
| QG2_hsFoxG1_5 | gccagtagagggagccgg | NM_005249.1401.1418.LE |
| QG2_hsFoxG1_6 | gacaggaagggcgacatgg | NM_005249.1419.1437.BL |
| QG2_hsFoxG1_7 | gcgggggtggtgcagg | NM_005249.1438.1453.BL |
| QG2_hsFoxG1_8 | tgtaactcaaagtgctgctggc | NM_005249.1454.1475.CE |
| QG2_hsFoxG1_9 | gccgacgtggtgccgt | NM_005249.1476.1491.LE |
| QG2_hsFoxG1_10 | atggggtggctggggtag | NM_005249.1492.1509.LE |
| QG2_hsFoxG1_11 | tcaacacggagctgtagggc | NM_005249.1510.1529.CE |
| QG2_hsFoxG1_12 | gttgcccagcgagttctgag | NM_005249.1530.1549.LE |
| QG2_hsFoxG1_13 | gcggtggagaaggagtggtt | NM_005249.1550.1569.LE |
| QG2_hsFoxG1_14 | ccacgctcaggccgttg | NM_005249.1570.1586.BL |
| QG2_hsFoxG1_15 | cccgttgaccagccggt | NM 005249.1587.1603.CE |
| QG2_hsFoxG1_16 | cgtggcgtacgggatctc | NM_005249.1604.1621.LE |
| QG2_hsFoxG1_17 | gcggccgtgaggtggtg | NM_005249.1622.1638.LE |
| QG2_hsFoxG1_18 | gaggcggctagcgcg | NM_005249.1639.1653.CE |
| QG2_hsFoxG1_19 | caggccgcagggcacc | NM_005249.1654.1669.LE |
| QG2_hsFoxG1_20 | ccagagcagggcaccga | NM_005249.1670.1686.LE |
| QG2_hsFoxG1_21 | caggggttgagggagtaggtc | NM_005249.1687.1707.CE |
| QG2_hsFoxG1_22 | gcgagcaggttgacggag | NM_005249.1708.1725.LE |
| QG2_hsFoxG1_23 | gaaaaagtaactggtctggccc | NM_005249.1726.1747.LE |
| QG2_hsFoxG1_24 | ggtgcgggacgtgggg | NM_005249.1748.1763.CE |
| QG2_hsFoxG1_25 | tgctctgcgaagtcattgacg | NM_005249.1764.1784.LE |
| QG2_hsFoxG1_26 | ggcgctcatggacgtgc | NM_005249.1785.1801.LE |
| QG2_hsFoxG1_27 | aggaggacgcggccct | NM_005249.1802.1817.CE |

Tables 3 and 4 shows gapmer antisense oligonucleotides (ASOs) that increase FOXG1 expression, providing the lncRNA target, OligoID, and position.

TABLE 3
Antisense oligonucleotides (ASOs) increasing FOXG1 expression

| | | | LSM |
|---|---|---|---|
| Target | OligoID | Position | (% FOXG1) |
| FOXG1-AS1 | NR_125758.1_68-87_as | 68 | 119.8358 |
| FOXG1-AS1 | NR_125758.1_69-88_as | 69 | 105.2692 |
| FOXG1-AS1 | NR_125758.1_71-90_as | 71 | 129.8516 |
| FOXG1-AS1 | NR_125758.1_72-91_as | 72 | 119.8393 |
| FOXG1-AS1 | NR_125758.1_115-134_as | 115 | 115.2154 |
| FOXG1-AS1 | NR_125758.1_116-135_as | 116 | 120.8962 |
| FOXG1-AS1 | NR_125758.1_117-136_as | 117 | 125.1282 |
| FOXG1-AS1 | NR_125758.1_118-137_as | 118 | 134.2027 |
| FOXG1-AS1 | NR_125758.1_119-138_as | 119 | 145.4411 |
| FOXG1-AS1 | NR 125758.1_120-139_as | 120 | 114.0783 |
| FOXG1-AS1 | NR_125758.1_121-140_as | 121 | 133.4272 |
| FOXG1-AS1 | NR_125758.1_205-224_as | 205 | 121.4066 |
| FOXG1-AS1 | NR_125758.1_206-225_as | 206 | 178.3451 |
| FOXG1-AS1 | NR_125758.1_208-227_as | 208 | 168.5946 |
| FOXG1-AS1 | NR_125758.1_209-228_as | 209 | 168.4846 |
| FOXG1-AS1 | NR_125758.1_210-229_as | 210 | 142.1497 |
| FOXG1-AS1 | NR_125758.1_290-309_as | 290 | 116.9382 |
| FOXG1-AS1 | NR_125758.1_291-310_as | 291 | 110.7735 |
| FOXG1-AS1 | NR_125758.1_292-311_as | 292 | 139.5714 |
| FOXG1-AS1 | NR 125758.1_293-312_as | 293 | 131.8477 |
| FOXG1-AS1 | NR_125758.1_296-315_as | 296 | 148.1122 |
| FOXG1-AS1 | NR_125758.1_297-316_as | 297 | 163.8187 |
| FOXG1-AS1 | NR_125758.1_414-433_as | 414 | 142.654 |
| FOXG1-AS1 | NR_125758.1_416-435_as | 416 | 166.4228 |
| FOXG1-AS1 | NR_125758.1_417-436_as | 417 | 139.3282 |
| FOXG1-AS1 | NR_125758.1_418-437_as | 418 | 115.4389 |
| FOXG1-AS1 | NR_125758.1_476-495_as | 476 | 116.6715 |
| FOXG1-AS1 | NR_125758.1_478-497_as | 478 | 113.534 |
| FOXG1-AS1 | NR_125758.1_479-498_as | 479 | 139.7402 |
| FOXG1-AS1 | NR_125758.1_480-499_as | 480 | 118.2394 |
| FOXG1-AS1 | NR_125758.1_483-502_as | 483 | 111.2582 |
| FOXG1-AS1 | NR_125758.1_489-508_as | 489 | 106.8954 |
| FOXG1-AS1 | NR_125758.1_490-509_as | 490 | 113.217 |
| FOXG1-AS1 | NR_125758.1_494-513_as | 494 | 137.5701 |
| FOXG1-AS1 | NR_125758.1_498-517_as | 498 | 114.7837 |
| FOXG1-AS1 | NR_125758.1_499-518_as | 499 | 100.4794 |
| FOXG1-AS1 | NR_125758.1_540-559_as | 540 | 115.324 |
| FOXG1-AS1 | NR_125758.1_636-655_as | 636 | 107.0681 |
| FOXG1-AS1 | NR_125758.1_653-672_as | 653 | 109.7666 |
| FOXG1-AS1 | NR_125758.1_654-673_as | 654 | 102.4918 |
| FOXG1-AS1 | NR_125758.1_655-674_as | 655 | 118.0954 |
| FOXG1-AS1 | NR_125758.1_656-675_as | 656 | 106.2447 |
| FOXG1-AS1 | NR 125758.1_657-676_as | 657 | 102.3042 |
| FOXG1-AS1 | NR_125758.1_658-677_as | 658 | 117.0233 |
| FOXG1-AS1 | NR_125758.1_660-679_as | 660 | 142.5884 |
| FOXG1-AS1 | NR_125758.1_661-680_as | 661 | 146.0999 |
| FOXG1-AS1 | NR 125758.1_662-681_as | 662 | 133.1997 |
| FOXG1-AS1 | NR_125758.1_663-682_as | 663 | 142.6474 |
| FOXG1-AS1 | NR_125758.1_664-683_as | 664 | 130.6147 |
| FOXG1-AS1 | NR_125758.1_665-684_as | 665 | 119.5423 |
| FOXG1-AS1 | NR_125758.1_666-685_as | 666 | 144.1696 |
| FOXG1-AS1 | NR_125758.1_667-686_as | 667 | 121.2138 |
| FOXG1-AS1 | NR 125758.1_720-739_as | 720 | 111.9995 |
| FOXG1-AS1 | NR_125758.1_721-740_as | 721 | 124.5812 |
| FOXG1-AS1 | NR_125758.1_730-749_as | 730 | 126.9396 |
| FOXG1-AS1 | NR_125758.1_731-750_as | 731 | 121.3403 |
| FOXG1-AS1 | NR_125758.1_764-783_as | 764 | 128.0099 |
| FOXG1-AS1 | NR_125758.1_765-784_as | 765 | 145.519 |
| FOXG1-AS1 | NR_125758.1_768-787_as | 768 | 146.7117 |
| FOXG1-AS1 | NR_125758.1_769-788_as | 769 | 137.4396 |
| FOXG1-AS1 | NR_125758.1_770-789_as | 770 | 124.9839 |
| LINC01551 | NR_026732.1_NR_026731 . . . 1_ | 171 | 101.2436 |
| | merge_171-190_as | | |
| LINC01551 | NR_026732.1_NR_026731 . . . 1_ | 172 | 103.6003 |
| | merge 172-191_as | | |
| LINC01551 | NR_026732.1_NR_026731 . . . 1_ | 431 | 118.3662 |
| | merge_431-450_as | | |
| LINC01551 | NR_026732.1_NR_026731 . . . 1_ | 432 | 117.9914 |
| | merge_432-451_as | | |
| LINC01551 | NR_026732.1_NR_026731 . . . 1_ | 433 | 125.6478 |
| | merge_433-452_as | | |
| LINC01551 | NR_026732.1_NR_026731 . . . 1_ | 521 | 135.4429 |
| | merge_521-540_as | | |
| LINC01551 | NR_026732.1_NR_026731 . . . 1_ | 962 | 144.108 |
| | merge_962-981_as | | |
| LINC01551 | NR_026732.1_NR_026731 . . . 1_ | 963 | 159.1389 |
| | merge_963-982_as | | |
| LINC01551 | NR_026732.1_NR_026731 . . . 1_ | 964 | 192.7082 |
| | merge_964-983_as | | |
| LINC01551 | NR_026732.1_NR_026731 . . . 1_ | 965 | 129.34 |
| | merge_965-984_as | | |
| LINC01551 | NR_026732.1_NR_026731 . . . 1_ | 966 | 131.5178 |
| | merge_966-985_as | | |
| LINC01551 | NR_026732.1_NR_026731 . . . 1_ | 1003 | 111.0948 |
| | merge_1003-1022_as | | |
| LINC01551 | NR_026732.1_NR_026731 . . . 1_ | 1039 | 109.8659 |
| | merge_1039-1058_as | | |
| LINC01551 | NR_026732.1_NR_026731 . . . 1_ | 1040 | 111.6455 |
| | merge_1040-1059_as | | |
| LINC01551 | NR_026732.1_NR_026731 . . . 1_ | 1041 | 135.8098 |
| | merge_1041-1060_as | | |
| LINC01551 | NR_026732.1_NR_026731 . . . 1_ | 1042 | 124.2946 |
| | merge_1042-1061_as | | |
| LINC01551 | NR_026732.1_NR_026731 . . . 1_ | 1043 | 139.7436 |
| | merge_1043-1062_as | | |
| LINC01551 | NR_026732.1_NR_026731 . . . 1_ | 1046 | 162.4164 |
| | merge_1046-1065_as | | |
| LINC01551 | NR_026732.1_NR_026731 . . . 1_ | 1047 | 193.177 |
| | merge_1047-1066_as | | |
| LINC01551 | NR_026732.1_NR_026731 . . . 1_ | 1049 | 176.6551 |
| | merge_1049-1068_as | | |
| LINC01551 | NR_026732.1_NR_026731 . . . 1_ | 1050 | 186.5224 |
| | merge_1050-1069_as | | |
| LINC01551 | NR_026732.1_NR_026731 . . . 1_ | 1051 | 211.9932 |
| | merge_1051-1070_as | | |
| LINC01551 | NR_026732.1_NR_026731 . . . 1_ | 1052 | 190.2748 |
| | merge 1052-1071_as | | |
| LINC01551 | NR_026732.1_NR_026731 . . . 1_ | 1053 | 187.2997 |
| | merge_1053-1072_as | | |
| LINC01551 | NR_026732.1_NR_026731 . . . 1_ | 1054 | 184.0324 |
| | merge_1054-1073_as | | |
| LINC01551 | NR_026732.1_NR_026731 . . . 1_ | 1548 | 154.7311 |
| | merge_1548-1567_as | | |
| LINC01551 | NR_026732.1_NR_026731 . . . 1_ | 1549 | 149.9802 |
| | merge_1549-1568_as | | |
| LINC01551 | NR_026732.1_NR_026731 . . . 1_ | 1550 | 249.4493 |
| | merge_1550-1569_as | | |
| LINC01551 | NR_026732.1_NR_026731 . . . 1_ | 2247 | 314.6698 |
| | merge_2247-2266_as | | |
| LINC01551 | NR_026732.1_NR_026731 . . . 1_ | 2248 | 200.632 |
| | merge_2248-2267_as | | |
| LINC01551 | NR_026732.1_NR_026731 . . . 1_ | 2253 | 212.562 |
| | merge_2253-2272_as | | |
| LINC01551 | NR_026732.1_NR_026731 . . . 1_ | 2255 | 140.2191 |
| | merge_2255-2274_as | | |
| LINC01551 | NR_026732.1_NR_026731 . . . 1_ | 2256 | 143.2591 |
| | merge_2256-2275_as | | |
| LINC01551 | NR_026732.1_NR_026731 . . . 1_ | 2257 | 119.5281 |
| | merge_2257-2276_as | | |
| LINC01551 | NR_026732.1_NR_026731 . . . 1_ | 2258 | 149.5404 |
| | merge_2258-2277_as | | |
| LINC01551 | NR_026732.1_NR_026731 . . . 1_ | 2259 | 108.7718 |
| | merge_2259-2278_as | | |
| LINC01551 | NR_026732.1_NR_026731 . . . 1_ | 2260 | 107.0709 |
| | merge_2260-2279_as | | |
| LINC01551 | NR_026732.1_NR_026731 . . . 1_ | 2261 | 104.2995 |
| | merge 2261-2280_as | | |
| LINC01551 | NR_026732.1_NR_026731 . . . 1_ | 2262 | 125.7552 |
| | merge 2262-2281_as | | |
| LINC01551 | NR_026732.1_NR_026731 . . . 1_ | 2319 | 112.9311 |
| | merge_2319-2338_as | | |
| LINC01551 | NR_026732.1_NR_026731 . . . 1_ | 2327 | 122.0279 |
| | merge_2327-2346_as | | |
| LINC01551 | NR_026732.1_NR_026731 . . . 1_ | 2334 | 116.5958 |
| | merge_2334-2353_as | | |
| LINC01551 | NR_026732.1_NR_026731 . . . 1_ | 2335 | 145.574 |
| | merge_2335-2354_as | | |
| LINC01551 | NR_026732.1_NR_026731 . . . 1_ | 2336 | 128.9508 |
| | merge_2336-2355_as | | |
| LINC01551 | NR_026732.1_NR_026731 . . . 1_ | 2337 | 123.1395 |
| | merge_2337-2356_as | | |
| LINC01551 | NR_026732.1_NR_026731 . . . 1_ | 2338 | 144.2022 |
| | merge_2338-2357_as | | |
| LINC01551 | NR_026732.1_NR_026731 . . . 1_ | 2339 | 126.7695 |
| | merge_2339-2358_as | | |
| LINC01551 | NR_026732.1_NR_026731 . . . 1_ | 2340 | 133.0967 |
| | merge_2340-2359_as | | |
| LINC01551 | NR_026732.1_NR_026731 . . . 1_ | 2341 | 137.2337 |
| | merge_2341-2360_as | | |
| LINC01551 | NR_026732.1_NR_026731 . . . 1_ | 2342 | 116.1773 |
| | merge_2342-2361_as | | |
| LINC01551 | NR_026732.1_NR_026731 . . . 1_ | 2372 | 149.8658 |
| | merge_2372-2391_as | | |
| LINC01551 | NR_026732.1_NR_026731 . . . 1_ | 2428 | 116.4803 |
| | merge_2428-2447_as | | |
| LINC01551 | NR_026732.1_NR_026731 . . . 1_ | 2429 | 113.4778 |
| | merge_2429-2448_as | | |
| LINC01551 | NR_026732.1_NR_026731 . . . 1_ | 2431 | 124.9652 |
| | merge_2431-2450_as | | |
| LINC01551 | NR_026732.1_NR_026731 . . . 1_ | 2675 | 107.9292 |
| | merge_2675-2694_as | | |
| LINC01551 | NR_026732.1_NR_026731 . . . 1_ | 2736 | 149.4117 |
| | merge_2736-2755_as | | |
| LINC01551 | NR_026732.1_NR_026731 . . . 1_ | 3554 | 208.3008 |
| | merge_3554-3573_as | | |
| LINC01551 | NR_026732.1_NR_026731 . . . 1_ | 3673 | 132.79 |
| | merge_3673-3692_as | | |
| LINC01551 | NR_026732.1_NR_026731 . . . 1_ | 3710 | 109.4747 |
| | merge_3710-3729_as | | |
| LINC01551 | NR_026732.1_NR_026731 . . . 1_ | 3711 | 112.2256 |
| | merge_3711-3730_as | | |
| LINC02282 | NR_135255.1_89-108_as | 89 | 119.5063 |
| LINC02282 | NR_135255.1_90-109_as | 90 | 105.3562 |
| LINC02282 | NR_135255.1_192-211_as | 192 | 148.5745 |
| LINC02282 | NR_135255.1_193-212_as | 193 | 130.9477 |
| LINC02282 | NR_135255.1_194-213_as | 194 | 112.1581 |
| LINC02282 | NR_135255.1_204-223_as | 204 | 157.1731 |
| LINC02282 | NR_135255.1_230-249_as | 230 | 133.4666 |
| LINC02282 | NR_135255.1_232-251_as | 232 | 113.5322 |
| LINC02282 | NR_135255.1_233-252_as | 233 | 127.2036 |
| LINC02282 | NR_135255.1_234-253_as | 234 | 146.0912 |
| LINC02282 | NR_135255.1_235-254_as | 235 | 134.7058 |
| LINC02282 | NR_135255.1_236-255_as | 236 | 142.5579 |
| LINC02282 | NR_135255.1_237-256_as | 237 | 131.8185 |
| LINC02282 | NR_135255.1_243-262_as | 243 | 135.5674 |
| LINC02282 | NR_135255.1_456-475_as | 456 | 101.1669 |
| LINC02282 | NR_135255.1_462-481_as | 462 | 182.4739 |
| LINC02282 | NR_135255.1_586-605_as | 586 | 111.6677 |
| LINC02282 | NR_135255.1_669-688_as | 669 | 108.6124 |
| LINC02282 | NR_135255.1_670-689_as | 670 | 109.8295 |
| LINC02282 | NR_135255.1_672-691_as | 672 | 109.6542 |
| LINC02282 | NR_135255.1_674-693_as | 674 | 122.3342 |
| LINC02282 | NR_135255.1_675-694_as | 675 | 100.8784 |
| LINC02282 | NR_135255.1_680-699_as | 680 | 105.2365 |
| LINC02282 | NR_135255.1_763-782_as | 763 | 145.577 |
| LINC02282 | NR_135255.1_764-783_as | 764 | 133.2802 |
| LINC02282 | NR_135255.1_766-785_as | 766 | 109.5801 |
| LINC02282 | NR_135255.1_768-787_as | 768 | 102.8706 |
| LINC02282 | NR_135255.1_769-788_as | 769 | 111.9311 |
| LINC02282 | NR_135255.1_770-789_as | 770 | 102.1801 |
| LINC02282 | NR_135255.1_771-790_as | 771 | 102.3618 |
| LINC02282 | NR_135255.1_772-791_as | 772 | 119.2151 |
| LINC02282 | NR_135255.1_833-852_as | 833 | 159.2383 |
| LINC02282 | NR_135255.1_834-853_as | 834 | 153.6097 |
| LINC02282 | NR_135255.1_835-854_as | 835 | 220.5611 |
| LINC02282 | NR_135255.1_836-855_as | 836 | 176.0932 |
| LINC02282 | NR_135255.1_837-856_as | 837 | 184.2671 |
| LINC02282 | NR_135255.1_838-857_as | 838 | 155.0812 |
| LINC02282 | NR_135255.1_839-858_as | 839 | 147.9743 |
| LINC02282 | NR_135255.1_840-859_as | 840 | 163.6664 |
| LINC02282 | NR_135255.1_841-860_as | 841 | 122.8392 |
| LINC02282 | NR_135255.1_843-862_as | 843 | 115.8296 |
| LINC02282 | NR_135255.1_844-863_as | 844 | 106.7405 |
| LINC02282 | NR_135255.1_845-864_as | 845 | 113.1295 |
| LINC02282 | NR_135255.1_862-881_as | 862 | 153.6961 |
| LINC02282 | NR_135255.1_863-882_as | 863 | 103.0357 |
| LINC02282 | NR_135255.1_867-886_as | 867 | 136.8735 |
| LINC02282 | NR_135255.1_869-888_as | 869 | 179.7552 |
| LINC02282 | NR_135255.1_870-889_as | 870 | 117.8833 |
| LINC02282 | NR_135255.1_876-895_as | 876 | 112.4724 |
| LINC02282 | NR_135255.1_979-998_as | 979 | 139.6044 |
| LINC02282 | NR_135255.1_981-1000_as | 981 | 140.7416 |
| LINC02282 | NR_135255.1_982-1001_as | 982 | 215.5858 |
| LINC02282 | NR_135255.1_983-1002_as | 983 | 176.4842 |
| LINC02282 | NR_135255.1_984-1003_as | 984 | 125.5474 |
| LINC02282 | NR_135255.1_1226-1245_as | 1226 | 113.2286 |
| LINC02282 | NR_135255.1_1229-1248_as | 1229 | 129.359 |
| LINC02282 | NR_135255.1_1327-1346_as | 1327 | 134.7263 |
| LINC02282 | NR_135255.1_1328-1347_as | 1328 | 163.2255 |
| LINC02282 | NR_135255.1_1329-1348_as | 1329 | 191.2834 |
| LINC02282 | NR_135255.1_1330-1349_as | 1330 | 187.9057 |
| LINC02282 | NR_135255.1_1536-1555_as | 1536 | 129.409 |
| LINC02282 | NR_135255.1_1537-1556_as | 1537 | 126.2619 |

TABLE 4
Antisense oligonucleotides (ASOs) increasing FOXG1 expression

| Target | Oligo ID | Position |
|---|---|---|
| FOXG1-AS1 | NR_125758.1_206-225_as | 206 |
| FOXG1-AS1 | NR_125758.1_208-227_as | 208 |
| FOXG1-AS1 | NR_125758.1_209-228_as | 209 |
| FOXG1-AS1 | NR_125758.1_297-316_as | 297 |
| FOXG1-AS1 | NR_125758.1_416-435_as | 416 |
| LINC01551 | NR_026732.1_NR_026731.1_ | 964 |
| | merge_964-983_as | |
| LINC01551 | NR_026732.1_NR_026731.1_ | 1046 |
| | merge_1046-1065_as | |
| LINCO1551 | NR_026732.1_NR_026731.1_ | 1047 |
| | merge_1047-1066_as | |
| LINC01551 | NR_026732.1_NR_026731.1_ | 1049 |
| | merge_1049-1068_as | |
| LINC01551 | NR_026732.1_NR_026731.1_ | 1050 |
| | merge_1050-1069_as | |
| LINCO1551 | NR_026732.1_NR_026731.1_ | 1051 |
| | merge_1051-1070_as | |
| LINC01551 | NR_026732.1_NR_026731.1_ | 1052 |
| | merge_1052-1071_as | |
| LINC01551 | NR_026732.1_NR_026731.1_ | 1053 |
| | merge_1053-1072_as | |
| LINC01551 | NR_026732.1_NR_026731.1_ | 1054 |
| | merge_1054-1073_as | |
| LINC01551 | NR_026732.1_NR_026731.1_ | 1550 |
| | merge_1550-1569_as | |
| LINC01551 | NR_026732.1_NR_026731.1_ | 2247 |
| | merge_2247-2266_as | |
| LINC01551 | NR_026732.1_NR_026731.1_ | 2248 |
| | merge_2248-2267_as | |
| LINC01551 | NR_026732.1_NR_026731.1_ | 2253 |
| | merge_2253-2272_as | |
| LINCO1551 | NR_026732.1_NR_026731.1_ | 3554 |
| | merge_3554-3573_as | |
| LINC02282 | NR_135255.1_192-211_as | 192 |
| LINCO2282 | NR_135255.1_204-223_as | 204 |
| LINCO2282 | NR_135255.1_462-481_as | 462 |
| LINCO2282 | NR_135255.1_833-852_as | 833 |
| LINCO2282 | NR_135255.1_834-853_as | 834 |
| LINCO2282 | NR_135255.1_835-854_as | 835 |
| LINCO2282 | NR_135255.1_836-855_as | 836 |
| LINC02282 | NR_135255.1_837-856_as | 837 |
| LINCO2282 | NR_135255.1_838-857_as | 838 |
| LINCO2282 | NR_135255.1_839-858_as | 839 |
| LINCO2282 | NR_135255.1_840-859_as | 840 |
| LINCO2282 | NR_135255.1_862-881_as | 862 |
| LINC02282 | NR_135255.1_869-888_as | 869 |
| LINC02282 | NR_135255.1_982-1001_as | 982 |
| LINCO2282 | NR_135255.1_983-1002_as | 983 |
| LINCO2282 | NR_135255.1_1328-1347_as | 1328 |
| LINCO2282 | NR_135255.1_1329-1348_as | 1329 |
| LINC02282 | NR_135255.1_1330-1349_as | 1330 |

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the present disclosure. It is intended that the following claims define the scope of the present disclosure and that methods and structures within the scope of these claims and their equivalents be covered thereby.

SEQUENCE LISTING

| SEQ ID NO: | Sequence |
|---|---|
| 1 | GGTATGTTTCGTGCCCATGT |
| 2 | GTGGTATGTTTCGTGCCCAT |
| 3 | TGTGGTATGTTTCGTGCCCA |
| 4 | ATGTGGTATGTTTCGTGCCC |
| 5 | AATGTGGTATGTTTCGTGCC |
| 6 | AAATGTGGTATGTTTCGTGC |
| 7 | AAAATGTGGTATGTTTCGTG |
| 8 | TAAAATGTGGTATGTTTCGT |
| 9 | GTAAAATGTGGTATGTTTCG |
| 10 | CGTAAAATGTGGTATGTTTC |
| 11 | CCGTAAAATGTGGTATGTTT |
| 12 | TCCGTAAAATGTGGTATGTT |
| 13 | GGCTACATCCTCCGTAAAAT |
| 14 | TCATTTATGCTTCTCCACCT |
| 15 | TTCATTTATGCTTCTCCACC |
| 16 | TTTCATTTATGCTTCTCCAC |
| 17 | CTTTCATTTATGCTTCTCCA |
| 18 | CCTTTCATTTATGCTTCTCC |
| 19 | GCCTTTCATTTATGCTTCTC |
| 20 | TGCCTTTCATTTATGCTTCT |
| 21 | GTGCCTTTCATTTATGCTTC |
| 22 | GGTGCCTTTCATTTATGCTT |
| 23 | AGGTGCCTTTCATTTATGCT |
| 24 | AAGGTGCCTTTCATTTATGC |
| 25 | AATTCTCTGTGCATCTTCTA |
| 26 | AAATTCTCTGTGCATCTTCT |
| 27 | GAAATTCTCTGTGCATCTTC |
| 28 | AGAAATTCTCTGTGCATCTT |
| 29 | CACAAGGTCAGAAATTCTCT |
| 30 | TCACAAGGTCAGAAATTCTC |
| 31 | GTCACAAGGTCAGAAATTCT |
| 32 | AACGTCACAAGGTCAGAAAT |
| 33 | TGGATGCCTCTGTATGGGAT |
| 34 | CTGGATGCCTCTGTATGGGA |
| 35 | ACCTGGATGCCTCTGTATGG |
| 36 | TACCTGGATGCCTCTGTATG |
| 37 | ATACCTGGATGCCTCTGTAT |
| 38 | AATACCTGGATGCCTCTGTA |
| 39 | AAATACCTGGATGCCTCTGT |
| 40 | GAAATACCTGGATGCCTCTG |
| 41 | GGAAATACCTGGATGCCTCT |
| 42 | ATTATAGACGAGTTGGCTCC |
| 43 | GCTGTTAGGAAGATATTATA |
| 44 | TGCTGTTAGGAAGATATTAT |
| 45 | CTGCTGTTAGGAAGATATTA |
| 46 | TCTGCTGTTAGGAAGATATT |
| 47 | TTCTGCTGTTAGGAAGATAT |
| 48 | GTTCTGCTGTTAGGAAGATA |
| 49 | GGTTCTGCTGTTAGGAAGAT |
| 50 | AGGTTCTGCTGTTAGGAAGA |
| 51 | CAGGTTCTGCTGTTAGGAAG |
| 52 | CCAGGTTCTGCTGTTAGGAA |
| 53 | CCCAGGTTCTGCTGTTAGGA |
| 54 | ACCCAGGTTCTGCTGTTAGG |
| 55 | GAGACCCAGGTTCTGCTGTT |
| 56 | TGAGACCCAGGTTCTGCTGT |
| 57 | CCGTACCTGTAGTTCCAGCT |
| 58 | TCCCGTACCTGTAGTTCCAG |
| 59 | TTCCCGTACCTGTAGTTCCA |
| 60 | TTTCCCGTACCTGTAGTTCC |
| 61 | TTTTCCCGTACCTGTAGTTC |
| 62 | GTTTTCCCGTACCTGTAGTT |
| 63 | AGTTTTCCCGTACCTGTAGT |
| 64 | CCGAAATTATTTTGTTAAAC |
| 65 | GCCGAAATTATTTTGTTAAA |
| 66 | AGCCGAAATTATTTTGTTAA |
| 67 | TAGCCGAAATTATTTTGTTA |
| 68 | ATAGCCGAAATTATTTTGTT |
| 69 | GATAGCCGAAATTATTTTGT |
| 70 | TTGATAGCCGAAATTATTTT |
| 71 | TTTGATAGCCGAAATTATTT |
| 72 | CTTTGATAGCCGAAATTATT |
| 73 | TCTTTGATAGCCGAAATTAT |
| 74 | GATCTTTGATAGCCGAAATT |
| 75 | TGATCTTTGATAGCCGAAAT |
| 76 | TTGATCTTTGATAGCCGAAA |
| 77 | CTTGATCTTTGATAGCCGAA |
| 78 | ACTTGATCTTTGATAGCCGA |
| 79 | CACTTGATCTTTGATAGCCG |
| 80 | CCACTTGATCTTTGATAGCC |
| 81 | TATCCCACTTGATCTTTGAT |
| 82 | TTATCCCACTTGATCTTTGA |
| 83 | ATTTATCCCACTTGATCTTT |
| 84 | AATTTATCCCACTTGATCTT |
| 85 | CCTCTATGGTATGCAAGGAG |
| 86 | ACCTCGACCTCTCCTCTATG |
| 87 | GACCTCGACCTCTCCTCTAT |
| 88 | GCTAGCAGACTCACACCACA |
| 89 | TCACGGCTAGCAGACTCACA |
| 90 | TGTCTCTCACGGCTAGCAGA |
| 91 | TCTGTCTCTCACGGCTAGCA |
| 92 | ATCTGTCTCTCACGGCTAGC |
| 93 | CCCTTTGTAATGCATCTGTC |
| 94 | TCCCTTTGTAATGCATCTGT |
| 95 | ATCCCTTTGTAATGCATCTG |
| 96 | CATCCCTTTGTAATGCATCT |
| 97 | CCATCCCTTTGTAATGCATC |
| 98 | TCCATCCCTTTGTAATGCAT |
| 99 | ATCCATCCCTTTGTAATGCA |
| 100 | AATCCATCCCTTTGTAATGC |
| 101 | AAATCCATCCCTTTGTAATG |
| 102 | TAAATCCATCCCTTTGTAAT |
| 103 | CTAAATCCATCCCTTTGTAA |
| 104 | ACTAAATCCATCCCTTTGTA |
| 105 | CACTAAATCCATCCCTTTGT |
| 106 | GCACTAAATCCATCCCTTTG |
| 107 | TGCACTAAATCCATCCCTTT |
| 108 | GTGCACTAAATCCATCCCTT |
| 109 | AGTGCACTAAATCCATCCCT |
| 110 | GTTTTGTTTCATTGTTCACT |
| 111 | AGTTTTGTTTCATTGTTCAC |
| 112 | AAGTTTTGTTTCATTGTTCA |
| 113 | CTTGGGAAGAAGTTTTGTTT |
| 114 | GCTTGGGAAGAAGTTTTGTT |
| 115 | ATCTCTTCAAACTATGGCAC |
| 116 | CATCTCTTCAAACTATGGCA |
| 117 | TGCCATCTCTTCAAACTATG |
| 118 | ATGCCATCTCTTCAAACTAT |
| 119 | GATGCCATCTCTTCAAACTA |
| 120 | TTGTATAAACTGTTGTTGCA |
| 121 | GAAGCTGAAGTGGTGTTGGG |
| 122 | AGAAGCTGAAGTGGTGTTGG |
| 123 | CTTTTCCTCGGCATCCTTCG |
| 124 | CCTTTTCCTCGGCATCCTTC |
| 125 | TCCTTTTCCTCGGCATCCTT |
| 126 | ATCCTTTTCCTCGGCATCCT |
| 127 | TATCCTTTTCCTCGGCATCC |
| 128 | ATATCCTTTTCCTCGGCATC |
| 129 | GATATCCTTTTCCTCGGCAT |
| 130 | TGATATCCTTTTCCTCGGCA |
| 131 | CCGATGCTCTGGAATCTCAA |
| 132 | TCCGATGCTCTGGAATCTCA |
| 133 | CATCCGATGCTCTGGAATCT |
| 134 | TCATCCGATGCTCTGGAATC |
| 135 | TTCATCCGATGCTCTGGAAT |
| 136 | ACTACCCCTATGCACGTGAG |
| 137 | GTTCTTCCCCAAATGCCTTT |
| 138 | TGTTCTTCCCCAAATGCCTT |
| 139 | TTGTTCTTCCCCAAATGCCT |
| 140 | GTTGTTCTTCCCCAAATGCC |
| 141 | CGTTGTTCTTCCCCAAATGC |
| 142 | CTTTCTCTGGAGACACATCA |
| 143 | ACTTTCTCTGGAGACACATC |
| 144 | GTTGTTTGTTTGTTTGTTTT |
| 145 | TGTTGTTTGTTTGTTTGTTT |
| 146 | TTGTTGTTTGTTTGTTTGTT |
| 147 | GTTGTTGTTTGTTTGTTTGT |
| 148 | TGTTGTTGTTTGTTTGTTTG |
| 149 | TTGTTGTTGTTTGTTTGTTT |
| 150 | ATTGTTGTTGTTTGTTTGTT |
| 151 | TATTGTTGTTGTTTGTTTGT |
| 152 | TTATTGTTGTTGTTTGTTTG |
| 153 | GTTTATTGTTGTTGTTTGTT |
| 154 | TGTTTATTGTTGTTGTTTGT |
| 155 | TTGTTTATTGTTGTTGTTTG |
| 156 | GTTGTTTATTGTTGTTGTTT |
| 157 | AGTTGTTTATTGTTGTTGTT |
| 158 | AAGTTGTTTATTGTTGTTGT |
| 159 | AGTGGAATGAGTCAGCCCGA |
| 160 | AAGTGGAATGAGTCAGCCCG |
| 161 | AAAGTGGAATGAGTCAGCCC |
| 162 | CCTGCTGGATAGGAATTAAT |
| 163 | GCCTGCTGGATAGGAATTAA |
| 164 | TTAAAGCCTGCTGGATAGGA |
| 165 | TGTTAAAGCCTGCTGGATAG |
| 166 | TTGTTAAAGCCTGCTGGATA |
| 167 | TTTGTTAAAGCCTGCTGGAT |
| 168 | TTTTGTTAAAGCCTGCTGGA |
| 169 | TTTTTGTTAAAGCCTGCTGG |
| 170 | GTTTTTGTTAAAGCCTGCTG |
| 171 | AGTTTTTGTTAAAGCCTGCT |
| 172 | TAGTTTTTGTTAAAGCCTGC |
| 173 | TCTTTAGTAGCTTTCATGGC |
| 174 | CTGGCTTTTCTTTAGTAGCT |
| 175 | GCTGTTTCTGGCTTTTCTTT |
| 176 | CGCTGTTTCTGGCTTTTCTT |
| 177 | ACGCTGTTTCTGGCTTTTCT |
| 178 | TACGCTGTTTCTGGCTTTTC |
| 179 | TTACGCTGTTTCTGGCTTTT |
| 180 | CTTACGCTGTTTCTGGCTTT |
| 181 | TCTTACGCTGTTTCTGGCTT |
| 182 | TTCTTACGCTGTTTCTGGCT |
| 183 | ATTCTTACGCTGTTTCTGGC |
| 184 | CCTCGTCTCTGAATCATATT |
| 185 | CACAATAGTAGTGGCCTTGT |
| 186 | TCACAATAGTAGTGGCCTTG |
| 187 | TTCACAATAGTAGTGGCCTT |
| 188 | ATTCACAATAGTAGTGGCCT |
| 189 | CCATGTTGACTTAGTTGGTC |
| 190 | ACCATGTTGACTTAGTTGGT |
| 191 | GCTACCATGTCTGACTAATT |
| 192 | TGCTACCATGTCTGACTAAT |
| 193 | GTGCTACCATGTCTGACTAA |
| 194 | TGTGCTACCATGTCTGACTA |
| 195 | ATGTGCTACCATGTCTGACT |
| 196 | CATGTGCTACCATGTCTGAC |
| 197 | TGGGTGATATTTGGTTCCAA |
| 198 | CTGAGGAAATTGATGGTATA |
| 199 | ACTGAGGAAATTGATGGTAT |
| 200 | GACCGTACGAGGGAATTTTA |
| 201 | TGACCGTACGAGGGAATTTT |
| 202 | TTGACCGTACGAGGGAATTT |
| 203 | TTTGACCGTACGAGGGAATT |
| 204 | TTTTGACCGTACGAGGGAAT |
| 205 | GCCTTCTGTACTGTGATGGG |
| 206 | AGCCTTCTGTACTGTGATGG |
| 207 | AAGCCTTCTGTACTGTGATG |
| 208 | ATGTGTGGGATGTAGGTAGG |
| 209 | AATGTGTGGGATGTAGGTAG |
| 210 | AAATGTGTGGGATGTAGGTA |
| 211 | GGGACTCCTGAAATGTGTGG |
| 212 | CGTTCTGTGTTTTGTAGAAT |
| 213 | GTCGTTCTGTGTTTTGTAGA |
| 214 | GGTCGTTCTGTGTTTTGTAG |
| 215 | TGGTCGTTCTGTGTTTTGTA |
| 216 | ATGGTCGTTCTGTGTTTTGT |
| 217 | TATGGTCGTTCTGTGTTTTG |
| 218 | ATATGGTCGTTCTGTGTTTT |
| 219 | TGGCTCATATGGTCGTTCTG |
| 220 | GTGGCTCATATGGTCGTTCT |
| 221 | AGTGGCTCATATGGTCGTTC |
| 222 | AAGTGGCTCATATGGTCGTT |
| 223 | CTCAGTGACAGCTAGGTGGA |
| 224 | TCTCAGTGACAGCTAGGTGG |
| 225 | ATTCTCAGTGACAGCTAGGT |
| 226 | CCGAATTCTCAGTGACAGCT |
| 227 | CAATGCAGAGTTTCTATTAC |
| 228 | CCCATTCCCAGGATGTTAGA |
| 229 | TCCCATTCCCAGGATGTTAG |
| 230 | TTCCCATTCCCAGGATGTTA |
| 231 | CTTCCCATTCCCAGGATGTT |
| 232 | ACTTCCCATTCCCAGGATGT |
| 233 | TACTTCCCATTCCCAGGATG |
| 234 | TTACTTCCCATTCCCAGGAT |
| 235 | GTTACTTCCCATTCCCAGGA |
| 236 | TGTTACTTCCCATTCCCAGG |
| 237 | GTGTTACTTCCCATTCCCAG |
| 238 | AGTGTTACTTCCCATTCCCA |
| 239 | CAGTGTTACTTCCCATTCCC |
| 240 | CCACCGATCCCAGTGTTACT |
| 241 | TTTCCATTCCTCTCTTCCAT |
| 242 | CTTTCCATTCCTCTCTTCCA |
| 243 | GCCTTTCCATTCCTCTCTTC |
| 244 | TGCCTTTCCATTCCTCTCTT |
| 245 | TTGCCTTTCCATTCCTCTCT |
| 246 | TTTGCCTTTCCATTCCTCTC |
| 247 | TTTTGCCTTTCCATTCCTCT |
| 248 | CTTTTGCCTTTCCATTCCTC |
| 249 | TCTTTTGCCTTTCCATTCCT |
| 250 | TTCTTTTGCCTTTCCATTCC |
| 251 | TGCTGATGGTGGGACTTTTT |
| 252 | TTGCTGATGGTGGGACTTTT |
| 253 | TTTGCTGATGGTGGGACTTT |
| 254 | TTTTGCTGATGGTGGGACTT |
| 255 | CTTTTGCTGATGGTGGGACT |
| 256 | TCTTTTGCTGATGGTGGGAC |
| 257 | TTCTTTTGCTGATGGTGGGA |
| 258 | CTTCTTTTGCTGATGGTGGG |
| 259 | ACTTCTTTTGCTGATGGTGG |
| 260 | GACTTCTTTTGCTGATGGTG |
| 261 | AGACTTCTTTTGCTGATGGT |
| 262 | GAGACTTCTTTTGCTGATGG |
| 263 | AGAGACTTCTTTTGCTGATG |
| 264 | GCTGCTATTTTAGAGGAAGA |
| 265 | GGCTGCTATTTTAGAGGAAG |
| 266 | CTTTGGCTGCTATTTTAGAG |
| 267 | CTCTTTGGCTGCTATTTTAG |
| 268 | TCTCTTTGGCTGCTATTTTA |
| 269 | ATTTTCTCTCTTTGGCTGCT |
| 270 | GTTCAGAAATTGGGATTAAT |
| 271 | TGTTCAGAAATTGGGATTAA |
| 272 | GCTGTTCAGAAATTGGGATT |
| 273 | TGCTGTTCAGAAATTGGGAT |
| 274 | ATGCTGTTCAGAAATTGGGA |
| 275 | AATGCTGTTCAGAAATTGGG |
| 276 | GCTAAGTAAAATGCTGTTCA |
| 277 | TGCTAAGTAAAATGCTGTTC |
| 278 | TTTCCAACAGGCTCTCGTTT |
| 279 | CTTTCCAACAGGCTCTCGTT |
| 280 | CCTTTCCAACAGGCTCTCGT |
| 281 | TCCTTTCCAACAGGCTCTCG |
| 282 | GGTAGAATGGGAAAGGTTTT |
| 283 | GGGTAGAATGGGAAAGGTTT |
| 284 | TGGGTAGAATGGGAAAGGTT |
| 285 | CTGGGTAGAATGGGAAAGGT |
| 286 | GCACAAGTGGCAAAGCAAAA |
| 287 | TGCACAAGTGGCAAAGCAAA |
| 288 | AGATCTGTTGCACAAGTGGC |

1. A gapmer antisense oligonucleotide comprising a sequence that hybridizes to a target nucleic acid sequence of a long non-coding RNA (lncRNA), wherein the gapmer antisense oligonucleotide comprises a structure having the formula: 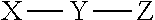
wherein:
X is a 5′ wing region,
Y is a gap region, and
Z is a 3′ wing region; and
wherein X, Y, and/or Z comprise one or more modifications.

2. The gapmer antisense oligonucleotide of claim 1, wherein the lncRNA regulates expression of FOXG1.

3. The gapmer antisense oligonucleotide of claim 2, wherein the lncRNA reduces expression of FOXG1 messenger RNA.

4. The gapmer antisense oligonucleotide of claim 2, wherein the lncRNA reduces transcription of FOXG1 messenger RNA molecule.

5. The gapmer antisense oligonucleotide of claim 2, wherein the lncRNA reduces expression of FOXG1 protein.

6. The gapmer antisense oligonucleotide of claim 2, wherein the lncRNA reduces translation of a FOXG1 protein molecule.

7. The gapmer antisense oligonucleotide of any one of claims 1 to 6, wherein X comprises one or more nucleoside modifications.

8. The gapmer antisense oligonucleotide of any one of claims 1 to 7, wherein Z comprises one or more nucleoside modifications.

9. The gapmer antisense oligonucleotide of any one of claims 1 to 8, wherein X and Z each comprise one or more nucleoside modifications.

10. The gapmer antisense oligonucleotide of any one of claims 1 to 9, wherein the one or more nucleoside modifications comprises a LNA (2′-4′ biradicle bridged) nucleoside, 2′ substituted nucleoside (e.g., 2′-O-alkyl-RNA, 2′-O-methyl-RNA, 2′-alkoxy-RNA, 2′-O-methoxyethyl-oligos (MOE), 2′-amino-DNA, 2′-Fluoro-RNA, and 2′-F-ANA nucleosides), nucleobase modifications. Or a combination thereof.

11. The gapmer antisense oligonucleotide of any one of claims 1 to 10, wherein X comprises one or more inter-nucleoside linker modifications.

12. The gapmer antisense oligonucleotide of any one of claims 1 to 11, wherein Y comprises one or more inter-nucleoside linker modifications.

13. The gapmer antisense oligonucleotide of any one of claims 1 to 12, wherein Z comprises one or more inter-nucleoside linker modifications.

14. The gapmer antisense oligonucleotide of any one of claims 1 to 13, wherein X and Z each comprise one or more inter-nucleoside linker modifications.

15. The gapmer antisense oligonucleotide of any one of claims 1 to 13, wherein X, Y, and Z each comprise one or more inter-nucleoside linker modifications.

16. The gapmer antisense oligonucleotide of any one of claims 1 to 15, wherein the sequence is complementary to the target nucleic acid sequence of a long non-coding RNA (lncRNA).

17. The gapmer antisense oligonucleotide of any one of claims 1 to 16, wherein the long non-coding RNA (lncRNA) is located within 1 kilobases (kb), 2 kb, 5 kb, 8 kb, or 10 kb of a gene encoding FOXG1.

18. The gapmer antisense oligonucleotide of any one of claims 1 to 17, wherein the long non-coding RNA (lncRNA) is FOXG1-AS1, long intergenic non-protein coding RNA 1551, long intergenic non-protein coding RNA 2282 (LINC02282), or a combination thereof.

19. The gapmer antisense oligonucleotide of any one of claims 1 to 18, wherein the sequence comprises a nucleobase sequence as set forth in any one of Table 3.

20. The gapmer antisense oligonucleotide of any one of claims 1 to 18, wherein the gapmer antisense oligonucleotide comprises a nucleobase sequence as set forth in any one of Table 4.

21. The gapmer antisense oligonucleotide of any one of claims 1 to 18, wherein the gapmer antisense oligonucleotide hybridizes to the target nucleic acid sequence comprising or adjacent to any one or more of the positions provided in Table 3.

22. The gapmer antisense oligonucleotide of claim 21, wherein adjacent to any one or more of the positions provided in Table 3 comprises base positions within 20, 40, 50, 75, 100, or 150 base positions 5′ and/or 3′.

23. The gapmer antisense oligonucleotide of any one of claims 1 to 22, wherein the gapmer antisense oligonucleotide hybridizes to the target nucleic acid sequence comprising or adjacent to any one or more of the positions provided in Table 4,

24. The gapmer antisense oligonucleotide of claim 23, wherein adjacent to any one or more of the positions provided in Table 4 comprises base positions within 20, 40, 50, 75, 100, or 150 base positions 5′ and/or 3′.

25. The gapmer antisense oligonucleotide of any one of claims 1 to 24, wherein hybridization of the sequence of the gapmer antisense oligonucleotide to the target nucleic acid sequence increases degradation of the lncRNA.

26. The gapmer antisense oligonucleotide of any one of claims 1 to 24, wherein hybridization of the sequence of the gapmer antisense oligonucleotide to the target nucleic acid sequence increases expression of FOXG1.

27. The gapmer antisense oligonucleotide of claim 26, wherein expression of FOXG1 is mRNA expression.

28. The gapmer antisense oligonucleotide of claim 26, wherein expression of FOXG1 is protein expression.

29. A composition comprising one or more of the gapmer antisense oligonucleotides of any one of claims 1 to 28.

30. A pharmaceutical composition comprising the gapmer antisense oligonucleotide of any one of claims 1 to 29 and a pharmaceutically acceptable carrier or diluent.

31. A method of modulating expression of FOXG1 in a cell, comprising contacting the cell with the gapmer antisense oligonucleotide of any one of claims 1 to 29.

32. The method of claim 31, wherein the cell is a located in a brain of an individual.

33. The method of claim 32, wherein the individual is a human.

34. The method of claim 32, wherein the individual comprises reduced FOXG1 expression or a FOXG1 deficiency.

35. The method of claim 32, wherein the individual has a FOXG1 disease or disorder.

36. The method of claim 35, wherein the FOXG1 disease or disorder is FOXG1 syndrome.

37. The method of any one of claims 31 to 36, wherein modulating expression comprises increasing expression of a FOXG1 protein in the cell.

38. The method of any one of claims 31 to 38, wherein modulating expression comprises increasing translation of a FOXG1 protein in the cell.

39. A method of treating or ameliorating a FOXG1 disease or disorder in an individual having, or at risk of having, the FOXG1 disease or disorder, comprising administering to the individual the gapmer antisense oligonucleotide of any one of claims 1 to 29.

40. The method of claim 39, wherein the individual is a human.

41. The method of claim 40, wherein the human is an unborn human.

42. The method of any one of claims 39 to 41, wherein the individual comprises a mutated FOXG1 gene, a reduced expression of FOXG1, a deficiency of FOXG1, or a combination thereof.

43. The method of any one of claims 39 to 42, wherein the FOXG1 disease or disorder is FOXG1 syndrome.
